(19) United States
(12) Patent Application Publication
(10) Pub. No.: US 2025/0339374 A1
(43) Pub. Date: Nov. 6, 2025

(54) BONDED MICROPARTICULATES PREPARATION AND APPLICATIONS THEREOF

(71) Applicant: Venkor Corporation, Centreville, VA (US)

(72) Inventor: Subraman Rao Cherukuri, Fairfax, VA (US)

(21) Appl. No.: 18/857,153

(22) PCT Filed: May 31, 2022
(86) PCT No.: PCT/US2022/031669

§ 371 Date: Oct. 15, 2024

(51) Int. Cl. A61K 9/16; A61K 31/047; A61K 31/09; A61K 31/135; A61K 31/155; A61K 31/19; A61K 31/191; A61K 31/192; A61K 31/4422; A61K 31/455; A61K 31/519; A61K 31/702; A61K 45/06

(52) U.S. Cl. CPC A61K 9/1641; A61K 9/1617; A61K 31/047; A61K 31/09; A61K 31/135; A61K 31/155; A61K 31/19; A61K 31/191; A61K 31/192; A61K 31/4422; A61K 31/455; A61K 31/519; A61K 31/702; A61K 45/06

(57) ABSTRACT

Bonded microparticulate (BMPX) compositions comprising a combination of a solid particulate material and a bonding agent and methods for the manufacture and use thereof are disclosed and described. Additionally, formulations and dosage forms including or otherwise prepared from such BMPX compositions are also disclosed and described.

### FIELD OF THE DISCLOSURE

The present disclosure relates to bonded microparticulates, composition and processes thereof, and their application to wide variety of solid particulate material with varying density, solubility and form and utility in medical and consumer products for human and animal use, such as pharmaceuticals, biologics, dietary supplements and food products.

### BACKGROUND OF THE DISCLOSURE

Granulation techniques are widely used in various industries, for example, in the production of pharmaceuticals, dietary supplements, and food products. Granulation processes typically transform fine powders into free-flowing, dust-free granules that can be compressed. Granulation is important to facilitate handling and product formation in many industrial processes.

Generally, granulation commences after initial dry mixing of the necessary powder ingredients along with any active ingredients, so that a uniform distribution of each ingredient throughout the mixture is achieved. Granule properties play an important role in the overall compression and compactness of a final product (e.g. a pharmaceutical dosage form), and the dissolution and/or disintegration properties any consolidated mass formed from the granules (e.g. an oral dosage tablet). The flow and filling of a compression die, ejection of dosage form without sticking, picking and consistent weight, appearance, friability, hardness, uniform distribution of active agent, dissolution or disintegration properties and many other Pharmacopeia standard tests of a tablet or consolidate can be of critical importance in a finished product (e.g. a pharmaceutical product).

Generally, there are two dominant types of granulation methods: “wet granulation” and “dry granulation”. The specific method selected largely depends on the properties of the ingredients (e.g., active ingredients, excipients, flavors, and the like) to be formulated into granules. The specific of process selected requires thorough knowledge of physicochemical properties of the drug, excipients, required flow and release properties. Specific exemplary granulation technologies include roller compaction, extrusion/spheronization, spray drying, supercritical fluid, low/High shear mixing, fluid bed granulation, reverse wet granulation, hotmelt granulation, freeze granulation, melt granulation and foam granulation.

Wet granulation methods typically include the steps of: mixing or blending ingredients; treating the mixture with a liquid solution to obtain a mass; forcing the mass through a screen having openings of a predetermined size; drying the wet granules on trays in drying machines, and the like; and re-grinding and re-screening to obtain granules having suitable sizes such as those used for compression into tablets. Examples of equipment required for such methods typically include fluidized bed granulators, rapid mixing granulators, planetary mixers, multi mills, cone blenders and high shear mixers, among others, as well as supporting utility systems. The number and variety of equipment typically requires a large space for operation with current good manufacturing standards, which requires further expense. Operation and maintenance of such machinery is also, tedious, labor intensive, and costly.

In addition, wet granulation methods may not be suitable for active agents which are sensitive to moisture or solvent and drying at either high or even low temperatures for a required duration. Incompatibilities between formulation ingredients can be aggravated by the granulating solvent. In addition, there is a possibility of material loss during processing due to the transfer of material from one operating unit to another. The dissolution rate of tablets manufactured by wet granulation may change-increase or decrease upon storage, and it is often difficult to obtain a formulation with desired granule hardness. Consequently, tablets or other products with granules falling outside a desired degree of hardness (e.g. too hard) can result in poor dissolution and/or disintegration of tablets when contacted with saliva or water in the mouth.

Dry granulation methods include mixing ingredients, roller compacting or slugging, dry screening or milling the mix to a course dry granulate, lubricating, and then compressing the lubricated granules. In dry granulation, the ingredients are not exposed to moisture, solvents and extreme heat. Thus, dry granulation can be used to process moisture, solvent and/or heat sensitive active ingredients. Often, the material to be tableted is compressed to a large mass, or “slug,” which is converted to tablets by a second compression process. Because slugging is a slow and uneconomic process, roller compaction has become the method of choice for dry granulation. Dry granulation requires specialized heavy-duty equipment. Moreover, with dry granulation methods it is often difficult to control the size of the resultant granules and loss of starting material is usually greater with dry granulation than with other methods. The dry granulation process also produces significant amounts of dust, which represents loss of materials, and may cause a hazard to equipment and personnel and cross contamination. Dry granulation, also suffers from a number of issues, such as segregation of components post mixing, and powder flow. Tablets manufactured by dry granulation tend to be softer than those manufactured by wet granulation, rendering them more difficult to process using post-tableting techniques.

Granulation can also be performed by a spray drying process. However, the main disadvantage of spray drying is the high capital investment, high heat consumption and high operational costs. Further, the most common feed materials in spray-drying processes are aqueous-based solutions, emulsions and suspensions, where water is evaporated in the dryer. A further disadvantage of spray drying is the limited particle size, which varies within the range of about 70-100 μm.

Another granulation technique is Extrusion/Spheronization. Extrusion-spheronization techniques are the most popular method of producing pellets or spheroids. Spheronized products are relatively dense and uniform size and shape. This process is especially useful for controlled-release solid oral dosage forms with a minimum number of excipients. For extrusion, different extruders such as screw extruders, sieve extruders, basket extruders, roll extruders, ram extruders, etc., are used to form extrudates. In spheronization, the extruded, cylindrically shaped segments are broken into uniform lengths and are gradually transformed into individual spherical shapes. The requirements of a multi-step batch process, as well as time and labor intensity, high initial setup cost, long production time, and process limitations, as well as inconsistent product results represent major drawbacks of extrusion/spheronization.

Melt granulation (also defined as thermoplastic granulation) operates via similar principles as wet granulation. However, instead of a binder solution this technique employs a molten binder infiltrate. The process is usually used for sustained, modified and targeted release capabilities, but requires high energy input and occurs in the absence of water. One significant drawback to melt granulation is its complicated nature and the large number of processing factors which affect the product outcome. Process related factors include binder spray rate, atomization air pressure, fluidizing air flow and inlet process air temperature; equipment related factors include shaker cycle, nozzle & nozzle height, container & chamber design, air distribution plate, and formulation related factors include low dose drug content, properties of starting material at low density, small particle size, lack of stickiness etc. Thus, due to these and other factors melt granulation is complicated, process sensitive and multi-step, requires significantly diverse and expensive equipment, and is prone to operation errors which reduce production reliability and product consistency.

Another granulation technique is solid dispersion which can be used for solubility enhancement of hydrophobic or lipophilic crystalline drugs. In the first generation of solid dispersions, crystalline carriers (i.e. mostly small molecular additives) were used for dispersing the drug homogeneously in the solid state. However, this has the disadvantage that, a rather fast drug precipitation upon aqueous dispersion often occurred. Therefore, a second generation of solid dispersion was based on polymeric carriers. In this generation a dissolution rate that was widely controlled by the hydration and dissolution of the polymeric matrix was achieved. A third-generation solid dispersion evolved the process by employing polymeric carriers with surfactants to improve the in-vivo aqueous dispersion following oral administration of the product. Selection of the manufacturing method based upon the physicochemical drug properties is therefore always desirable.

The present inventors have recognized an unmet need for a simple process that can agglomerate a wide variety of solid particulate material irrespective of their specific properties, such as solubility, density, reactivity, and form in the creation of various compositions and dosage forms that are consistent, inexpensive, stable upon storage, and accommodate multiple final forms with customizable properties.

### SUMMARY OF THE DISCLOSURE

Aspects of the present disclosure relate to a unique, rapid and highly efficient method to articulate (e.g. agglomerate) materials into Bonded MicroParticulates (BMPX) of a desired size and character, which is capable of treating a wide variety of solid particulate starting materials regardless of their specific forms and particular properties—in particular, regardless of their solubility in aqueous or nonaqueous medium, morphology (e.g., amorphous or crystalline), particle density, particle size, particle shape, reactivity, hydrophilicity, hydrophobicity or stability. The solid particulate starting material may be a therapeutic ingredient such as a pharmaceutically active ingredient (e.g., anti-diabetic agent, anti-hypertensive agent) or a bioactive agent, a dietary supplement (e.g., vitamin, fiber, etc.), a non-active ingredient useful as an excipient in the preparation of pharmaceutical dosage forms (e.g., carriers, diluents), a food ingredient, Mannitol, Xylitol, Sugar, dicalcium phosphate or combination of ingredients (e.g., ingredients used in dry beverage mix, food flavoring excipient, food additive), an oral care agent, an agrochemical, or an animal food. The present disclosure takes advantage of selecting simple material(s), reproducible consistent processes, and consistent techniques to produce Bonded MicroParticulates (BMPX) of a wide variety of Actives, Excipients and Inactive ingredients regardless of their specific forms and particular properties, thus eliminating the use organic solvents, excessive heat, expensive equipment, utility systems, space, good manufacturing practices-Standard operating procedures, protocols, Validations and qualifications, Quality control tests, Labor and overhead costs, and time- and cost-consuming process steps. Specifically, the methods disclosed herein eliminate the disadvantages present with wet granulation, fluid bed processes, dry granulation spray granulation techniques, melt granulation, and many other known processes, such a lipid excipient techniques.

One aspect of the present disclosure provides compositions, processes and applications of high percentage solid particulate materials comprising: a) high percentage solid particulate material by weight, based on a total weight of the bonded microparticulates, selected from the group of pharmaceutically active ingredient, a bioactive agent, oral care agent, a dietary supplement, a pharmaceutical excipient, a food ingredient, an agrochemical, and an animal food in different forms as Amorphous, Crystalline or combination thereof, material with different solubilities and different particle densities and; b) low percentage of Bonding agents by weight, based on a total weight of the bonded microparticulates, selected from the group of polymers, emulsifiers, fats and combinations thereof; c) to form bonded microparticles, which can further be used to manufacture different dosage forms.

Another aspect of the present disclosure provides methods for preparing Bonded MicroParticulates (BMPX) of a solid particulate material, which comprises: a) softening a bonding at a temperature between room temperature and the melting point of the bonding agent b) contacting the solid particulate material with the softened Bonding agent to form a mixture comprising the solid particulate material and the Bonding agent; (c) Bonding agent converts solid particulate material-actives and excipients into BMPX by bonding the particles of desired particle size, and c) cooling the mixture to a predetermined temperature to produce bonded microparticulates. The method prepares bonded microparticulates either in the absence or presence of added water or other solvents.

In various embodiments, the bonding agent can be a polymer, an emulsifier, a fat, or a combination thereof.

In certain embodiments, the solid particulate starting material is a pharmaceutically active ingredient. In certain embodiments, the solid particulate starting material is a dietary supplement. In certain embodiments, the solid particulate starting material is pharmaceutical excipient such as a diluent. In certain embodiments, the solid particulate starting material is oral care agent.

Bonded microparticulates produced by the any other method are also deemed to be within the scope of the present disclosure. The bonded microparticulates, in accordance with the present disclosure, to be designated without distinction also by the term solid agglomerated mass or solid agglomerated particles, have great versatility in application, and can be used in many medical and consumer products for human and animal use, such as pharmaceutical dosage forms (e.g., tablets, capsules, dry injectables, rapid-melt tablets, chew tablets, rapid-melt beads, topical compositions), biologics (e.g., vaccines), dietary supplements, food products, dry beverages, confectionery and animal food.

In an embodiment, the present disclosure is directed to a pharmaceutical composition comprising the bonded microparticulates of the present disclosure, wherein the bonded microparticulates comprise a pharmaceutical active ingredient and a bonding agent.

In another embodiment, the present disclosure is directed to a tablet comprising the bonded microparticulates of the present disclosure, wherein the bonded microparticulates comprise an active ingredient (pharmaceutical or dietary active ingredient or oral care agent) and a bonding agent. Advantageously, the bonded microparticulate disclosed herein are capable of being compressed into tablets that exhibit desirable hardness, friability, disintegration time as per USP standards. The methods disclosed herein prepare bonded microparticulates that have excellent compressibility and processability, which prevents sticking of compressed tablet formulation to pressing dies and punches.

Various objects, features, aspects and advantages of the inventive subject matter will become more apparent from the following detailed description of preferred embodiments.

### BRIEF DESCRIPTION OF THE FIGURES

The accompanying figures are included to provide a further understanding of the present disclosure, and are incorporated in and constitute a part of this specification. The figures illustrate exemplary embodiments of the present disclosure and together with the description serve to explain the principles of the present disclosure

FIG. 1 Comparison of particle sizes by sieve analysis for BMPX of Metformin HCl with Bonding Agents A, B, C and D.

FIG. 2 shows the microscopic image of Fine Powder Metformin HCl.

FIG. 3 shows the microscopic image of BMPX comprising Fine Powder Metformin HCl and Bonding Agent A.

FIG. 4 shows the microscopic image of BMPX comprising Fine Powder Metformin HCl and Bonding Agent B.

FIG. 5 shows the microscopic image of BMPX comprising Fine Powder Metformin HCl and Bonding Agent C.

FIG. 6 shows the microscopic image of BMPX comprising Fine Powder Metformin HCl and Bonding Agent D.

FIG. 7 shows comparison of particle size by sieve analysis for BMPX of Niacin with Bonding Agents A, B, C.

FIG. 8 shows the microscopic image of Fine Powder Niacin.

FIG. 9 shows the microscopic image of Niacin BMPX comprising Fine Powder Niacin and Bonding Agent A.

FIG. 10 shows the microscopic image of Niacin BMPX comprising Fine Powder Niacin and Bonding Agent B.

FIG. 11 shows the microscopic image of Niacin BMPX comprising Fine Powder Niacin and Bonding Agent C.

FIG. 12 shows the comparison of dissolution data of Niacin BMPX 100 mg tablets with Bonding Agent A, B, C in 1.2 pH Buffer as dissolution medium.

FIG. 13 shows comparison of particle size by sieve analysis for BMPX of Guaifenesin with Bonding Agents A, B, C, D.

FIG. 14 shows the microscopic image of Fine Powder Guaifenesin.

FIG. 15 shows the microscopic image of BMPX comprising Fine Powder Guaifenesin and Bonding Agent A.

FIG. 16 shows the microscopic image of BMPX comprising Fine Powder Guaifenesin and Bonding Agent B.

FIG. 17 shows the microscopic image of BMPX comprising Fine Powder Guaifenesin and Bonding Agent C.

FIG. 18 shows the microscopic image of BMPX comprising Fine Powder Guaifenesin and Bonding Agent D.

FIG. 19 shows comparison of particle size by sieve analysis for BMPX of Amlodipine Besylate with Bonding Agents A, B.

FIG. 20 shows the microscopic image of Fine Powder Amlodipine Besylate.

FIG. 21 shows the microscopic image of BMPX comprising Fine Powder Amlodipine Besylate and Bonding Agent A.

FIG. 22 shows the microscopic image of BMPX comprising Fine Powder Amlodipine Besylate and Bonding Agent B.

FIG. 23 shows the comparison of dissolution data of BMPX Amlodipine Besylate 10 mg tablets with Bonding Agent A, B in 1.2 pH Buffer as dissolution medium.

FIG. 24 shows comparison of particle size by sieve analysis for BMPX of Caffeine Anhydrous with Bonding Agents A, B, C.

FIG. 25 shows the microscopic image of Fine Powder Caffeine Anhydrous.

FIG. 26 shows the microscopic image of BMPX comprising Fine Powder Caffeine Anhydrous and Bonding Agent A.

FIG. 27 shows the microscopic image of BMPX comprising Fine Powder Caffeine Anhydrous and Bonding Agent B.

FIG. 28 shows the microscopic image of BMPX comprising Fine Powder Caffeine Anhydrous and Bonding Agent C.

FIG. 29 shows comparison of particle size by sieve analysis for BMPX of Diphenhydramine HCL with Bonding Agents A, B, C

FIG. 30 shows the microscopic image of Crystalline Diphenhydramine HCL.

FIG. 31 shows the microscopic image of BMPX comprising Crystalline Diphenhydramine HCL and Bonding Agent A.

FIG. 32 shows the microscopic image of BMPX comprising Crystalline Diphenhydramine HCL and Bonding Agent B.

FIG. 33 shows the microscopic image of BMPX comprising Crystalline Diphenhydramine HCL and Bonding Agent C

FIG. 34 Comparisons of particle sizes by sieve analysis for BMPX of Ibuprofen with Bonding Agents A.

FIG. 35 shows the microscopic image of Fine Powder Ibuprofen.

FIG. 36 shows the microscopic image of BMPX comprising Fine Powder Ibuprofen and Bonding Agent A

FIG. 37 shows comparison of particle size by sieve analysis for BMPX of Sodium Bicarbonate with Bonding Agents A, B, C.

FIG. 38 shows the microscopic image of Crystalline Sodium Bicarbonate.

FIG. 39 shows the microscopic image of BMPX comprising Crystalline Sodium Bicarbonate and Bonding Agent A.

FIG. 40 shows the microscopic image of BMPX comprising Crystalline Sodium Bicarbonate and Bonding Agent B.

FIG. 41 shows the microscopic image of BMPX comprising Crystalline Sodium Bicarbonate and Bonding Agent C.

FIG. 42 shows comparison of particle size by sieve analysis for BMPX of Zinc Gluconate with Bonding Agents A, B.

FIG. 43 shows the microscopic image of Fine Powder Zinc Gluconate.

FIG. 44 shows the microscopic image of BMPX comprising Fine Powder Zinc Gluconate and Bonding Agent A.

FIG. 45 shows the microscopic image of BMPX comprising Fine Powder Zinc Gluconate and Bonding Agent B.

FIG. 46 shows comparison of particle size by sieve analysis for BMPX of Fructo-Oligosaccharide with Bonding Agents A, B.

FIG. 47 shows the microscopic image of Fine Powder Fructo-Oligosaccharide.

FIG. 48 shows the microscopic image of BMPX comprising Fine Powder Fructo-Oligosaccharide and Bonding Agent A.

FIG. 49 shows the microscopic image of BMPX comprising Fine Powder Fructo-Oligosaccharide and Bonding Agent B.

FIG. 50 shows comparison of particle size by sieve analysis for BMPX of Calcium carbonate with Bonding Agents A, B, C, D.

FIG. 51 shows the microscopic image of Fine powder Calcium Carbonate.

FIG. 52 shows the microscopic image of BMPX comprising Fine Powder Calcium Carbonate and Bonding Agent A.

FIG. 53 shows the microscopic image of BMPX comprising Fine Powder Calcium Carbonate and Bonding Agent B.

FIG. 54 shows the microscopic image of BMPX comprising Fine Powder Calcium Carbonate and Bonding Agent C.

FIG. 55 shows the microscopic image of BMPX comprising Fine Powder Calcium Carbonate and Bonding Agent D

FIG. 56 shows comparison of particle size by sieve analysis for BMPX of Xylitol-300 with Bonding Agents A, B, C.

FIG. 57 shows the microscopic image of Crystalline Xylitol-300.

FIG. 58 shows the microscopic image of BMPX comprising Crystalline Xylitol-300 and Bonding Agent A.

FIG. 59 shows the microscopic image of BMPX comprising Crystalline Xylitol-300 and Bonding Agent B.

FIG. 60 shows the microscopic image of BMPX comprising Crystalline Xylitol-300 and Bonding Agent C.

FIG. 61 shows Comparison of particle sizes by sieve analysis for BMPX of Xylitol-90 with Bonding Agents A, B, C, D.

FIG. 62 shows the microscopic image of Fine Powder Xylitol-90.

FIG. 63 shows the microscopic image of BMPX comprising Fine Powder Xylitol-90 and Bonding Agent A.

FIG. 64 shows the microscopic image of BMPX comprising Fine Powder Xylitol-90 and Bonding Agent B.

FIG. 65 shows the microscopic image of BMPX comprising Fine Powder Xylitol-90 and Bonding Agent C.

FIG. 66 shows the microscopic image of BMPX comprising Fine Powder Xylitol-90 and Bonding Agent D

FIG. 67 shows comparison of particle size by sieve analysis for BMPX of Sugar powder with Bonding Agents A, B, C, D.

FIG. 68 shows the microscopic image of Crystalline Sugar Powder.

FIG. 69 shows the microscopic image of BMPX comprising Crystalline Sugar Powder and Bonding Agent A.

FIG. 70 shows the microscopic image of BMPX comprising Crystalline Sugar Powder and Bonding Agent B.

FIG. 71 shows the microscopic image of BMPX comprising Crystalline Sugar Powder and Bonding Agent C.

FIG. 72 shows the microscopic image of BMPX comprising Crystalline Sugar Powder and Bonding Agent D

FIG. 73 shows comparison of particle size by sieve analysis for BMPX of Mannitol with Bonding Agents A, B, C, D.

FIG. 74 shows the microscopic image of Fine Powder Mannitol.

FIG. 75 shows the microscopic image of BMPX comprising Fine Powder Mannitol and Bonding Agent A.

FIG. 76 shows the microscopic image of BMPX comprising Fine Powder Mannitol and Bonding Agent B.

FIG. 77 shows the microscopic image of BMPX comprising Fine Powder Mannitol and Bonding Agent C.

FIG. 78 shows the microscopic image of BMPX comprising Fine Powder Mannitol and Bonding Agent D.

FIG. 79 shows comparison of particle size by sieve analysis for BMPX of Dibasic Calcium Phosphate Anhydrous with Bonding Agents A.

FIG. 80 shows the microscopic image of Fine Powder Dibasic Calcium phosphate Anhydrous.

FIG. 81 shows the microscopic image of BMPX comprising Fine Powder Dibasic Calcium phosphate anhydrous and Bonding Agent A.

FIG. 82 shows comparison of particle size by sieve analysis for BMPX of Lactose Anhydrous with Bonding Agents A.

FIG. 83 shows the microscopic image of Fine Powder Lactose Anhydrous.

FIG. 84 shows the microscopic image of BMPX comprising Fine Powder Lactose anhydrous and Bonding Agent A.

It should be noted that all figures illustrating an image of a powder, grannules, or BMPX are imaged at a 300×level of magnification utilizing the same equipment for purposes of scale uniformity comparison.

### DETAILED DESCRIPTION OF EMBODIMENTS

Before disclosure embodiments are described, it is to be understood that this disclosure is not limited to the particular structures, process steps, or materials disclosed herein, but is extended to equivalents thereof as would be recognized by those ordinarily skilled in the relevant arts. It should also be understood that terminology employed herein is used for the purpose of describing particular examples or embodiments only and is not intended to be limiting.

Furthermore, the described features, structures, or characteristics can be combined in any suitable manner in one or more embodiments. In the following description, numerous specific details are provided, such as examples of compositions, dosage forms, treatments, etc., to provide a thorough understanding of various disclosure embodiments. One skilled in the relevant art will recognize, however, that such detailed embodiments do not limit the overall disclosure concepts articulated herein, but are merely representative thereof.

### Definitions

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

In this disclosure, “comprises,” “comprising,” “containing” and “having” and the like can have the meaning ascribed to them in U.S. Patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the compositions nature or characteristics would be permissible if present under the “consisting essentially of” language, even though not expressly recited in a list of items following such terminology. When using an open-ended term, like “comprising” or “including,” in this written description it is understood that direct support should be afforded also to “consisting essentially of” language as well as “consisting of” language as if stated explicitly and vice versa.

The terms “first,” “second,” “third,” “fourth,” and the like in the description and in the claims, if any, are used for distinguishing between similar elements and not necessarily for describing a particular sequential or chronological order. It is to be understood that any terms so used are interchangeable under appropriate circumstances such that the embodiments described herein are, for example, capable of operation in sequences other than those illustrated or otherwise described herein. Similarly, if a method is described herein as comprising a series of steps, the order of such steps as presented herein is not necessarily the only order in which such steps may be performed, and certain of the stated steps may possibly be omitted and/or certain other steps not described herein may possibly be added to the method.

Reference throughout this specification to “one embodiment” or “an embodiment” or to “one example,” or “an example,” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

In some embodiments, the numbers expressing quantities of ingredients, properties such as concentration, process conditions, and so forth, used to describe and claim certain embodiments of the invention are to be understood as being modified in some instances by the term “about.” Accordingly, in some embodiments, the numerical parameters set forth in the written description are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. For example, for the sake of convenience and brevity, a numerical range of “about 50 angstroms to about 80 angstroms” should also be understood to provide support for the range of “50 angstroms to 80 angstroms.” Furthermore, it is to be understood that in this specification support for actual numerical values is provided even when the term “about” is used therewith. For example, the recitation of “about” 30 should be construed as not only providing support for values a little above and a little below 30, but also for the actual numerical value of 30 as well.

As used herein, comparative terms such as “increased,” “decreased,” “better,” “worse,” “higher,” “lower,” “enhanced,” “improved,” “maximized,” “minimized,” and the like refer to a property of a device, component, composition, biologic response, biologic status, or activity that is measurably different from other devices, components, compositions, biologic responses, biologic status, or activities that are in a surrounding or adjacent area, that are similarly situated, that are in a single device or composition or in multiple comparable devices or compositions, that are in a group or class, that are in multiple groups or classes, or as compared to an original (e.g. untreated) or baseline state, or the known state of the art. For example, a formulation having a “higher” or “lower” concentration of particles can have a number of particles that is greater than or lower than a number of particles in comparable formulation having a substantially same size, mass, or volume, and which may contain substantially the same ingredients. Such comparable formulation may be expressly provided for direct comparison, or generally known in the prior art.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, levels and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges or decimal units encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

Units of measure of amounts or concentrations can be expressed herein by any suitable and recognized quantitation or output, such as milligrams (mg), milliliters (ml), etc. Individual ingredients or agents can also be expressed in relation to other ingredients or agents, or combinations of ingredients or agents, such as a composition or formulation. For example, the concentration or amount of an ingredient or agent can be articulated in terms of its percentage by weight (e.g. weight percent, or percent weight, wt %) of the composition or formulation. Unless the context dictates otherwise, when using terms such as wt % in this disclosure, the amount of the identified ingredient or agent will be its percentage by weight of the composition or formulation, or other sub-formulation or mixture combination identified. Additionally, numerical values expressed in terms of ratios will be in terms of weight percentage (wt %) ratios unless otherwise state expressly or by context.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

As used herein, an “effective amount” or a “therapeutically effective amount” of a drug refers to a non-toxic, but sufficient amount of the drug, to achieve therapeutic results in treating a condition for which the drug is known to be effective. It is understood that various biological factors may affect the ability of a substance to perform its intended task. Therefore, an “effective amount” or a “therapeutically effective amount” may be dependent in some instances on such biological factors. Further, while the achievement of therapeutic effects may be measured by a physician or by other qualified medical personnel using evaluations known in the art, it is recognized that individual variation and response to treatments may make the achievement of therapeutic effects a somewhat subjective decision. The determination of an effective amount is well within the ordinary skill in the art of pharmaceutical sciences and medicine.

As used herein, the terms “formulation” and “composition” are used interchangeably and refer to a mixture of two or more compounds, elements, or molecules. In some aspects, the terms “formulation” and “composition” may be used to refer to a mixture of one or more active agents with a carrier or other excipients. Furthermore, the term “dosage form” can include one or more formulation(s) or composition(s) provided in a format for administration to a subject. For example, an “oral dosage form” can be suitable for administration to a subject's mouth. A “topical dosage form” can be suitable for administration to a subject's skin by rubbing, etc.

As used herein, the terms “release” and “release rate” are used interchangeably to refer to the discharge or liberation of a substance, including without limitation a drug, from the dosage form into a surrounding environment such as an aqueous medium either in vitro or in vivo.

As used herein, “cooling” refers to that act of reducing the substance's temperature. When the substance is at a temperature above ambient or room temperature, the act of cooling can be passively allowing the temperature of the substance to reduce toward ambient or room temperature, or actively taking steps to facilitate a temperature reduction, including agitation, refrigeration, etc. Furthermore, substances can be cooled below ambient or room temperature by employing known mechanisms to further reduce the temperature, such as cold-water baths, icing, refrigeration, freezing, etc.

The term “pharmaceutically acceptable” as used herein refers to compounds, materials, compositions, and/or dosage forms which are, within the scope of sound medical judgment, suitable for use in contact with tissues of human beings and animals and without excessive toxicity, irritation, allergic response, or any other problem or complication, commensurate with a reasonable benefit/risk ratio.

The terms “therapeutic agent,” “active agent,” “pharmaceutically active”, “active pharmaceutical ingredient”, “active drug” and “drug” as used herein are used interchangeably and are defined to mean any agent or substance such as an active pharmaceutical ingredient (“API”) that has measurable specified or selected physiologic activity when administered to a subject in a significant or effective amount. When these terms are used, or when a particular active agent is specifically identified by name or category, it is understood that such recitation in this written description is intended to include the active agent per se, as well as provide express support for pharmaceutically acceptable salts, polymorphs, prodrugs, as well as the anhydrous, hydrated, and solvated forms thereof, esters thereof, or compounds significantly related thereto, including without limitation, prodrugs, active metabolites, isomers, individually optically active enantiomers thereof, and the like. Moreover, when a specific form of a compound such as a salt, ester, prodrug, metabolite, isomer, etc., is recited in this written description, it is to be understood that such recitation also provides express support for the active agent per se (e.g. free base) or other well-known forms of the active agent and vice versa.

The term “oral dosage form” as used herein is defined to mean a dosage form which is administered by mouth, for transmucosal absorption through the mucous membranes of the mouth and/or, enteral absorption after swallowing, through the gastrointestinal tract. Such oral dosage forms include but are not limited to tablets, buccal dosage forms and sublingual dosage forms suitable for oral administration.

The term “immediate release dosage forms” or dosage forms which exhibit an “immediate release” of active drug as used herein is defined to mean dosage forms which provide a substantially immediate rate of release of active drug. Immediate release dosage forms typically release drug content into gastrointestinal tract within a short period of time after administration, and plasma drug levels generally peak shortly after dosing.

In some aspects of the present disclosure, the release of the drug may be controlled release. As used herein, the term “controlled release” represents the release of the drug from the dosage form according to a profile that differs from an unrestricted or uncontrolled release profile, such as a predetermined profile. In some aspects, the controlled release selected can be, intermediate, delayed, extended, sustained, or pulsatile. In another aspect, combinations of the aforementioned release profiles may be used in order to achieve specific delivery results, such as an immediate release followed by a delayed and/or a sustained release of the active agent.

The term “dissolution profile” or “release profile” as used herein are used interchangeably in this disclosure, and are defined to mean a quality control test conducted according to instructions found in the United States Pharmacopoeia (“USP”), i.e. using a USP apparatus design with a dissolution medium as found in the USP. Dissolution tests in-vitro measure the rate and extent of dissolution of active drug in an aqueous dissolution medium. The terms “% released” and “% dissolved”, when referring to a dissolution profile, are used interchangeably in this application and are defined to mean the extent (%) of active drug released in an aqueous dissolution medium (in vitro).

The term “tablet” as used herein refers to a single dosage form, i.e. a single entity containing active ingredients that is administered to the subject. The term “tablet” also includes a tablet that may be the combination of one or more “minitablets” or tablet in a tablet, tablet with layers or coatings, etc.

As used herein, the terms “solid particulate material” and “solid particulate starting material” are interchangeable. Solid particulate material can be a pharmaceutically active material, inactive material, dietary supplement, food ingredient, oral care agent or an agrochemical material. It is available as any type of solid substance in granular, powder or particulate form, depending on the nature of the substance and its use. As used herein, an “edible” solid particulate material refers to a material that is safe for consumption by an animal, such as a human, and may have therapeutic, nutritional, or other positive health imparting effects on the animal. Examples of edible solid particulate materials include without limitation, pharmaceutically active materials and inactive materials, dietary supplements, food ingredients, herbal or botanical extractions, and oral care agents. Solid particulate material used herein may be single material or combination of materials. A “high” percentage of solid particulate material can mean that the solid particulate material is present in an amount of about 70% by weight or more based on a total weight of the bonded microparticulates (BMPX). In some embodiments, the amount can be more than about 75%, more than about 80%, more than about 90%, or more than about 95% by weight of the BMPX (e.g. from about 70% to about 98% by weight).

As used herein, the term “bonding agent” refers to a substance or material that is capable of engaging and holding solid particulate materials together in an agglomeration. A bonding agent is in a solid, semi-solid, or liquid state at room temperature, and softens when heated to a temperature below the melting point thereof. Bonding agents also soften when heated to a temperature below a melting point (or melting range) of a solid particulate material with which the bonding agent is combined or is to be combined. Once combined or mixed with the solid particulate materials, the bonding agent forms agglomerates by holding the solid particulate materials together with various forces, such as cohesion, adhesion, etc. A bonding agent is not a solvent (though in some embodiments, it may include a small amount of water or other agent, such as glycerin or propylene glycol as an auxiliary component), nor is it used as dry powder in the blend or used as a granulating solution. A bonding agent is not an excipient such as a binder, lubricant, etc., used in the traditional sense of a finished pharmaceutical product or dosage formulation, such as a tablet. However, such ingredients may be used once the BMPX agglomeration material has been formed in order to create compositions, products, and dosage forms from the BMPX material. As used herein, a “low” percentage of a bonding agent means that the bonding agent is used in an amount up to about 50% by weight based on a total weight of the bonded microparticulates. In some embodiments, the bonding agent can be present in an amount of below about 30% by weight, 20% by weight, 18% by weight, 12% by weight, or 10% by weight (e.g. from 50% by weight down to 10% or even lower, 5% or 3% by weight or 1% by weight).

As used herein, the term “Bonded MicroParticulate” (BMPX) refers to microparticulate wherein, the bonding agent and particles of the solid particulate material are in contact with one another, such as direct contact, and are closely bound together via one or more forces, such as adhesion, cohesion, tension, etc. into an agglomeration. The bonded microparticulate has a size larger than the original particle size of the solid particulate material with special properties such as free flow, improved compressibility, low friability, good active release, improvement of properties such as surface texture, porosity or wettability, positive influence on the disintegration time and the solubility of the active substance, prevents segregation of components, reduces the level of dust present during manufacturing process thereby reducing the incidence of cross-contamination and risk to workers.

Other terms are defined as they appear in the following description and should be construed in the context with which they appear.

### DESCRIPTION

Reference will now be made in detail to embodiments of the disclosure. While the disclosure will be described in conjunction with the embodiments, it will be understood that it is not intended to limit the disclosure to those embodiments. To the contrary, it is intended to cover alternatives, variants, modifications, and equivalents as may be included within the spirit and scope of the disclosure as defined by the appended claims.

An initial overview of technology embodiments is provided below, and then specific technology embodiments are described in further detail later. This initial summary is intended to aid readers in understanding the technology more quickly but is not intended to identify key features or essential features of the technology nor is it intended to limit the scope of the claimed subject matter.

As discussed above, there is no one type of formulation approach which can be used for wide variety of solid particulate material. Selection of the most appropriate granulation process is important because the consequences can be significant with respect to the performance of the dosage form. Solid particulate material can be hydrophilic or hydrophobic or lipophilic in nature. They can also be divided based on their form, as crystalline and amorphous. They can further be divided based on density of the material as low-density material, medium density material and high-density material.

Physical characteristics like the crystalline and amorphous states of solid substance significantly influence the various important properties like solubility, dissolution rate, and bioavailability of the drug. An amorphous state is generally more soluble than a crystalline state, however there is a problem of stability of amorphous solid particulate matter. Sometimes due to the insoluble nature of the crystalline state, the solid particulate material needs to be converted to an amorphous state when manufacturing dosage form.

Further, low aqueous solubility is the major problem encountered with formulation development as any solid particulate matter to be absorbed must be present in the form of an aqueous solution at the site of absorption. Poor aqueous solubility, and consequently also poor dissolution rate, is a major challenge specifically in the systemic delivery of orally administered drugs. High aqueous solubility of the active pharmaceutical needs a different strategy to manufacture the dosage form.

Further, density is an important property of pharmaceutical compounds from powder (drug or excipient) to tablet. Bulk and tapped density are related to flow properties of powder. The flowability of pharmaceutical blends (i.e., API plus excipients) is important for effective use of direct compression. Poor flow can cause bridging, arching, surging, and enhanced movement of particles in the die cavity. Usually, bulk density is of great importance when one considers the size of a high-dose tablet product or the homogeneity of a low-dose formulation in which there are big differences in drug and excipients densities.

Solid particulate materials that are good to sparingly soluble in water and amorphous can be granulated with either wet or dry methods. For material with poor flowability and compressibility, fluid-bed granulation remains a viable choice, while materials that are sensitive to moisture but stable at higher temperatures can be granulated using melt granulation. For insoluble and highly crystalline drugs with poor solubility, melt granulation and/or spray-drying granulation may be preferred. Slugging is an option for APIs with poor flowability that are also highly sensitive to hydrolysis, but fluctuations in the forces applied to the individual slugs can lead to variability in the particle size of the granules and, hence, result in reduced content uniformity. Finally, for material that will be formulated into tablets with taste masking and/or controlled release profiles, fluid-bed granulation is widely used because it enables the formation of coating layers.

As discussed above, there is no one process of preparing compositions or dosage forms which are applicable to all types of solid particulate matter and much consideration needs to be given to solubility, density and form before picking one of the techniques for preparing a composition or dosage form. Identifying any special techniques or methods to ensure a robust granulation method for preparing a given dosage form is often challenging. Thus, there is an long-felt and unmet need for compositions and methods/processes to create unique, less complicated formulations, reduce regulatory burdens, reduce the burden of standard operating procedures and validations, that are reproducible and consistent, cost effective, substantially eliminate multistep processes, reduced use of expensive equipment, are applicable to varied type of solid particulate material irrespective of their form (e.g. crystalline or amorphous, differing densities and differing solubilities).

The inventors of the present invention have unexpectedly found unique formulations and processes to meet the challenge of these long-felt, and unmet needs, which can be used for a wide variety of solid particulate materials irrespective of solubility, form and density. In one embodiment, such a technique includes the preparation of bonded microparticulates comprising a solid particulate or powder material (e.g. an API) and one or more bonding agents. The invention of manufacturing bonded microparticulates is innovative, rapid and cost-effective for the formation of agglomerates of solid particulate material without using expensive equipment. The present invention avoids the need for specialty ingredients, multiple processes as required with wet or dry granulation or extrusion speronization or melt granulation or Self Emulsifying Drug Delivery System or spray granulation etc. techniques. The use of excessive processes or expensive excipients is also avoided. The output is faster and can be directly used to prepare various compositions and dosage forms. In some embodiments, the only substantive steps for forming a BMPX or agglomeration/granules can be heating, mixing, and cooling, for example, as demonstrated in the examples below.

The present invention unexpectedly allows bonded microparticulates comprising solid particulate material and bonding agent to be applied to a wide variety of solid particulate material of varying density, solubility and form. The bonded microparticulates so formed can be incorporated with disintegrants, lubricants, glidants or even directly compressed into tablets or filled into capsules. One more advantage of bonded microparticles is that they do not require any other processing step such as drying or milling. In other formulation techniques such as granulation or extrusion or Self Nano Emulsifying Drug Delivery System or Self Emulsifying Drug Delivery System or melt granulation etc., once the granules are formed, they must be further subjected to many downstream processes such as drying, milling, sieving to control particle size distribution.

The bonded microparticles of the present invention can be prepared using minimum processes and minimum inactive ingredients and have advantageous properties of being unique processes, ease of manufacturing, low cost, lack of use of expensive equipment and their maintenance, less space and manpower and lower cost of goods. The dosage forms have been found to have adequate friability, hardness, uniformity of weight, adequate disintegration time and dissolution rate, no change in moisture content from actives and have abundant utility over the known products.

The present disclosure provides a rapid and highly efficient method for converting solid particulate material, into bonded microparticulates of desired size and character, which is capable of treating a wide variety of solid particulate starting materials regardless of their specific forms whether amorphous or crystalline, solubility, whether hydrophilic or hydrophobic or lipophilic and density, whether low-density material, medium-density material and high-density material. The solid particulate starting material may be a therapeutic ingredient such as a pharmaceutically active ingredient (e.g., anti-diabetic agent, anti-hypertensive agent) or a bioactive agent, dietary supplement (e.g., Vitamin, Fiber), a non-active ingredient useful as excipient in the preparation of pharmaceutical dosage forms (e.g., carriers, diluents), a food ingredient (e.g., ingredients used in dry beverage mix, food flavoring excipient, food additive), an oral care agent, an agrochemical, or an animal food. The present invention contemplates single therapeutic ingredient or as well as combination of solid particulate material.

One embodiment of the present invention discloses composition, process and applications of high percentage solid particulate materials comprising (a) high percentage solid particulate material by weight, based on a total weight of the bonded microparticulates, selected from the group of pharmaceutically active ingredient, a bioactive agent, oral care agent, a dietary supplement, a pharmaceutical excipient, a food ingredient, an agrochemical, and an animal food in different forms as amorphous, crystalline or combination thereof, material with different solubilities and different particle densities and (b) low percentage of Bonding agents by weight, based on a total weight of the bonded microparticulates, selected from the group of polymers, emulsifiers, fats and combination s thereof (c) to form bonded microparticles, which can further be used to manufacture different dosage forms.

Another embodiment of the present invention discloses Bonded Microparticulates comprising high percentage of solid particulate material and low percentage of bonding agent, the solid particulate material includes a wide variety of active and inactive ingredients regardless of their specific forms, solubility and density. The process of preparing the bonded microparticulates is equally applicable for crystalline solid particulate material, amorphous solid particulate material, low-density solid particulate material, medium density solid particulate material, high density solid particulate material, hydrophilic solid particulate material or hydrophobic or lipophilic solid particulate materials. One of the most important advantages of preparing bonded microparticulates over prior arts process is that it is applicable to each and every type of solid particulate materials. There is not one prior art method which can be used to formulate dosage form of solid particulate material with differing physical properties such as form whether amorphous or crystalline or combination, differing solubility whether hydrophilic, lipophilic or hydrophobic and differing densities whether low, medium or high density. Specifically, the method disclosed herein eliminates the disadvantages associated with wet granulation, dry granulation, fluid bed processing, spray granulation techniques as well as lipid-based formulation techniques such as melt granulation, self-emulsifying drug delivery systems etc.

In one embodiment, the present disclosure provides bonded microparticulates comprising high percentage of solid particulate material and low percentage bonding agent, by weight, based on the total weight of bonded microparticulates.

In another embodiment, the present disclosure provides a method for preparing bonded microparticulates of a solid particulate material, which comprises: a) softening the bonding agent at a temperature between room temperature and the melting point of the bonding agent, thereof to obtain a softened bonding agent b) contacting the solid particulate material with the softened bonding agent to form a mixture comprising the solid particulate material and the bonding agent; and c) cooling the mixture to a predetermined temperature to produce bonded microparticulates. Depending on the specific solid particulate material and the bonding agent material employed, the bonding agent can soften at a temperature between 30° C. to 100° C. Preferably, the temperature at which bonding agent softens is from about 40° C. to about 70° C. Preferably the bonding agent is softened when heated around a temperature of 60° C. depending upon the melting point of bonding agent.

In another embodiment, the present disclosure provides a method for preparing bonded microparticulates of a solid particulate material, which comprises: a) softening the bonding agent at a temperature between room temperature and the melting point of the bonding agent, thereof to obtain a softened bonding agent b) mixing the softened bonding agent with up to 3% of water or moisture c) contacting the solid particulate material with the softened bonding agent of step b) to form a mixture comprising the solid particulate material and the bonding agent; and c) cooling the mixture to a predetermined temperature to produce bonded micro particulates. Typically, the bonding agent softens at a temperature between 30° C. to 100° C. Preferably, the temperature at which bonding agent softens is from about 40° C. to about 70° C. Preferably the bonding agent softened when heated around a temperature of 60° C. depending upon the melting point of bonding agent. Sometime the temperature at which the bonding agent was softened, was maintained till the solid particulate material was mixed with it. The mixing or contacting time of softened bonding agent with solid particulate material can range from about 5 min to about 30 min, most preferably the mixing time can be about 8 min to about 20 min depending upon the properties of solid particulate material such as form, solubility and density.

Another embodiment of the present disclosure is that the present invention does not require a drying step or milling step to further process the bonded microparticulates. In all other prior art methods such as granulation, melt granulation, once the granules are formed, granules are subjected to a drying or milling or sizing step before final dosage form such as tablets or capsule can be prepared by mixing or blending with other inactive ingredients. In the present invention the bonded microparticulates so produced were blended with other inactive ingredients to manufacture dosage form. For eg the bonded microparticulates are mixed with other inactive ingredients such as disintegrants, diluents, lubricants or glidants and compressed into tablet without any drying or milling step.

The invention of the present invention subjects the solid particulate material to minimum steps and minimum exposure to high temperature. Thus, compared to prior art such as wet and dry granulation, melt granulation, solid dispersion, SEDDS or SNEDDS or SMEDDS etc. the present invention saves lot of time and cost for the manufacture the dosage forms.

The bonding agent is a solid or semi-solid material at room temperature, which softens when heated at a temperature between room temperature and the melting point of the bonding agent not exceeding the melting point (or melting range) of the solid particulate material. Molten or softened or semi-solid, viscous or liquid words are used synonymously with each other. Typically, the bonding agent softens at a temperature between 30° C. to 100° C. Preferably, the temperature at which bonding agent softens is from about 40° C. to about 70° C. Preferably the bonding agent softens when heated around a temperature of 60° C., depending upon the melting point of bonding agent. The mixing of the solid particulate material with the softened bonding agent can be performed at any temperature that is practical, preferably, the contacting is performed at a temperature of from about 30° C. to about 60 0° C. Preferably the temperature is maintained during the mixing of solid particulate material with the softened bonding agent depending upon the properties of solid particulate material as form, solubility and density. As used herein, the term “contacting” includes blending, mixing, massing, combining and the like of the ingredients. When the solid particulate material is contacted/mixed with the softened bonding agent, the softened bonding agent forms liquid bridges between the particles of the solid particulate material which change to solid bonds upon cooling. A solid microparticulate is thereby formed in which the bonding agent and the particles of the solid particulate material are closely bound together, forming bonded microparticulates. The cooling step may be performed either by simply exposing the mixture comprising the solid particulate material and the bonding agent to a lower temperature, e.g., room temperature or below room temperature (e.g., below 20° C., below 10° C., etc.). Cooling of the mixture may be carried out using a tray drier or fluidized bed drier. The cooling of the mixture results in the formation of bonded microparticulates.

One embodiment of the present invention requires that the process of preparing bonded microparticulates to take place in presence of water. The water is added to the bonding agent once the bonding agent has softened. The invention contemplates using up to about 3% of moisture or water. Further in place of water, solvent such as glycerine or propylene glycol or combination thereof can also be included. Another embodiment of present invention is that the bonded microparticulate is free or substantially free from any residual organic solvent. Free or substantially free of residual organic solvent means that the process of preparing bonded microparticulates does not require or otherwise utilize, any organic solvent as an intermediate step of the process and which is removed or substantially removed in a subsequent step of the process.

In another embodiment, the resultant bonded microparticulates have particle size different from that of the solid particulate matter. Bonded microparticulates as well as the solid particulate material were subjected to screen analysis The screen/sieve analysis was conducted as per USP42 NF-37 general chapter <786>. The bonded microparticulates were found to have coarser and larger particle size then the solid particulate material. They were free flowing, uniformly sized with uniform distribution of solid particulate material in the bonding agent and may be used directly as a product by compressing in tablet or filling in capsule, or blended with other ingredients to prepare a final blend and then compressed into tablets or filled into capsule. For example, to prepare tablets, certain excipients such as disintegrants, lubricants, glidants etc. may be added to the bonded microparticulates to further facilitate forming the tablet.

In addition to screen analysis, the tap density and the bulk density of the bonded microparticulates and solid particulate material were determined. The bulk density and tap density was determined according to per USP42 NF-37 general chapter <616>.

Further microscopic images of the bonded microparticulates and solid particulate material were taken. The microscopic images can be taken by any technique known in the art. The microscopic images of solid particulate material and bonded microparticulates clearly show that compared to solid particulate material, bonded microparticulates are coarser, larger, spherical uniformly distributed and does not require additional drying or milling step prior to admixing with other non-active ingredients. All the figures with microscopic images are captured using 300× magnification.

In various embodiments, the methods disclosed herein produce discrete free-flowing bonded microparticulates comprising bonded microparticulates and bonding agent.

According to embodiments of the present disclosure, the solid particulate (starting) material may be a therapeutic ingredient such as a pharmaceutically active ingredient or a bioactive agent, a dietary supplement, a non-active ingredient useful as excipient in the preparation of pharmaceutical dosage forms (e.g., carriers, diluents), a food ingredient (e.g., ingredients used in dry beverage mix, food flavoring excipient, food additive), oral care agent, an agrochemical, or an animal food. In various embodiments, the solid particulate material is present at high percentage in the composition. High percentage of solid particulate material mean that the solid particulate material is present in an amount more than 70% by weight based on a total weight of the bonded microparticulates preferably above about 75% by weight, based on a total weight of the bonded microparticulates, more preferably about above 80% by weight, based on a total weight of the bonded micro particulates.

These solid particulates (starting) material can be in Crystalline or Fine powder or Amorphous or Granular, powder or particulate form. The solid particulate material can be in any form powder or crystalline fine or coarse form, which is not subject to any limitation in terms of e.g., particle size, shape and density.

In some embodiments, the solid particulate starting material is in amorphous form.

In some embodiments, the solid particulate starting material is in crystalline form.

In some embodiments, the solid particulate starting material is in partially crystalline form.

In some embodiments, the solid particulate starting material is in partially amorphous form.

In some embodiments, the solid particulate starting material is a low-density solid particulate material having a bulk density of from about 0.1 to about 0.45 g/cm³.

In some embodiments, the solid particulate starting material is a medium-density solid particulate material having a bulk density of from about 0.5 to about 0.75 g/cm³.

In some embodiments, the solid particulate starting material is a high-density solid particulate material having a bulk density equal to or greater than 0.8 g/cm³.

In some embodiments, the solid particulate starting material has a bulk density in the range of about 0.1 to about 2.5 g/cm³.

In some embodiments, the solid particulate starting material is hydrophilic or hydrophobic or lipophilic in character. Solubility of drugs is measured by the amount of solvent needed to dissolve one part of the drug at a specific temperature. For example, a drug that is very soluble needs less than one part of solvent to dissolve one part of the drug. A drug that is considered freely soluble needs 1 to 10 parts of solvent, one that is soluble needs 10 to 30 parts of solvent, one that sparingly soluble need 30 to 100 parts of solvent, one that is slightly soluble needs 100-1,000 parts and one that is practically insoluble or insoluble needs more than 10,000 parts, per part of solute.

Drug solubility has one of the highest impacts on the desired concentration of a drug in systemic circulation, and therefore whether or not the drug will have the desired impact or pharmacological response in patients. Hydrophilic drugs exhibit good water solubility. According to the present invention, the term “hydrophilic material” is the opposite of a lipophilic material and refers to a drug that exhibits a certain degree of solubility in an aqueous medium. Hydrophobic drug is a water insoluble drug. The present invention is applicable equally to both hydrophilic and hydrophobic or lipophilic solid particulate matter.

In certain embodiments, the solid particulate starting material is a pharmaceutically active ingredient. The method disclosed herein can produce bonded microparticulates of a wide variety of pharmaceutical active ingredients, regardless of their solubility, morphology or form (e.g., amorphous or crystalline), particle density, therapeutic category or therapeutic administration route (e.g., oral or injection). Table 1 discloses the classification of exemplary solid particulate matter which can be used to produce BMPX of the present invention and solid particulate material can easily be classified based on its solubility, density and form.

TABLE 1
Classification of solid particulate material - Actives
and Excipients which can be converted into BMPX

| | |
|---|---|
| A. Crystalline Material | B. Amorphous Material |

Actives

| | | | |
|---|---|---|---|
| A.1 Hydrophilic | A.2 Hydrophobic | B.1 Hydrophilic | B.2 Hydrophobic |
| A.1.1 Low Density | A.2.1 Low Density | B.1.1 Low Density | B.2.1 Low Density |
| A.1.2. Medium | A.2.2. Medium | B.1.2. Medium | B.2.2. Medium |
| Density | Density | Density | Density |
| A.1.3. High Density | A.2.3. High Density | B.1.3. High Density | B.2.3. High Density |

Excipient

| | | | |
|---|---|---|---|
| A.1 Hydrophilic | A.2 Hydrophobic | B.1 Hydrophilic | B.2 Hydrophobic |
| A.1.1 Low Density | A.2.1 Low Density | B.1.1 Low Density | B.2.1 Low Density |
| A.1.2. Medium | A.2.2. Medium | B.1.2. Medium | B.2.2. Medium |
| Density | Density | Density | Density |
| A.1.3. High Density | A.2.3. High Density | B.1.3. High Density | B.2.3. High Density |

The present invention can be carried out with any pharmaceutically active ingredient. In certain embodiments, the pharmaceutically active ingredient can be selected from an antidiabetic drug, a therapeutic drug for diabetic complications, a lipid lowering agent, an antihypertensive drug, an anti-obesity drug, an anti-clotting agent, an anticoagulant drug, an opioid for pain management, an analgesic, an anti-inflammatory agent, an antihistamine, a steroid drug, a bronchodilator, an anticholinergic agent, an antibiotic drug, an anti-fungal drug, an antiviral drug, an anti-emetic, a leukotriene receptor antagonist, a sympathomimetic drug, an acetylcholinesterase inhibitor, an immunomodulator, a phosphodiesterase inhibitor, an antidepressant, a serotonin agonist, a serotonin antagonist, an adrenergic agonist, an adrenergic antagonist, an adrenergic neurone blocker, a benzodiazepine, an anticonvulsant, a calcium channel blocker, an antiarrhythmic, a potassium channel modulator, a diuretic, a smoking cessation drug, a bisphosphonate, a dopamine agonist, a nucleic-acid medicine, an antipsychotic, a central nervous system stimulant, an expectorant, an antacid, prebiotics, anti-allergic, a zinc compound used in the treatment of colds. Any other pharmaceutically active agent not discloses herein is also included within the scope of the invention. The present invention also contemplates combination of solid particulate matter e.g. pharmaceutically active drugs. The skilled artisan will know, based on his technical knowledge, which drug combinations are acceptable.

In various embodiments, the pharmaceutically active ingredient is present at high percentage in the composition. High percentage pharmaceutically active ingredient mean that pharmaceutically active ingredient is present in an amount more than 70% by weight based on a total weight of the bonded microparticulates preferably above about 75% by weight, based on a total weight of the bonded microparticulates, more preferably about above 80% by weight, based on a total weight of the bonded micro particulates.

In some embodiments, the solid particulate materials can be present in an amount that is equal to or greater than the amount of bonding agent (e.g. by weight percent, by volume percent, etc.) present in the BMPX composition. In some embodiments, the solid particulate materials can be present in an amount that is between about 1 and about 33 times the amount of bonding agent in the BMPX composition. In another embodiment, the solid particulate materials can be present in an amount that is between about 1 and about 8 times the amount of bonding agent in the BMPX composition. In other embodiments, the amount of solid particulate materials can be at least 2 times the amount of bonding agent in the composition. In yet other embodiments, the amount of solid particulate can be at least 3 times the amount of bonding agent in the BMPX composition. In some embodiments, the amount of the solid particulate materials can be between about 2 times and about 3 times the amount of bonding agent in the BMPX composition.

Different forms of the pharmaceutically active ingredient(s) are also contemplated. The present invention encompasses polymorphs thereof and specific purified enantiomeric forms thereof. In certain embodiments, the active ingredient(s) includes individually optically active enantiomers of the active ingredient(s). Pharmaceutically acceptable salts, such as for example pharmaceutically acceptable addition salts, of the active ingredient(s) are also suitable.

In certain preferred embodiments, the antidiabetic drug can be selected from sulfonylureas, biguanides, thiazolidinediones, meglitinides, dipeptidyl peptidase IV (DPP-IV) inhibitors, α-glucosidase inhibitor, sodium dependent glucose transporter-2 inhibitor (SGLT2) inhibitors, glucagon-like peptide 1 (GLP-1) agonists, insulin and analogues of insulin.

Examples of the “sulfonylureas” include but not limited to glimepiride, glipiride, tolbutamide, chlorpropamide, tolazamide, acetohexamide, glycopyramide, glibenclamide (glyburide), gliclazide, 1-butyl-3-metanilylurea, carbutamide, glibonuride, glipizide, gliquidone, glisoxepid, glybuthiazole, glybuzole, glyhexamide, glymidine, glypinamide, phenbutamide, tolylcyclamide, and pharmaceutically acceptable salts and derivatives thereof.

Examples of the “biguanides” include but not limited to metformin, buformin, phenformin, and pharmaceutically acceptable salts and derivatives thereof.

Examples of the “thiazolidinediones” include but not limited to troglitazone, ciglitazone, pioglitazone, rosiglitazone, darglitazone, englitazone, isaglitazone, ciglitazone, and pharmaceutically acceptable salts and derivatives thereof.

Examples of the “meglitidines” include but not limited to mitiglinide, repaglinide, nateglinide, and pharmaceutically acceptable salts and derivatives thereof.

Examples of the “α-glucosidase inhibitors” include but not limited to voglibose, acarbose, miglitol, emiglitate, and pharmaceutically acceptable salts and derivatives thereof.

Examples of the “DPP-IV inhibitors” include but not limited to sitagliptin, saxagliptin, linagliptin, alogliptin, vildagliptin, gemigliptin, teneligliptin, and pharmaceutically acceptable salts and derivatives thereof.

Examples of the “SGLT2 inhibitors” include but not limited to canagliflozin, dapagliflozin, empagliflozin, ertugliflozin, ipragliflozin, luseogliflozin, remogliflozin, sergliflozin, sotagliflozin, tofogliflozin, and pharmaceutically acceptable salts and derivatives thereof.

Examples of the “GLP-1 agonists” include but not limited to exenatide, liraglutide, semaglutide, lixisenatide, albiglutide and taspoglutide.

Examples of the “insulin and analogues of insulin” include but not limited to Berlinsulin (Berlin-Chemie), Huminsulin (Eli Lilly), Insulin Actrapid (Novo Nordisk), Insuman (Aventis) and the like.

The “therapeutic drug for diabetic complications” can be selected from the group consisting of epalrestat, fidarestat, zenarestat, ruboxistaurin, ranirestat, ALT-946, MCC-257, TAK-428, TAK-128, and pharmaceutically acceptable salts and derivatives thereof.

Examples of the “lipid lowering agent” include but not limited to (i) HMG-COA reductase inhibitors (lovastatin, simvastatin, pravastatin, cerivastatin, fluvastatin, atorvastatin, pitavastatin, and rosuvastatin, and other statins), (ii) sequestrants (cholestyramine, colestipol, and dialkylaminoalkyl derivatives of a cross-linked dextran), (iii) nicotinyl alcohol, nicotinic acid or niacin a salt thereof, (iv) PPARα agonists such as fenofibric acid derivatives (gemfibrozil, clofibrate, fenofibrate and bezafibrate), (v) PPARα/γ dual agonists, such as KRP-297 and muraglitazar, (vi) inhibitors of cholesterol absorption, such as beta-sitosterol and ezetimibe, (vii) acyl CoA: cholesterol acyltransferase inhibitors, such as avasimibe, and (viii) anti-oxidants, such as probucol. Preferably, the lipid lowering agent can be selected from the group consisting of pravastatin, fluvastatin, lovastatin, mevastatin, simvastatin, atorvastatin, pitavastatin, rosuvastatin, cerivastatin, implitapide, fenofibrate, ciprofibrate, gemfibrozil, clofibrate, colestimide (colestilan), colestyramine resin, colestipol, ispaghula, nictotinic acid, acipimox, omega-3 triglycerides, sevelamer hydrochloride, colesevelam hydrochloride, ezetimibe, and pharmaceutically acceptable salts and derivatives thereof.

Examples of the “antihypertensive drug” include but not limited to captopril, fosinopril, enalapril, lisinopril, quinapril, benazepril, fentiapril, ramipril, omapatrilat, fasidotril, irbesartan, losartan, valsartan, candesartan, telmisartan, olmesartan, amlodipine besylate, nifedipine, felodipine, nitrendipine, propranolol, metoprolol, atenolol, carvedilol, betaxolol, prazosin, terazosin, doxazosin, spironolactone, eplerenone, and pharmaceutically acceptable salts and derivatives thereof.

Examples of the “antiobesity drug” include but not limited to fenfluramine, dexfenfluramine, phentermine, sibutramine, orlistat, neuropeptide Y1 or Ys antagonists, CB1 receptor inverse agonists and antagonists, β3 adrenergic receptor agonists, melanocortin-receptor agonists, in particular melanocortin-4 receptor agonists, ghrelin antagonists, bombesin receptor agonists (such as bombesin receptor subtype-3 agonists), and melanin-concentrating hormone (MCH) receptor antagonists. Preferably, the antiobesity drug can be selected from the group consisting of mirabegron, CL-316243, orlistat, cetilistat, sibutramine, mazindol, rimonabant, and pharmaceutically acceptable salts and derivatives thereof.

Examples of the “anti-clotting agent” include but not limited to aspirin, heparin and low molecular weight heparin, epoprostenol, dipyridamole, clopidogrel, alteplase, reteplase, streptokinase, tenecteplase, certoparin, heparin calcium, enoxaparin, dalteparin, danaparoid, fondaparin, lepirudin, bivalirudin, abciximab, eptifibatide, tirofiban, tinzaparin, warfarin, lepirudin, phenindione, acenocoumarol, ticlopidine, cilostazol, sarpogrelate, ozagrel, prasugrel, and pharmaceutically acceptable salts and derivatives thereof.

The “anticoagulant drug” can be selected from the group consisting of warfarin, ximelagatran, aragatroban, low molecule heparin, sofigatran, and pharmaceutically acceptable salts and derivatives thereof.

Examples of the “opioid” include but not limited to buprenorphine, dextromoramide, dextropropoxypene, diamorphine, codeine, dextropropoxyphene, dihydrocodeine, hydromorphone, papaveretum, pholcodeine, loperamide, fentanyl, methadone, morphine, oxycodone, phenazocine, pethidine, tramadol, and pharmaceutically acceptable salts and derivatives thereof.

Examples of the “analgesic” include but not limited to aspirin and other salicylates, paracetamol, clonidine, codeine, coproxamol, ergotamine, gabapentin, pregabalin, sumatriptan, and non-steroidal anti-inflammatory drugs (NSAIDs) including celecoxib, etodolac, etoricoxib, meloxicam, and pharmaceutically acceptable salts and derivatives thereof.

Examples of the “anti-inflammatory agent” include but not limited to piroxicam, nedocromil, benzydamine, diclofenac, ketoprofen, ibuprofen, heparinoid, cromoglycate, fasafungine, iodoxamide, p38 MAP kinase inhibitors, and pharmaceutically acceptable salts and derivatives thereof.

Examples of the “antihistamine” include but not limited to azelastine, chlorpheniramine, astemizole, cetirizine, cinnarizine, desloratadine, loratadine, hydroxyzine, diphenhydramine, fexofenadine, ketotifen, promethazine, trimeprazine, terfenadine, and pharmaceutically acceptable salts and derivatives thereof.

Examples of the “steroid drug” include but not limited to alcometasone, beclomethasone, beclomethasone dipropionate, betamethasone, budesonide, ciclesonide, clobetasol, deflazacort, diflucortolone, desoxymethasone, dexamethasone, fludrocortisone, flunisolide, fluocinolone, fluometholone, fluticasone, fluticasone proprionate, hydrocortisone, triamcinolone, nandrolone decanoate, neomycin sulphate, rimexolone, methylprednisolone, prednisolone, and pharmaceutically acceptable salts and derivatives thereof.

Examples of the “bronchodilator” include but not limited to B2-agonists including salbutamol, formoterol, salmeterol, fenoterol, bambuterol, bitolterol, sibenadet, metaproterenol, epinephrine, isoproterenol, pirbuterol, procaterol, terbutaline and isoetharine antimuscarinics including ipratropium and tiotropium, and xanthines including aminophylline and theophylline, and pharmaceutically acceptable salts and derivatives thereof.

Examples of the “anticholinergic agent” include but not limited to atropine, benzatropine, biperiden, cyclopentolate, oxybutinin, orphenadine, glycopyrronium, glycopyrrolate, procyclidine, propantheline, propiverine, tiotropium, trihexyphenidyl, tropicamide, trospium, ipratropium, oxitroprium, and pharmaceutically acceptable salts and derivatives thereof.

Examples of the “antibiotic drug” include but not limited to cephalosporin antibiotics, β-lactam antibiotics, metronidazole, sulphadiazine, triclosan, neomycin, amoxicillin, amphotericin, clindamycin, aclarubicin, dactinomycin, nystatin, mupirocin, chlorhexidine, and pharmaceutically acceptable salts and derivatives thereof.

Examples of cephalosporin antibiotics include cefatrizine, cephaloridine, cephalothin, cefazolin, cephalexin, cephacetrile, cephapirin, cephamandolenafate, cephradine, 4-hydroxy cephalexin, cephaloglycin, cefoperazone, cefsulodin, ceftazidime, cefuroxime, cefinetazole, cefotaxime, ceftriaxone, ceftazidime, ceftabiprole, ceftarolinefosamil, and other known cephalosporins, all of which may be used in the form of salts or prodrugs thereof.

Examples of β-lactam antibiotics include aztreonam and carbapenems like imipenem and meropenem.

Examples of the “anti-fungal drug” include but not limited to caspofungin, voriconazole, polyene antibiotics including amphotericin, and nystatin, imidazoles and triazoles including clotrimazole, econazole nitrate, fluconazole, ketoconazole, itraconazole, terbinafine and miconazole, and pharmaceutically acceptable salts and derivatives thereof.

Examples of the “antiviral drug” include but not limited to oseltamivir, zanamivir, amantadine, inosine pranobex and palivizumab, DNA polymerase inhibitors including aciclovir, adefovir and valaciclovir, nucleoside analogues including famiciclovir, penciclovir and idoxuridine and interferons, and pharmaceutically acceptable salts and derivatives thereof.

Examples of the “anti-emetics” include but not limited to bestahistine, dolasetron, nabilone, prochlorperazine, ondansetron, trifluoperazine, tropisetron, domperidone, hyoscine, cinnarizine, metoclopramide, cyclizine, dimenhydrinate, promethazine, and pharmaceutically acceptable salts and derivatives thereof.

The leukotriene receptor antagonist can be selected from montelukast and zafirlukast.

Examples of the “sympathomimetic drug” include but not limited to adrenaline, noradrenaline, dexamfetamine, dipirefin, dobutamine, dopexamine, phenylephrine, isoprenaline, dopamine, pseudoephedrine, tramazoline, xylometazoline, and pharmaceutically acceptable salts and derivatives thereof.

Examples of the “acetylcholinesterase inhibitor” include but not limited to donepezil, galantamine, rivastigmine, and pharmaceutically acceptable salts and derivatives thereof.

Examples of the “immunomodulator” include but not limited to interferon (e.g. interferon beta-la and interferon beta-Ib) and glatiramer.

Examples of the “phosphodiesterase inhibitor” include but not limited to non-specific phosphodiesterase inhibitors including theophylline, theobromine, IBMX, pentoxifylline and papaverine; phosphodiesterase type 3 inhibitors including bipyridines such as milrinone, amrinone and olprinone; imidazolones such as piroximone and enoximone; imidazolines such as imazodan and 5-methyl-imazodan; imidazo-quinoxalines; and dihydropyridazinones such as indolidan and LY181512 (5-(6-oxo-1,4,5,6-tetrahydro-pyridazin-3-yl)-1,3-dihydro-indol-2-one); dihydroquinolinone compounds such as cilostamide, cilostazol, and vesnarinone; phosphodiesterase type 4 inhibitors such as cilomilast, etazolate, rolipram, roflumilast and zardaverine, and including quinazolinediones such as nitraquazone and nitraquazone analogs; xanthine derivatives such as denbufylline and arofylline; tetrahydropyrimidones such as atizoram; and oxime carbamates such as filaminast; and phosphodiesterase type 5 inhibitors including sildenafil, zaprinast, vardenafil, tadalafil, dipyridamole, and the compounds described in WO 01/19802, particularly(S)-2-(2-hydroxymethyl-1-pyrrolidinyl)-4-(3-chloro-4-methoxy-benzylamino)-5-[N-(2-pyrimidinylmethyl) carbamoyl]pyrimidine, 2-(5,6,7,8-tetrahydro-1,7-naphthyridin-7-yl)-4-(3-chloro-4-methoxybenzylamino)-5-[N-(2-morpholinoethyl) carbamoyl]-pyrimidine, and(S)-2-(2-hydroxymethyl-1-pyrrolidinyl)-4-(3-chloro-4-methoxy-benzylamino)-5-[N-(1,3,5-trimethyl-4-pyrazolyl) carbamoyl]-pyrimidine), and pharmaceutically acceptable salts and derivatives thereof.

Examples of the “antidepressant” include but not limited to tricyclic and tetracyclic antidepressants including amineptine, amitriptyline, amoxapine, butriptyline, cianopramine, clomipramine, dosulepin, doxepin, trimipramine, clomipramine, lofepramine, nortriptyline, tricyclic and tetracyclic amitryptiline, amoxapine, butriptyline, clomipramine, demexiptiline, desipramine, dibenzepin, dimetacrine, dothiepin, doxepin, imipramine, iprindole, levoprotiline, lofepramine, maprotiline, melitracen, metapramine, mianserin, mirtazapine, nortryptiline, opipramol, propizepine, protriptyline, quinupramine, setiptiline, tianeptine and trimipramine; selective serotonin and noradrenaline reuptake inhibitors (SNRIs) including clovoxamine, duloxetine, milnacipran and venlafaxine; selective serotonin reuptake inhibitors (SSRIs) including citalopram, escitalopram, femoxetine, fluoxetine, fluvoxamine, ifoxetine, milnacipran, nomifensine, oxaprotiline, paroxetine, sertraline, sibutramine, venlafaxine, viqualine and zimeldine; selective noradrenaline reuptake inhibitors (NARIs) including demexiptiline, desipramine, oxaprotiline and reboxetine; noradrenaline and selective serotonin reuptake inhibitors (NASSAs) including mirtazapine; monoamine oxidase inhibitors (MAOIs) including amiflamine, brofaromine, clorgyline, α-ethyltryptamine, etoperidone, iproclozide, iproniazid, isocarboxazid, mebanazine, medifoxamine, moclobemide, nialamide, pargyline, phenelzine, pheniprazine, pirlindole, procarbazine, rasagiline, safrazine, selegiline, toloxatone and tranylcypromine; muscarinic antagonists including benactyzine and dibenzepin; azaspirones including buspirone, gepirone, ipsapirone, tandospirone and tiaspirone; and other antidepressants including amesergide, amineptine, benactyzine, bupropion, carbamazepine, fezolamine, flupentixol, levoprotiline, maprotiline, medifoxamine, methylphenidate, minaprine, nefazodone, nomifensine, oxaflozane, oxitriptan, rolipram, sibutramine, teniloxazine, tianeptine, tofenacin, trazadone, tryptophan, viloxazine, and lithium salts, and pharmaceutically acceptable salts and derivatives thereof.

Examples of the “serotonin agonist” include but not limited to 2-methyl serotonin, buspirone, ipsaperone, tiaspirone, gepirone, lysergic acid diethylamide, ergot alkaloids, 8-hydroxy-(2-N,N-dipropylamino)-tetraline, 1-(4-bromo-2,5-dimethoxyphenyl)-2-aminopropane, cisapride, sumatriptan, m-chlorophenylpiperazine, trazodone, zacopride, mezacopride, and pharmaceutically acceptable salts and derivatives thereof.

Examples of the “serotonin antagonist” include but not limited to ondansetron, granisetron, metoclopramide, tropisetron, dolasetron, trimethobenzamide, methysergide, risperidone, ketanserin, ritanserin, clozapine, amitryptiline, R (+)-α-(2,3-dimethoxyphenyl)-1-[2-(4-fluorophenyl)ethyl]-4-piperidine-methanol, azatadine, cyproheptadine, fenclonine, dexfenfluramine, fenfluramine, chlorpromazine, mianserin, and pharmaceutically acceptable salts and derivatives thereof.

Examples of the “adrenergic agonist” include but not limited to methoxamine, methpentermine, metaraminol, mitodrine, clonidine, apraclonidine, guanfacine, guanabenz, methyldopa, amphetamine, methamphetamine, epinephrine, norepinephrine, ethylnorepinephrine, phenylephrine, ephedrine, pseudo-ephedrine, methylphenidate, pemoline, naphazoline, tetrahydrozoline, oxymetazoline, xylometazoline, phenylpropanolamine, phenylethylamine, dopamine, dobutamine, colterol, isoproterenol, isotharine, metaproterenol, terbutaline, metaraminol, tyramine, hydroxyamphetamine, ritodrine, prenalterol, albuterol, isoetharine, pirbuterol, bitolterol, fenoterol, formoterol, procaterol, salmeterol, mephenterine, propylhexedrine, and pharmaceutically acceptable salts and derivatives thereof.

Examples of the “adrenergic antagonist” include but not limited to phenoxybenzamine, phentolamine, tolazoline, prazosin, terazosin, doxazosin, trimazosin, yohimbine, ergot alkaloids, labetalol, ketanserin, urapidil, alfuzosin, bunazosin, tamsulosin, chlorpromazine, haloperidol, phenothiazines, butyrophenones, propranolol, nadolol, timolol, pindolol, metoprolol, atenolol, esmolol, acebutolol, bopindolol, carteolol, oxprenolol, penbutolol, carvedilol, medroxalol, naftopidil, bucindolol, levobunolol, metipranolol, bisoprolol, nebivolol, betaxolol, carteolol, celiprolol, sotalol, propafenone, indoramin, and pharmaceutically acceptable salts and derivatives thereof.

Examples of the “adrenergic neurone blocker” include but not limited to bethanidine, debrisoquine, guabenxan, guanadrel, guanazodine, guanethidine, guanoclor, guanoxan, and pharmaceutically acceptable salts and derivatives thereof.

Examples of the “benzodiazepine” include but not limited to alprazolam, bromazepam, brotizolam, chlordiazepoxide, clobazam, clonazepam, clorazepate, demoxepam, diazepam, estazolam, flunitrazepam, flurazepam, halazepam, ketazolam, loprazolam, lorazepam, lormetazepam, medazepam, midazolam, nitrazepam, nordazepam, oxazepam, prazepam, quazepam, temazepam, triazolam, and pharmaceutically acceptable salts and derivatives thereof.

Examples of the “anticonvulsant” include but not limited to sodium valproate, carbamazepine, oxcarbazepine, phenytoin, fosphenytoin, diazepam, lorazepam, clonazepam, clobazam, primidone, lamotrigine, levetiracetam, topiramate, gabapentin, pregabalin, vigabatrin, tiagabine, acetazolamide, ethosuximide, piracetam, and pharmaceutically acceptable salts and derivatives thereof.

Examples of the “calcium channel blocker” include but not limited to amlodipine, bepridil, diltiazem, felodipine, flunarizine, isradipine, lacidipine, lercanidipine, nicardipine, nifedipine, nimodipine, verapamil, and pharmaceutically acceptable salts and derivatives thereof.

Examples of the “antiarrhythmic” include but not limited to adenosine, propafenone, amidodarone, flecainide acetate, quinidine, lidocaine, mexiletine, procainamide, disopyramide, and pharmaceutically acceptable salts and derivatives thereof.

Examples of the “potassium channel modulator” include but not limited to nicorandil, cromakalim, diazoxide, glibenclamide, levcromakalim, minoxidil, pinacidil, and pharmaceutically acceptable salts and derivatives thereof.

Examples of the “diuretic” include but not limited to bumetanide, furosemide, torasemide, spironolactone, amiloride, bendroflumethiazide, chlortalidone, metolazone, indapamide, cyclopenthiazide, and pharmaceutically acceptable salts and derivatives thereof. In an embodiment, the smoking cessation drug can be selected from nicotine and bupropion.

Examples of the “bisphosphonate” include but not limited to alendronate sodium, sodium clodronate, etidronate disodium, ibandronic acid, pamidronate disodium, isedronate sodium, tiludronic acid, zoledronic acid, and pharmaceutically acceptable salts and derivatives thereof.

Examples of the “dopamine agonist” include but not limited to amantadine, bromocriptine, pergolide, cabergoline, lisuride, ropinerole, pramipexole, apomorphine, and pharmaceutically acceptable salts and derivatives thereof.

Examples of the “nucleic-acid medicine” include but not limited to oligonucleotides, decoy nucleotides, antisense nucleotides and other gene-based medicine molecules.

Examples of the “antipsychotic” include but not limited to dopamine antagonists including chlorpromazine, prochlorperazine, fluphenazine, trifluoperazine and thioridazine; phenothiazines including aliphatic compounds, piperidines and piperazines; thioxanthenes, butyrophenones and substituted benzamides; atypical antipsychotics including clozapine, risperidone, olanzapine, quetiapine, ziprasidone, zotepine, amisulpride and aripiprazole, and pharmaceutically acceptable salts and derivatives thereof.

Examples of the “central nervous system stimulants” include but not limited toatomoxetine, reboxetine, yohimbine, caffeine, phenmetrazine, phendimetrazine, pemoline, fencamfamine, fenethylline, pipradol, deanol, methylphenidate, methylphenidate hydrochloride, dexmethylphenidate, amphetamine, dextroamphetamine sulfate, methamphetamine, lisdexamfetamine and benzphetamine, and pharmaceutically acceptable salts and derivatives thereof.

Examples of the “antacids” include but not limited to sodium bicarbonate, calcium carbonate, aluminum hydroxide and magnesium hydroxide.

Examples of “expectorants” include but not limited to guaifenesin, potassium guaiacolsulfonate, ipecac, potassium iodide, and tenpin hydrate.

Examples of “zinc compound” include organic and inorganic salts of zinc. Non-limiting examples of the inorganic salts of zinc include zinc bromide, zinc chloride, zinc iodine, zinc fluoride, zinc ammonium sulfate, zinc chromate, zinc fluorosilicate, zinc dithionate, zinc sulfate, zinc nitrate, zinc phosphate, zinc containing fluorozirconate, zinc oxide, and mixtures thereof. Non-limiting examples of the organic salts of zinc include zinc citrate, zinc acetate, zinc gluconate, zinc aspartate, zinc ascorbate, zinc orotate, zinc divalent amino acid zinc salts, zinc succinate, zinc tartrate, zinc glycerophosphate, zinc salicylate, zinc formate, and mixtures thereof.

In certain embodiments, the solid particulate material is a bioactive agent. Non-limiting examples of bioactive agent include antibodies, antibody fragments, proteins, polypeptides, peptides, fusion proteins (e.g., Ig fusion proteins or Fc fusion proteins), multivalent binding proteins (e.g., DVD Ig), antibody-drug conjugates, vaccines, nucleic acids, sugars, recombinant forms thereof, engineered forms thereof, and combinations thereof.

In certain embodiments, the solid particulate material is a dietary supplement. Non-limiting examples of dietary active ingredient include vitamins, minerals, herbs, amino acids, fatty acids, probiotics, dietary fiber materials, calcium supplements, carbohydrates, glycoconjugates, substances such as enzymes and metabolites to supplement diet by increasing total dietary intake, and combinations thereof.

In various embodiments, the dietary supplement is present at high percentage in the composition. High percentage dietary supplement means present in an amount more than 70% by weight based on a total weight of the bonded microparticulates preferably above about 75% by weight, based on a total weight of the bonded microparticulates, more preferably about above 80% by weight, based on a total weight of the bonded micro particulates.

Non-limiting examples of vitamins include vitamin A, vitamin D, vitamin E, vitamin K, thiamine, riboflavin, pyridoxine, vitamin B12, niacin, folic acid, pantothenic acid, biotin, vitamin C, choline, chromium, carnitine, inositol, salts and derivatives thereof, and combinations thereof.

Examples of the dietary fiber materials include high fiber dextrin such as Fibersol-2®., Oliogofructose saccharides, Acacia, carrageenan, wheat fiber, pea fiber, and vegetable fibers, Animal fibers etc

Examples of “calcium and other mineral supplements” include but not limited to calcium carbonate, calcium citrate, calcium hydroxide, calcium phosphate, calcium chlorophosphate, and combinations thereof.

In certain embodiments, the solid particulate material is a non-active ingredient useful as pharmaceutical excipient in the preparation of pharmaceutical dosage forms. The method disclosed herein is capable of agglomerating any pharmaceutical excipient that is in a powder or particulate form into bonded microparticulates of a desired size and character, regardless of the excipient's solubility in aqueous or nonaqueous medium, morphology (e.g., amorphous or crystalline), particle density, particle size, hydrophilicity, hydrophobicity The excipient may be any substance used as a carrier, diluent, adjuvant and/or vehicle for delivery of a therapeutic agent to a patient, or added to a pharmaceutical composition to improve its handling or storage properties or to permit or facilitate formation of a compound or pharmaceutical composition into a unit dosage form for administration. Non-limiting examples of such excipient include mannitol, xylitol, sugar powder, sorbitol, glucose, sucrose, dextrose, lactose, microcrystalline cellulose, calcium phosphate, dicalcium phosphate, fructose, calcium sulfate, calcium carbonate, magnesium salts, sodium bicarbonate, and the like, or mixtures thereof. In various embodiments, a non-active ingredient useful as pharmaceutical excipient is present at high percentage in the composition. High percentage non-active ingredient useful as pharmaceutical excipient is present in an amount more than 70% by weight based on a total weight of the bonded microparticulates preferably above about 75% by weight, based on a total weight of the bonded microparticulates, more preferably about above 80% by weight, based on a total weight of the bonded micro particulates.

In other embodiments the solid particulate material can be oral care materials including but not limited to one or more of fluoride ion sources, anticalculus or anti-tartar agents, antimicrobial agents, anti-dry mouth agents, buffers, pH modifiers, like Sodium bicarbonate or sodium carbonates and or potassium bicarbonate or related salts abrasives such a silica, alkali metal bicarbonate salts, thickening materials, humectants, water, surfactants, titanium dioxide, flavorants, sweetening agents, coolants and other sensates, xylitol, and coloring agents. More preferably the oral care materials include Xylitol, Sorbitol, Maltitol, Erythritol, Alluolse, Mannnitol or polyols or zinc salts which are non-carcinogenic and tooth friendly. Other Sugar based materials can also be used as oral care material. The oral care agent is present at high percentage in the composition. High percentage oral care agent is present in an amount more than 70% by weight based on a total weight of the bonded microparticulates preferably above about 75% by weight, based on a total weight of the bonded microparticulates, more preferably about above 80% by weight, based on a total weight of the bonded micro particulates.

In some embodiments, the solid particulate starting material is a food ingredient. Non-limiting examples of food ingredients in accordance with the present disclosure include food additives, food flavoring excipients, and ingredients used in dry beverage mix. The food ingredient is present at high percentage in the composition. High percentage food ingredient pient is present in an amount more than 70% by weight based on a total weight of the bonded microparticulates preferably above about 75% by weight, based on a total weight of the bonded microparticulates, more preferably about above 80% by weight, based on a total weight of the bonded micro particulates.

In some embodiments, the solid particulate starting material is an agrochemical.

In some embodiments, the solid particulate starting material is an animal food.

As indicated above, the bonding agent is a solid or semi-solid or viscous or liquid material at room temperature, which softens at a temperature not exceeding the melting point (or melting range) of the solid particulate material when heated at a temperature between room temperature and the melting point of the bonding agent. Typically, the bonding agent softens at a temperature between 30° C. to 100° C. Preferably, the temperature at which bonding agent softens is from about 40° C. to about 70° C. Preferably the bonding agent softens when heated around a temperature of 60° C. depending upon the melting point of bonding agent.

In various embodiments, the bonding agent can be a polymer, an emulsifier, a fat or a combination thereof. In another embodiment, the present invention includes one or more than one bonding agent. The present invention contemplates use of low percentage of bonding agent. Low percentage of bonding agent mean that the bonding agent is used in an amount up to about 50% by weight, preferably up to about 30% by weight, and more preferably up to 20% by weight and most preferably up to about 18% by weight, based on a total weight of the bonded microparticulates. Obviously, if the bonded microparticulate is to be used as a pharmaceutical, the bonding agent must be non-toxic and not affect the pharmaceutical activity of the pharmaceutical active ingredient (solid particulate starting material).

In some embodiments, the bonding agent can be a polymer. Types of polymers include, but are not limited to, water-soluble, water-swellable, water insoluble polymers and combinations thereof. Examples of polymers include, but are not limited to: homopolymers and copolymers of N-vinyl lactams, e.g., homopolymers and copolymers of N-vinyl pyrrolidone (e.g., polyvinylpyrrolidone), copolymers of N-vinyl pyrrolidone and vinyl acetate or vinyl propionate; cellulose esters and cellulose ethers (e.g., methylcellulose and ethylcellulose) hydroxyalkylcelluloses (e.g., hydroxypropylcellulose), hydroxyalkylalkylcelluloses (e.g., hydroxypropylmethylcellulose), cellulose phthalates (e.g., cellulose acetate phthalate and hydroxylpropylmethylcellulose phthalate) and cellulose succinates (e.g., hydroxypropylmethylcellulose succinate or hydroxypropylmethylcellulose acetate succinate); high molecular polyalkylene oxides such as polyethylene oxide and polypropylene oxide and copolymers of ethylene oxide and propylene oxide; polyacrylates and polymethacrylates (e.g., methacrylic acid/ethyl acrylate copolymers, methacrylic acid/methyl methacrylate copolymers, butyl methacrylate/2-dimethylaminoethyl methacrylate copolymers, poly(hydroxyalkyl acrylates), poly(hydroxyalkyl methacrylates)); polyacrylamides; vinyl acetate polymers such as copolymers of vinyl acetate and crotonic acid, partially hydrolyzed polyvinyl acetate; and oligo- and polysaccharides, such as carrageenans, galactomannans and xanthan gum, Polyethylene glycol 2000, Polyethylene glycol 3000, Polyethylene glycol 6000, Polyethylene glycol 8000, Polyethylene glycol 10000, Polyethylene glycol 20000 and polyethylene glycol palmitostearate (e.g., Stearate 6000 WL 1644®), Polyvinyl alcohol and acetate, Ethyl Vinayl acetate. The most preferred polymer which was used to prepare bonded microparticulates is the polyethylene glycol.

In some embodiments, the bonding agent can be an emulsifier. Examples of emulsifier are polyethoxylated fatty acids such as PEG-8 laurate, PEG-8 oleate, PEG-8 stearate, PEG-9 oleate, PEG-10 laurate, PEG-10 oleate, PEG-12 laurate, PEG-12 oleate, PEG-15 oleate, PEG-20 laurate and PEG-20 oleate; PEG-fatty acid diesters such as PEG-20 dilaurate, PEG-dioleate, PEG-20 distearate, PEG-32 dilaurate and PEG-32 dioleate; PEG-fatty acid mono- and di-ester mixtures; polyethylene glycol glycerol fatty acid esters such as PEG-20 glyceryl laurate, PEG-30 glyceryl laurate, PEG-40 glyceryl laurate, PEG-20 glyceryl oleate, and PEG-glyceryl oleate; alcohol-oil transesterification products such as PEG-35 castor oil (Incrocas-35), PEG-40 hydrogenated castor oil (Cremophor® RH40), polyoxyl 35 castor oil (Cremophor EL), PEG-25 trioleate (TAGAT® TO), PEG-60 corn glycerides (Crovol M70), PEG-60 almond oil (Crovol A70), PEG-40 palm kernel oil (Crovol PK70), PEG-50 castor oil (Emalex C-50), PEG-50 hydrogenated castor oil (Emalex HC-50), PEG-8 caprylic/capric glycerides (Labrasol®), and PEG-6 caprylic/capric glycerides (Softigen® 767); transesterification products of oils and alcohols; polyglycerized fatty acids such as polyglyceryl oleate (Plurol® Oleique), polyglyceryl-2 dioleate (Nikkol DGDO), and polyglyceryl-10 trioleate. Preferred emulsifier includes polyglyceryl-10 laurate (Nikkol Decaglyn 1-L), polyglyceryl-10 oleate (Nikkol Decaglyn 1-0), and polyglyceryl-10 mono, dioleate (Caprol® PEG 860); propylene glycol fatty acid esters such as propylene glycol monolaurate (Lauroglycol FCC), propylene glycol ricinoleate (Propymuls), propylene glycol monooleate (Myverol® P-06), propylene glycol dicaprylate/dicaprate (Captex® 200), and propylene glycol dioctanoate (Captex 800); mixtures of propylene glycol esters and glycerol esters such as a mixture of oleic acid esters of propylene glycol and glycerol (Arlacel 186); mono- and diglycerides such as glyceryl monooleate (Peceol), glyceryl ricinoleate, glyceryl laurate, glyceryl dilaurate (Capmul® GDL), glyceryl dioleate (Capmul GDO), glyceryl mono/dioleate (Capmul GMO-K), glyceryl caprylate/caprate (Capmul MCM), caprylic acid mono/diglycerides (Imwitor® 988), and mono- and diacetylated monoglycerides (Myvacet® 9-45); sterol and sterol derivatives such as PEG-24 cholesterol ether (Solulan® C-24); polyethylene glycol sorbitan fatty acid esters such as PEG-20 sorbitan monolaurate (Tween® 20), PEG-20 sorbitan monopalmitate (Tween 40), PEG-20 sorbitan monostearate (Tween 60), and PEG-20 sorbitan monooleate (polysorbate 80 or Tween 80); polyethylene glycol alkyl ethers such as PEG-3 oleyl ether (Volpo 3) and PEG-4 lauryl ether (Brij 30); sugar esters such as sucrose monopalmitate and sucrose monolaurate; polyethylene glycol alkyl phenols; polyoxyethylene-polyoxypropylene block copolymers such as Synperonic® PE series (ICI); Pluronic® series (BASF), Emkalyx, Lutrol (BASF), Supronic, Monolan, Pluracare®, and Plurodac; sorbitan fatty acid esters such as sorbitan monolaurate (Arlacel® 20), sorbitan monopalmitate (Span-40), sorbitan monooleate (Span-80), sorbitan monostearate, and sorbitan tristearate; lower alcohol fatty acid esters such as hydrophobic surfactants include ethyl oleate (Crodamol EO), isopropyl myristate (Crodamol IPM), and isopropyl palmitate (Crodamol IPP), PEG-400 succinate, PEG 3350, tocopherol polyethyleneglycol (200-8000 MW) succinate, tocopherol polyethylene glycol 400 succinate, tocopherol polyethyleneglycol 1000 succinate (Vitamin E-TPGS, Eastman Chemical Co.), glycerol monolinoleate (Maisine®), propylene glycol monocaprylate (Capryol® 90); caprylocaproyl macrogol-8 glycerides (Labrosol®), glycerol dibehenate (Compritol® 888), glycerol distearate (Precirol®), lauroyl macrogol-32 glycerides (Gelucire® 44/14), and stearoyl macrogol-32 glycerides (Gelucire 50/13). The most preferred emulsifier used as bonding material is monoglycerides made from totally hydrogenated palm oil.

In some embodiments, the bonding agent can be a fat. The fat which can be used as the bonding agent includes fats, triglycerides, hydrogenated natural oil or non-hydrogenated fat-fractionated Palm oils or others similar. In some embodiments, the hydrogenated natural oil is selected from the group consisting of hydrogenated canola oil, hydrogenated rapeseed oil, hydrogenated coconut oil, hydrogenated corn oil, hydrogenated cottonseed oil, hydrogenated olive oil, hydrogenated palm oil, hydrogenated peanut oil, hydrogenated safflower oil, hydrogenated sesame oil, hydrogenated soybean oil, hydrogenated sunflower oil, hydrogenated linseed oil, hydrogenated palm kernel oil, hydrogenated tung oil, hydrogenated jatropha oil, hydrogenated mustard oil, hydrogenated camelina oil, hydrogenated pennycress oil, hydrogenated castor oil, hydrogenated derivatives of these oils, and mixtures thereof. The most preferred fat used as bonding agent is hydrogenated soyabean oil.

In one of the embodiments the bonded microparticulate may comprise of one or combination of bonding agents.

In one embodiment, the bonded microparticulates of the present invention is prepared by a) softening bonding agent at a temperature between room temperature and the melting point of the bonding agent to obtain a softened bonding agent; b) contacting the solid particulate material with the softened bonding agent to form a mixture comprising the solid particulate material and the bonding agent; c) cooling the mixture to a predetermined temperature to produce bonded microparticulate

In one embodiment, the bonded microparticulates of the present invention is prepared by a) softening a mixture of bonding agent at a temperature between room temperature and the melting point of the bonding agent to obtain a softened bonding agent; b) contacting the solid particulate material with the softened bonding agent to form a mixture comprising the solid particulate material and the bonding agent; c) cooling the mixture to a predetermined temperature to produce bonded microparticulate.

In one embodiment, the bonded microparticulates of the present invention is prepared by a) softening a mixture of bonding agent at a temperature between room temperature and the melting point of the bonding agent to obtain a softened bonding agent; b) adding up to 3% of water in the softened bonding agent c) contacting the solid particulate material with the softened bonding agent of step b) to form a mixture comprising the solid particulate material and the bonding agent; d) cooling the mixture to a predetermined temperature to produce bonded microparticulate.

In one embodiment, the bonded microparticulates of the present invention is prepared by a) softening a mixture of polymer and emulsifier at a temperature between room temperature and the melting point of the bonding agent to obtain a softened bonding agent; b) contacting the solid particulate material with the softened bonding agent to form a mixture comprising the solid particulate material and the bonding agent; c) cooling the mixture to a predetermined temperature to produce bonded microparticulate. The bonding agent, polymer and emulsifier, can be softened separately and then mixed together or mixed before softening them.

In one embodiment, the bonded microparticulates of the present invention is prepared by a) softening a mixture of polymer and fat at a temperature between room temperature and the melting point of the bonding agent to obtain softened bonding agent; b) contacting the solid particulate material with the softened bonding agent to form a mixture comprising the solid particulate material and the bonding agent; c) cooling the mixture to a predetermined temperature to produce bonded microparticulate. The bonding agent, polymer and fat, can be softened separately and then mixed together or mixed before softening them.

In one embodiment, the bonded microparticulates of the present invention is prepared by a) softening a mixture of fat and emulsifier at a temperature between room temperature and the melting point of the bonding agent to obtain softened bonding agent; b) contacting the solid particulate material with the softened bonding agent to form a mixture comprising the solid particulate material and the bonding agent; c) cooling the mixture to a predetermined temperature to produce bonded microparticulate. The bonding agent, fat and emulsifier, can be softened separately and then mixed together or mixed before softening them.

In one embodiment, the bonded microparticulates of the present invention is prepared by a) softening emulsifier at a temperature between room temperature and the melting point of the bonding agent to obtain softened bonding agent; b) mixing water (may be warmed) with softened emulsifier c) contacting the solid particulate material with the softened bonding agent of step b) to form a mixture comprising the solid particulate material and the bonding agent; d) cooling the mixture to a predetermined temperature to produce bonded microparticulate.

In various embodiments, the bonded microparticulates produced according to the present disclosure may be formulated into dosage forms, such as oral dosage forms (e.g., tablets, capsules, pills, lozenges, caplets, suspensions, rapid-melt tablets, chew tablets, rapid-melt beads), dry injectable compositions or topical skin care compositions, in a conventional manner, by adding one or more pharmaceutically acceptable excipients. Non-limiting examples of such excipients include diluents, drug release-controlling excipients, binders, disintegrants, lubricants, glidants, surfactants, stabilizers, preservatives, alkalizing agents, anti-adherents, sweeteners, flavoring agents, coloring agents, or mixtures thereof.

In some embodiments, bonded microparticulates prepared according to the method of the present disclosure are formulated into tablets by adding one or more pharmaceutically acceptable excipients. In preferred embodiments, the diluent is present in an amount of from about 1% to about 50% by weight of the tablet, the disintegrant is present in an amount of from about 1% to about 10% by weight of the tablet, the lubricant is present in an amount of from about 0.1% to about 5% by weight of the tablet, the glidant is present in an amount of from about 0.1% to about 3% by weight of the tablet, and the surfactant is present in an amount of from about 0.1% to about 5% by weight of the tablet

Compared to prior art techniques, such as wet granulation, dry granulation, spray drying, melt granulation extrusion speronization, use of lipid excipients, self-emulsifying drug delivery systems, solid dispersion and the like preparing composition, the composition of the present invention provides a robust, unique, cost effective and less time-consuming process. The prior art techniques require setting up of huge equipment's such as extruder-spheronizer, fluid bed dryer, spray dryer, etc which have enormous cost and require lot of space for the preparing the finished dosage form. The invention of the present invention is carried out by softening the bonding material and then contacting the solid particulate material with softened bonding material and then subsequent cooling to get bonded microparticulates. The present invention avoids all the processing steps such as mixing and premixing of ingredients, preparation of granulating agent, spray or fluidized bed drying, extrusion to prepare rod shaped particle, spheronization in which the extrudate is transformed from rod-shaped pellets into spherical particles, drying or milling before blending with other inactive material to prepare the final dosage form or composition.

In addition, due to the multistep process of the prior art techniques, the loss of solid particulate material takes place which further increase the cost of goods. The present invention can be contemplated to be completed in minimum steps of softening the bonding agent, contacting the solid particulate material with bonding agent and then cooling the mixture to a predetermined temperature to produce bonded microparticulate. The bonded microparticulate may then be directly compressed or mixed with inactive excipients to prepare tablet dosage form or filled in capsules. Further the prior art methods are not suitable for all types of solid particulate material irrespective of their forms such as crystalline or amorphous, differing densities and differing solubilities. Thus, depending upon the properties of solid particulate material, the method of granulation or granulation techniques needs to be customized. For eq solid dispersion is used for crystalline material, use of lipid excipient is used for poorly soluble drugs, wet granulation for solid particulate material with compressibility issues. The present invention contemplates to prepare the bonded microparticulates and further their composition using solid particulate material whether crystalline or amorphous, low, medium or high-density material or hydrophilic or hydrophobic materials.

Another embodiment discloses, a tablet formulation comprising bonded microparticulates comprising of solid particulate material and bonding agent and excipients. The excipients can be selected from diluents, drug release-controlling excipients, binders, disintegrants, lubricants, glidants, surfactants, stabilizers, preservatives, alkalizing agents, anti-adherents, sweeteners, flavoring agents, coloring agents, or mixtures thereof more preferably, diluent, disintegrant and glidant

As indicated above, dosage forms may comprise a diluent. Examples of diluents include lactose, mannitol, sodium starch glycolate, corn starch, talc, sucrose, dextrose, glucose, lactose, xylitol, fructose, sorbitol, calcium phosphate, calcium sulfate, calcium carbonate, and the like, or mixtures thereof. Microcrystalline cellulose may also be used as a diluent and may be any suitable form of microcrystalline cellulose as is known and used in the tabletting art. The diluent may be used in an amount ranging from about 0% to about 90% by weight based on a total weight of the dosage form, preferably in an amount of from about 1% to about 50% by weight based on a total weight of the dosage form.

As indicated above, dosage forms may comprise a disintegrant. A disintegrant is a substance or a mixture of substances added to facilitate breakup or disintegrate after administration. The disintegrant may be any pharmaceutically acceptable disintegrant available in the tabletting art, including alginic acid, carboxymethylcellulose calcium, carboxymethylcellulose sodium, colloidal silicon dioxide, croscarmellose sodium, crospovidone, sodium starch glycolate, guar gum, magnesium aluminum silicate, methylcellulose, microcrystalline cellulose, polyacrilin potassium, powdered cellulose, pregelatinized starch, sodium alginate, starch, and the like, or mixtures thereof. The pharmaceutical dosage forms may comprise disintegrant in an amount ranging from about 0% to about 15% by weight based on a total weight of the dosage forms, preferably the disintegrant is present in an amount of from about 1% to about 10% by weight based on a total weight of the dosage forms.

As indicated above, dosage forms may comprise a lubricant. Lubricants are employed to prevent adhesion of tablet material to surface of dyes and punches. Examples of lubricants include calcium stearate, glyceryl monostearate, glyceryl palmitostearate, hydrogenated castor oil, hydrogenated vegetable oil, light mineral oil, magnesium stearate, mineral oil, polyethylene glycol, sodium benzoate, sodium lauryl sulfate, silicon dioxide, sodium stearyl fumarate, stearic acid, talc, zinc stearate, and the like, or mixtures thereof. Lubricant can be used in an amount ranging up to to about 5.0% by weight based on a total weight of the dosage form.

As indicated above, dosage forms may comprise a glidant. Examples of glidants include silicon dioxide, colloidal silicon dioxide and calcium silicate. Glidant may be used in an amount ranging up to about 3% by weight based on a total weight of the dosage form.

As indicated above, dosage forms may also comprise a surfactant. Suitable surfactants include but not limited to sodium lauryl sulphate, polysorbate 20, polysorbate 80, polysorbate 40, polysorbate 60, Span 20, Span 40, Span 60, Span 80, and mixtures thereof. The surfactant is preferably used in an amount of from about 0.1% to about 5% by weight based on a total weight of the dosage form.

As indicated above, dosage forms may comprise a binder. The binder can be any pharmaceutically acceptable binder available in the tabletting art, such as acacia, alginic acid, carbomer, carboxymethylcellulose sodium, dextrin, ethylcellulose, gelatin, guar gum, hydrogenated vegetable oil (type I), hydroxyethyl cellulose, hydroxypropyl cellulose, hydroxypropyl methylcellulose, liquid glucose, magnesium aluminium silicate, maltodextrin, methylcellulose, polymethacrylates, povidone, pregelatinized starch, polyethylene glycol (PEG), sodium alginate, starch, zein, and the like, or mixtures thereof. Binder may be used in an amount ranging from about 1% to about 10% by weight based on a total weight of the dosage form.

The coloring agent may be any pharmaceutically acceptable coloring agent available in the tableting art. In one embodiment, the coloring agent is FD&C red aluminum lake. Coloring agent may be used in pharmaceutical dosage form in an amount ranging from about 0.05% to about 0.5% by weight based on a total weight of the dosage form.

In an embodiment, dosage forms comprise from about 1% to about 99% (w/w) bonded microparticulates (comprising a pharmaceutically active ingredient and a bonding agent) and from about 0.5% to about 60% (w/w) pharmaceutically acceptable excipients.

In an embodiment, the present disclosure is directed to a tablet comprising the bonded microparticulates of the present disclosure, wherein the bonded microparticulates comprise a solid particulate material and a bonding agent. Advantageously, the bonded microparticulates disclosed herein are capable of being compressed into tablets that exhibit desirable hardness and friability while the tablets exhibit an immediate release. The methods disclosed herein prepare bonded microparticulates that have excellent compressibility and processability, which prevents sticking of compressed tablet formulation to pressing dies and punches. When the bonded microparticulates disclosed herein are compressed into a tablet, the tablet exhibits a friability of not more than about 1%. In certain embodiments, the compresses tablets exhibit a friability of not more than about 0.5%.

Another embodiment of the present invention is that the method of preparing dosage form comprising bonded microparticulates does not involve any drying or milling step. The bonded microparticulates can be directly compressed or filled in capsule or can be mixed with pharmaceutically inactive ingredients and then compressed or filled in capsule. The process of preparing dosage form from bonded microparticulate is unique, uniform, reproducible, less costly and requires minimum steps and requires no special or costly ingredients.

The bonded microparticulates are converted to finished dosage form such as regular tablet, of any shape or form, fast dissolving tablet, chewable or chewmelts, fast dissolving beads, or capsule by a) Compression, b) Compactability, into finished dosage tablets with low friability, compactability and hardness. The compression force can be selected based on the type/model of press, what physical properties are desired for the tablets (e.g., desired hardness, friability, etc.), the desired tablet appearance and size, and the like. The size of the tablet can be any desirable size corresponding to an appropriately sized molds, dies and punches. Hardness is a term used in the art to describe the diametral breaking strength (DTS) as measured by conventional pharmaceutical hardness testing equipment. Typically, the compression force applied is such that the compressed tablets have a hardness of at least about 1 kp and a maximum hardness of about 30 kp. These compressed tablets generally provide sufficient hardness and strength to be packaged, shipped or handled by user. If desired, a higher compression force can be applied to the tablet to increase the tablet hardness.

According to embodiments of the present disclosure, tablet may be in a variety of different shapes. Exemplary tablet shapes include tablet shapes formed from compression tooling shapes described by “The Elizabeth Companies Tablet Design Training Manual” (Elizabeth Carbide Die Co., Inc., p. 7 (Mckeesport, Pa.) (incorporated herein by reference)

In various embodiments, parameters of tablets such as friability, hardness, thickness, and disintegration time, dissolution profile, can be measured to ensure the results meet the prerequisites of established acceptance criteria. Further adjusting the number of excipients in the dosage form, desired release and disintegration time can be achieved. Friability test is done as per USP42 NF-37 general chapter <1217>, dissolution as per dissolution Methods USP42 NF-37 general chapter <711> and disintegration as per disintegration test methods USP NF-37 general chapter <701>.

While the foregoing description discloses various embodiments of the disclosure, other and further embodiments of the invention may be devised without departing from the basic scope of the disclosure. The invention is not limited to the described embodiments, versions or examples, which are included to enable a person having ordinary skill in the art to make and use the invention when combined with information and knowledge available to the person having ordinary skill in the art.

### EXAMPLES

The present disclosure is further explained in the form of the following examples. However, it is to be understood that such examples are merely illustrative and are not to be taken as limitations upon the scope of the disclosure. Various changes and modifications to the disclosed embodiments will be apparent to those skilled in the art. Such changes and modifications may be made without departing from the scope of the disclosure.

### Example 1: BMPX of Diabetic Drug Metformin HCL

Metformin HCl is a fine powder, hydrophilic, low density solid particulate material Metformin HCl BMPX were prepared according to the components and amounts shown in Table 1-A.

TABLE 1-A
Metformin HCl BMPX

| Components | | Ex-1A | Ex-1B | Ex-1C | Ex-1D |
|---|---|---|---|---|---|
| | Pharmaceutically active ingredient | Quantity (% w/w) | | | |
| Solid | Metformin HCl | 88.00 | 88.00 | 88.00 | 89.00 |
| particulate | Form - white powder | | | | |
| material | Density- 0.45(low density) | | | | |
| | Morphology -Fine Powder | | | | |
| | Solubility- highly soluble in water | | | | |
| | Particle size- 149 microns | | | | |
| | Melting point - 223-226° C. | | | | |
| | Therapeutic Category - Diabetic agent | | | | |
| Bonding | Polymer (A)- Polyethylene glycol 8000 | 12.00 | — | — | — |
| agent | (PEG 8000) | | | | |
| | Form - white to off-white powder | | | | |
| | Melting point: 65° C. | | | | |
| | Boiling Point: 250° C. | | | | |
| | Density: 1.125 g/cm3 | | | | |
| | Emulsifier (B)- Mono Glyceride made | — | 12.00 | — | 9.00 |
| | from totally hydrogenated palm oil (90% | | | | |
| | Mono Glyceride) | | | | |
| | Form - beads | | | | |
| | Melting point: 65° C. | | | | |
| | Fat - Hydrogenated soya bean oil - | — | — | 12.00 | — |
| | white powder | | | | |
| | Melting point: 66.8° C. | | | | |
| | Emulsifier- Mono Glyceride made from | — | — | — | 2.00 |
| | totally hydrogenated palm oil (90% Mono | | | | |
| | Glyceride) + Distilled Water (D) | | | | |
| | Total | 100.00 | 100.00 | 100.00 | 100.00 |

### Procedure A for Metformin HCl BMPX With Bonding Agents A, B, C:

Metformin HCL was weighed and screened through Mesh #30. The bonding agent (A, B, C) was softened by heating in a Stainless-steel container with heating pad at a temperature of 55° C. for 20 mins. ½ part of dry screened Metformin HCl powder was added slowly to the softened bonding agent and mixed. Then remaining ½ part was added and mixed. The temperature was maintained at 60° C. and mixing was continued for further 12 min. Later the temperature was reduced to 42° C. and mixing was continued with reduced speed for further 8 min resulting in Metformin HCL BMPX. The prepared Metformin HCl BMPX was cooled at room temperature in plastic trays and then screened through the #20, #100.

### Procedure B for Metformin HCL BMPX With Bonding Agent D:

Metformin HCL was weighed and screened through Mesh #30. The bonding agent (B) was softened by heating in a Stainless-steel container with heating pad at a temperature of 55° C. for 15 mins. Warm Distilled Water (D) was added with continuous mixing for 5 Mins. ½ part of Dry screened Metformin HCl powder was added slowly and mixed to the softened bonding agent. Then the remaining ½ part was added and mixed. The total time taken for addition was 12 mins. The temperature was maintained at 60° C. and mixing was continued for further 12 min. The temperature was reduced to 42° C. and mixing with reduced speed was done for 8 min resulting in Metformin HCL BMPX. The prepared Metformin HCl BMPX was cooled at room temperature in plastic trays and then screened through the #20, #100.

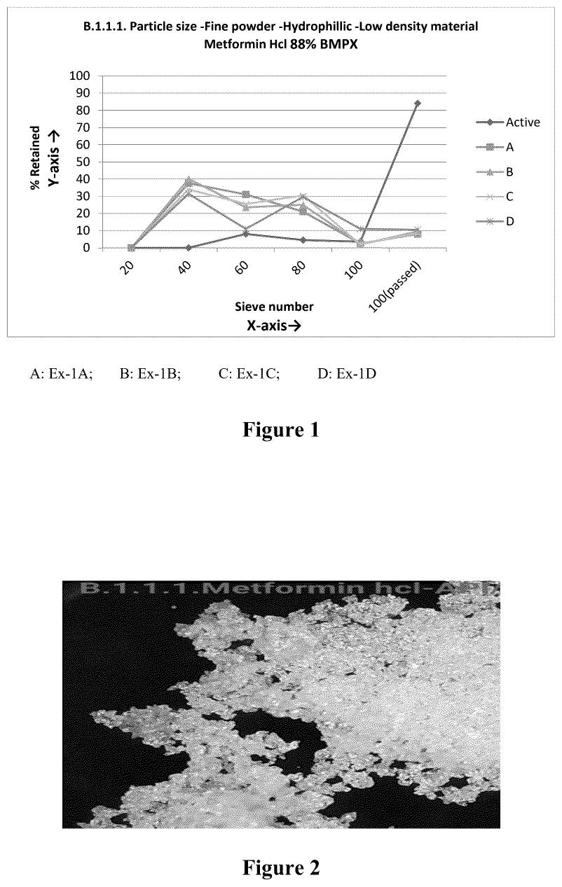

Table 1-B shows sieve analysis, bulk density and tapped density of the BMPX of Metformin HCl and Metformin HCl active. FIG. 1 shows comparison of particle size by sieve analysis for BMPX of Metformin HCl with Bonding Agents A, B, C, D.

TABLE 1-B
Sieve Analysis of Active and BMPX of Metformin HCl

| Sieve number | | Ex-1A | Ex-1B | Ex-1C | Ex-1D |
|---|---|---|---|---|---|
| (microns) | Active | Bonding Agent A | Bonding Agent B | Bonding Agent C | Bonding Agent D |
| 20(850) | 0 | 0 | 0 | 0 | 0 |
| 40(400) | 0 | 37.5 | 40 | 34 | 31.5 |
| 60(250) | 8 | 31 | 23.5 | 25.5 | 11 |
| 80(177) | 4.5 | 21 | 25 | 30.5 | 29.5 |
| 100(149) | 3.5 | 2.5 | 2 | 2 | 17 |
| 100 passed | 84 | 8 | 9.5 | 8.5 | 10.5 |
| Bulk | 0.35 | 0.42 | 0.47 | 0.4 | 0.53 |
| Density(g/ml) | | | | | |
| Tapped | 0.45 | 0.51 | 0.55 | 0.5 | 0.65 |
| density(g/ml) | | | | | |

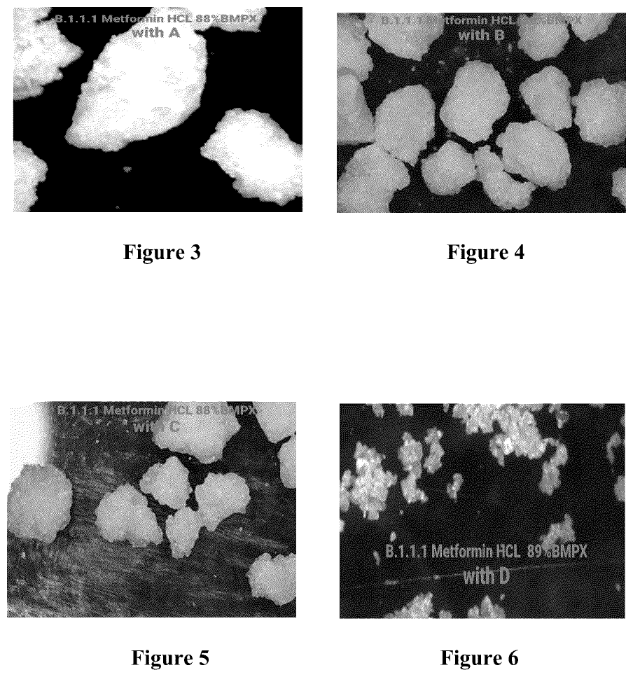

Microscopic images: FIG. 2 shows the microscopic image of Fine Powder Metformin HCL. FIG. 3 shows the microscopic image of BMPX comprising Fine Powder Metformin HCL and Bonding Agent A. FIG. 4 shows the microscopic image of BMPX comprising Fine Powder Metformin HCL and Bonding Agent B. FIG. 5 shows the microscopic image of BMPX comprising Fine Powder Metformin HCL and Bonding Agent C. FIG. 6 shows the microscopic image of BMPX comprising Fine Powder Metformin HCL and Bonding Agent D.

### Example-2-Dosage Form-Tablet Containing 500 mg of BMPX Metformin HCl

Tablet composition was prepared according to the components and amounts shown in Table-2A

TABLE
Tablet containing 500 mg of Metformin HCl BMPX

| | | EX-2A | | EX-2B | | EX-2C | | EX-2D | |
|---|---|---|---|---|---|---|---|---|---|
| | | | Qty/Dose | | Qty/Dose | % | Qty/Dose | % | Qty/Dose |
| Components | Function | % w/w | (500 mg) | % w/w | (500 mg) | w/w | (500 mg) | w/w | (500 mg) |
| BMPX of | Active | 90.95 | 568.13 | — | — | — | — | — | — |
| Metformin HCl | | | | | | | | | |
| (Ex-1A) | | | | | | | | | |
| BMPX of | Active | — | — | 90.95 | 568.13 | — | — | — | — |
| Metformin HCl | | | | | | | | | |
| (Ex-1B) | | | | | | | | | |
| BMPX of | Active | — | — | — | — | 99.0 | 567.76 | — | — |
| Metformin HCl | | | | | | | | | |
| (Ex-1C) | | | | | | | | | |
| BMPX of | Active | — | — | — | — | — | — | 90.95 | 561.75 |
| Metformin HCl | | | | | | | | | |
| (Ex-1D) | | | | | | | | | |
| Microcrystalline | Diluent | 5.00 | 31.23 | 5.00 | 31.23 | — | — | 5.00 | 30.88 |
| cellulose | | | | | | | | | |
| Sodium starch | Disintegrant | 3.00 | 18.74 | 3.00 | 18.74 | — | — | 3.00 | 18.52 |
| glycolate | | | | | | | | | |
| Colloidal | Glidant | 0.30 | 1.87 | 0.30 | 1.87 | — | — | 0.3 | 1.85 |
| silicon dioxide | | | | | | | | | |
| Magnesium | Lubricant | 0.75 | 4.68 | 0.75 | 4.68 | 1.00 | 5.735 | 0.75 | 4.63 |
| stearate | | | | | | | | | |
| | Total | 100 | 624.67 | 100 | 624.67 | 100 | 573.50 | 100 | 617.65 |

The BMPX of Metformin HCl was blended with Microcrystalline cellulose, Sodium starch glycolate, Colloidal silicon dioxide and magnesium stearate and the blend was compressed into tablet.

Table-2-B shows the characteristics of compressed tablets.

TABLE 2-B
Characteristics of compressed tablets of BMPX Metformin

| | Punch | | Compressibility | | | | | | | |
|---|---|---|---|---|---|---|---|---|---|---|
| | shape | Tablet | | Picking | Average | | Compactability | | | Disintegration |
| | Punch | Weight | | and | Weight | Weight | Tablet | Tablet | Tablet | Time |
| Eg | size | (mg) | Flow | sticking | (mg) | Variation | friability | hardness | thickness | (Minutes) |
| Ex-2A | Flat | 624.67 | Good | None | 617.25 | Within | 0.2% | 7.4 kp | 3.89 mm | 8 Min. 23 sec |
| | Round | | | | | limits | | | | |
| | shape | | | | | | | | | |
| | 13 mm | | Flow | | | | | | | |
| Ex-2B | Flat | 624.67 | Good | None | 623.35 | Within | 0.2% | 3.5 kp | 3.85 mm | 22 Min. 16 sec |
| | Round | | Flow | | | limits | | | | |
| | shape | | | | | | | | | |
| | 13 mm | | | | | | | | | |
| Ex-2C | Flat | 573.50 | Good | None | 572.90 | Within | 0.3% | 3.1 kp | 3.62 mm | 105 mins. 3 sec |
| | Round | | Flow | | | limits | | | | |
| | shape | | | | | | | | | |
| | 13 mm | | | | | | | | | |
| Ex-2D | Flat | 617.65 | Good | None | 607.8 | Within | 0.4% | 2.4 kp | 3.43 mm | 7 Mins. 12 sec |
| | Round | | Flow | | | limits | | | | |
| | shape | | | | | | | | | |
| | 13 mm | | | | | | | | | |

*Disintegration time of Ex-2C could be modified by changing the quantity of excipients.

Moisture Content: Moisture content was determined for Metformin HCl Active, BMPX Metformin and BMPX Metformin 500 mg Tablets by using the Infra Red Moisture Balance. The Moisture content data are shown in Table-2-C.

TABLE 2-C
Moisture content

| Example | Moisture Content |
|---|---|
| Active | 0.05% |
| Ex-1A | 0.05% |
| Ex-1B | 0.05% |
| Ex-1C | 0.05% |
| Ex-2A | 0.05% |
| Ex-2B | 0.05% |
| Ex-2C | 0.05% |

Result: No change in Moisture content of BMPX Metformin and BMPX Metformin 500 mg tablets was observed, when compared to Metformin active Ingredient.

### Example 3: BMPX of Cardio Vascular Drug Niacin

Niacin is a fine powder, hydrophilic, low density solid particulate material.

Niacin BMPX were prepared according to the components and amounts shown in Table-3-A.

TABLE 3-A
BMPX of Cardio Vascular drug Niacin

| Components | | Ex-3A | Ex-3B | Ex-3C |
|---|---|---|---|---|
| | Pharmaceutically active ingredient | Quantity (% w/w) | | |
| Solid | Niacin | 86.00 | 86.00 | 86.00 |
| particulate | Form - white powder | | | |
| material | Density- 0.41 (low density) | | | |
| | Morphology -Fine Powder | | | |
| | Solubility- soluble in water | | | |
| | Particle size- 149 microns | | | |
| | Melting point - 236-239° C. | | | |
| | Therapeutic Category - Cardio vascular | | | |
| Bonding | Polymer(A) - Polyethylene glycol 8000 | 14.00 | — | — |
| agent | (PEG 8000) | | | |
| | Form - white to off-white powder | | | |
| | Melting point: 65° C. | | | |
| | Boiling Point: 250° C. | | | |
| | Density: 1.125 g/cm3 | | | |
| | Emulsifier(B)-Monoglyceride made from | — | 14.00 | — |
| | totally hydrogenated palm oil (90% Mono | | | |
| | Glyceride) | | | |
| | Form - beads | | | |
| | Melting point: 65° C. | | | |
| | C)-Hydrogenated soya bean oil-Form - | — | — | 14.00 |
| | white powder | | | |
| | Melting point: 66.8° C. | | | |
| | Total | 100.00 | 100.00 | 100.00 |

Procedure for Niacin BMPX:

Niacin was weighed and screened through Mesh #30. The bonding agent (A, B, C) was softened by heating in a Stainless-steel container with heating pad at a temperature of 65° C. for 20 mins. ½ part of dry screened Niacin powder was added slowly to the softened liquid and mixed. Then the remaining ½ part was added and mixed. The temperature was maintained at 68° C. and mixing was continued for further 15 min. Later the temperature was reduced to 42° C. and mixing was continued with reduced speed for further 10 min resulting in the Niacin BMPX. The prepared Niacin BMPX was cooled at room temperature in plastic trays and then screened through the #20, #100.

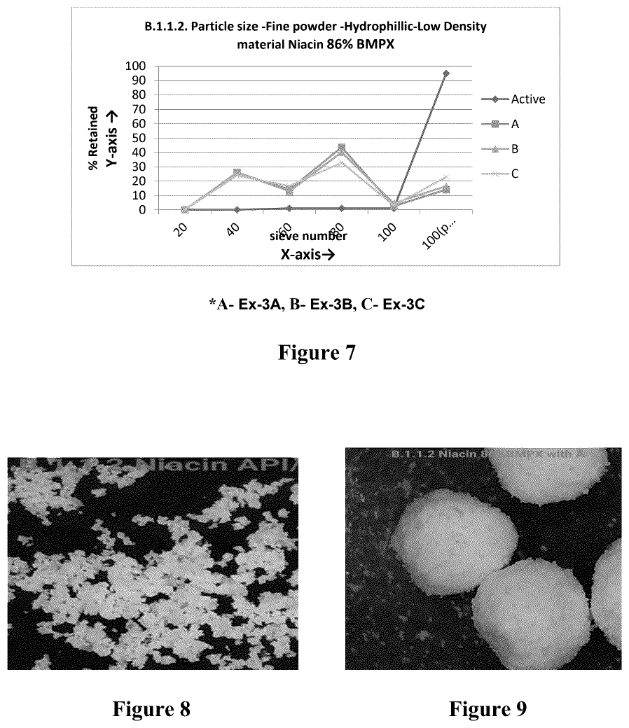

Table 3-B shows sieve analysis, bulk density and tapped density of the BMPX of Niacin. FIG. 7 shows comparison of particle size by sieve analysis for BMPX of Niacin with Bonding Agents A, B, C.

TABLE 3-A
Sieve Analysis of Active and BMPX of Niacin

| Sieve number | | Ex-3A | Ex-3B | Ex-3C |
|---|---|---|---|---|
| (microns) | Active | Bonding Agent A | Bonding Agent B | Bonding Agent C |
| 20(850) | 0 | 0 | 0 | 0 |
| 40(400) | 0 | 26 | 26 | 24 |
| 60(250) | 1 | 14 | 13 | 16.5 |
| 80(177) | 1 | 43.5 | 40 | 33 |
| 100(149) | 1 | 2.5 | 4.5 | 3 |
| 100Passed | 95 | 14 | 16.5 | 23 |
| Bulk Density | 0.27 | 0.38 | 0.3 | 0.33 |
| (g/ml) | | | | |
| Tapped | 0.41 | 0.5 | 0.5 | 0.49 |
| Density(g/ml) | | | | |

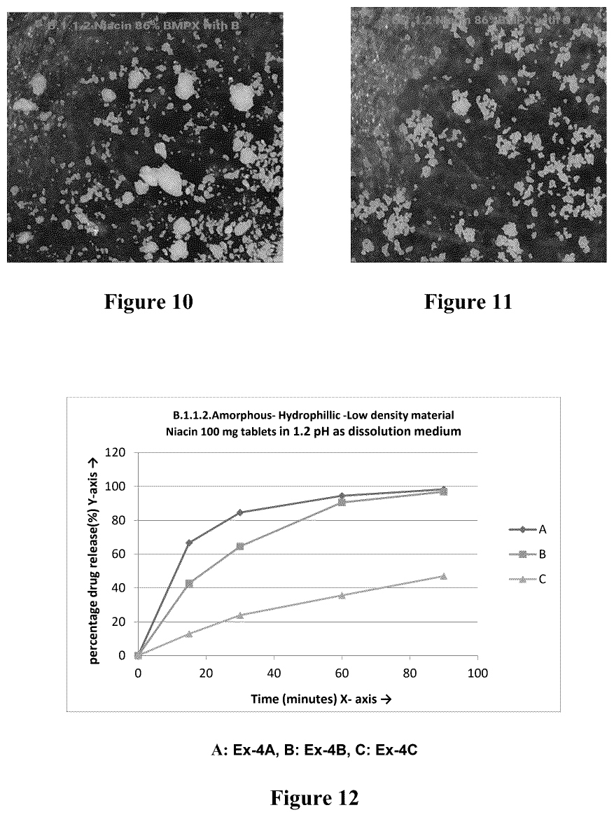

Microscopic Images: FIG. 8 shows the microscopic image of Fine Powder Niacin. FIG. 9 shows the microscopic image of Niacin BMPX comprising Fine Powder Niacin and Bonding Agent A. FIG. 10 shows the microscopic image of Niacin BMPX comprising Fine Powder Niacin and Bonding Agent B. FIG. 11 shows the microscopic image of Niacin BMPX comprising Fine Powder Niacin and Bonding Agent C.

### Example 4 Dosage Form-Tablet Containing 100 mg of BMPX Niacin

Tablet composition was prepared according to the components and amounts shown in Table-4-A

TABLE 4-A
Niacin BMPX Tablet Formulation

| | | EX-4A | | EX-4B | | EX-4C | |
|---|---|---|---|---|---|---|---|
| | | | Qty/Dose | % | Qty/Dose | % | Qty/Dose |
| Components | Function | % w/w | (100 mg) | w/w | (100 mg) | w/w | (100 mg) |
| BMPX of Niacin | Active | 90.95 | 118.23 | — | — | — | — |
| (Ex-3A) | | | | | | | |
| BMPX of Niacin | Active | — | — | 90.95 | 118.23 | — | — |
| (Ex-3B) | | | | | | | |
| BMPX of Niacin | Active | — | — | — | — | 90.75 | 116.26 |
| (Ex-3C) | | | | | | | |
| Microcrystalline | Diluent | 5.00 | 6.5 | 5.00 | 6.5 | 5.00 | 6.41 |
| cellulose | | | | | | | |
| Sodium starch | Disintegrant | 3.00 | 3.9 | 3.00 | 3.9 | 3.00 | 3.84 |
| glycolate | | | | | | | |
| Polysorbate 80 | Surfactant | — | — | — | — | 0.20 | 0.26 |
| Colloidal silicon | Glidant | 0.30 | 0.39 | 0.30 | 0.39 | 0.30 | 0.38 |
| dioxide | | | | | | | |
| Magnesium | Lubricant | 0.75 | 0.97 | 0.75 | 0.97 | 0.75 | 0.96 |
| stearate | | | | | | | |
| | Total | 100 | 130.88 | 100 | 130.88 | 100 | 128.11 |

The BMPX of Niacin were blended with above ingredients and the blend was compressed into tablet.

Table-4-B shows the characteristics of compressed tablets.

TABLE 4-B
Characteristics of compressed tablets.

| | Punch | | Compressibility | | | | | | | |
|---|---|---|---|---|---|---|---|---|---|---|
| | shape | Tablet | | Picking | Average | | Compactability | | | Disintegration |
| | Punch | Weight | | and | Weight | Weight | Tablet | Tablet | Tablet | Time |
| Example | size | (mg) | Flow | sticking | (mg) | Variation | friability | hardness | thickness | (Minutes) |
| Ex-4A | Round | 130.88 | Flow is | No picking | 129.3 | Within | 0.1% | 3.5 kp | 3.74 mm | 30 mins. 26 sec |
| | Lozenge | | good | and | | the limits | | | | |
| | shape | | | sticking is | | | | | | |
| | 6 mm | | | observed | | | | | | |
| Ex-4B | Round | 130.88 | Flow is | No picking | 131.95 | Within | 0.2% | 2.1 kp | 3.72 mm | 10 mins. 10 sec |
| | Lozenge | | good | and | | the limits | | | | |
| | shape | | | sticking is | | | | | | |
| | 6 mm | | | observed | | | | | | |
| Ex-4C | Round | 128.11 | Flow is | No picking | 122.45 | Within | 0.2% | 2.0 kp | 3.67 mm | 43 mins. 20 sec |
| | Lozenge | | good | and | | the limits | | | | |
| | shape | | | sticking is | | | | | | |
| | 6 mm | | | observed | | | | | | |

*Disintegration time of Ex4C could be modified by changing the quantity of excipients.

Moisture Content: Moisture content was determined for Niacin Active, BMPX Niacin and BMPX Niacin 100 mg tablets by using the Infra-Red Moisture Balance. The Moisture content data are shown in Table-4-C.

TABLE 4-C
Moisture content

| Example | Moisture Content |
|---|---|
| Active | 0.05% |
| Ex-3A | 0.05% |
| Ex-3B | 0.05% |
| Ex-3C | 0 |
| Ex-4A | 0.05% |
| Ex-4B | 0.05% |
| Ex-4C | 0.05% |

Result: No change in Moisture content of BMPX Niacin and BMPX Niacin 100 mg tablet was observed, when compared to Niacin active Ingredient.

Dissolution Testing: Dissolution testing of Niacin tablets was carried under following conditions.

Dissolution Parameters: Apparatus-paddle-RPM-100; Dissolution Media-1.2 pH 0.1N HCl buffer; Volume-900 ml; Detector-UV 220 nm; Temperature-37±5° C. Percentage drug release of Niacin 100 mg tablets was observed good in A, B and percentage drug release was observed less in C than the expected.

Table 4D shows the comparison of dissolution data of Niacin 100 mg tablets with Bonding Agent A, B, C in as dissolution medium. For dissolution data we plotted the graphs. FIG. 12 shows the Comparison of dissolution data of BMPX Niacin 100 mg tablets with Bonding Agent A, B, C in 1.2 pH Buffer as dissolution medium.

TABLE 4-D
(Time Vs % Drug release)

| | Ex-4A | Ex-4B | Ex-4C |
|---|---|---|---|
| Time | % Drug release | % Drug release | % Drug release |
| 15 min | 66.56 | 42.68 | 12.81 |
| 30 min | 84.47 | 64.47 | 23.84 |
| 60 min | 94.32 | 90.44 | 35.58 |
| 90 min | 98.2 | 96.71 | 46.97 |

### Example 5: BMPX of Upper Respiratory Drug Guaifenesin

Guaifenesin is a fine powder, hydrophilic, medium density solid particulate material Guaifenesin BMPX were prepared according to the components and amounts shown in Table-5-A.

TABLE 5-A
BMPX of Upper Respiratory drug Guaifenesin

| Components | | Ex-5A | Ex-5B | Ex-5C | Ex-5D |
|---|---|---|---|---|---|
| | Pharmaceutically active ingredient | Quantity (% w/w) | | | |
| Solid | Guaifenesin -Form - white powder | 88.00 | 88.00 | 88.00 | 90.00 |
| particulate | Density- 0.5(Medium density) | | | | |
| material | Morphology -Fine Powder | | | | |
| | Solubility- soluble in water | | | | |
| | Particle size- 149-177 microns | | | | |
| | Melting point - 78.5-79° C. | | | | |
| | Therapeutic Category - Upper Respiratory | | | | |
| Bonding | Polymer(A) - Polyethylene glycol 8000 (PEG | 12.00 | — | — | — |
| agent | 8000)-Form - white to off-white powder | | | | |
| | Melting point: 65° C. | | | | |
| | Boiling Point: 250° C. | | | | |
| | Density: 1.125 g/cm3 | | | | |
| | Emulsifier(B) - Monoglyceride made from totally | — | 12.00 | — | 7.00 |
| | hydrogenated palm oil (90% Mono Glyceride)- | | | | |
| | Form - beads | | | | |
| | Melting point: 65° C. | | | | |
| | Fa(C) - Hydrogenated soya bean oil-Form - white | — | — | 12.00 | — |
| | powder | | | | |
| | Melting point: 66.8° C. | | | | |
| | Emulsifier(B) - Monoglyceride made from totally | — | — | — | 3.00 |
| | hydrogenated palm oil (90% Mono Glyceride) | | | | |
| | Distilled Water (D) | | | | |
| | Total | 100.00 | 100.00 | 100.00 | 100.00 |

Procedure for Guaifenesin BMPX: BMPX comprising Guaifenesin and bonding agent (A, B, C, D) were manufactured using the procedure A & B similar to the Example 1.

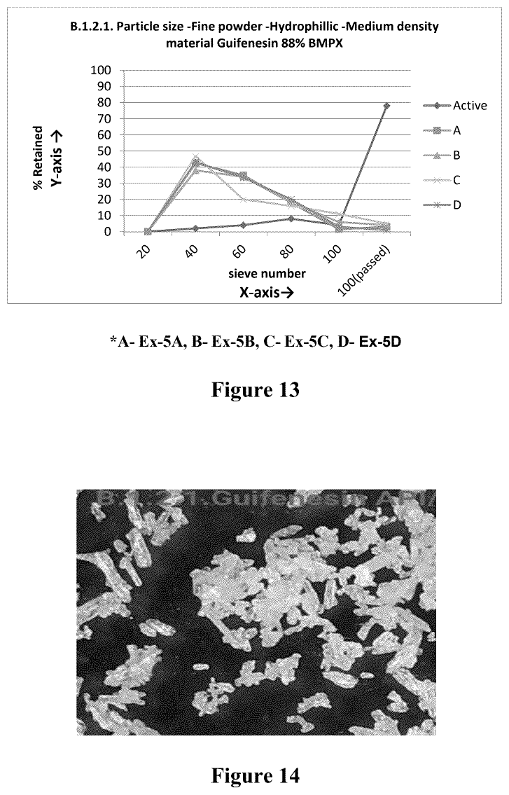

Table 5-B shows Sieve Analysis, bulk density and tapped density of the BMPX of Guaifenesin. FIG. 13 shows comparison of particle size by sieve analysis for BMPX of Guaifenesin with Bonding Agents A, B, C, D.

TABLE 5-B
Sieve Analysis of Active and BMPX Guaifenesin

| Sieve | | Ex-5A | Ex-5B | Ex-5C | Ex-5D |
|---|---|---|---|---|---|
| number | | Bonding | Bonding | Bonding | Bonding |
| (Microns) | Active | Agent A | Agent B | Agent C | Agent D |
| 20 (850) | 0 | 0 | 0 | 0 | 0 |
| 40 (400) | 2 | 42.5 | 38 | 47 | 43 |
| 60 (250) | 4 | 35 | 34 | 20 | 33.5 |
| 80 (177) | 8 | 18 | 18 | 16 | 20 |
| 100 (149) | 4 | 1.5 | 6 | 11 | 3 |
| 100 passed | 78 | 3 | 4 | 5 | 1 |
| Bulk | 0.29 | 0.54 | 0.46 | 0.4 | 0.45 |
| Density | | | | | |
| (g/ml) | | | | | |
| Tapped | 0.5 | 0.56 | 0.5 | 0.5 | 0.54 |
| density | | | | | |
| (g/ml) | | | | | |

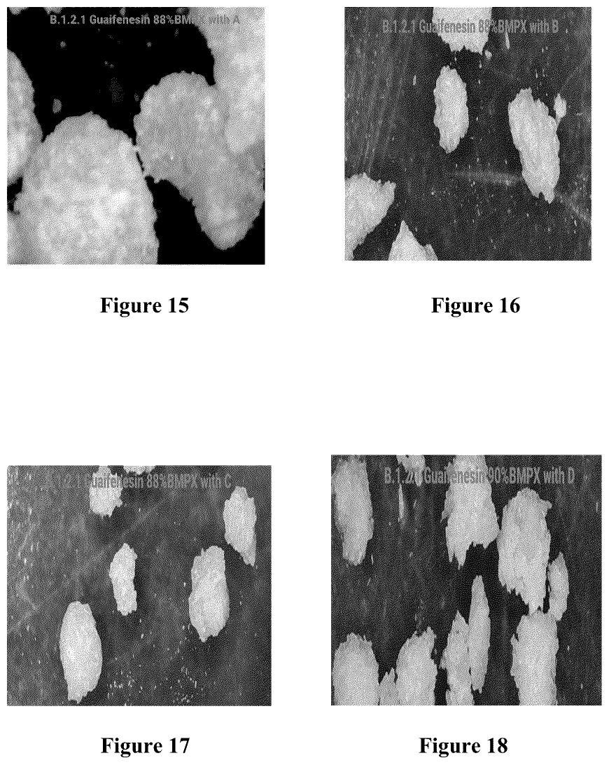

Microscopic Images: FIG. 14 shows the microscopic image of Fine Powder Guaifenesin. FIG. 15 shows the microscopic image of BMPX comprising Fine Powder Guaifenesin and Bonding Agent A. FIG. 16 shows the microscopic image of BMPX comprising Fine Powder Guaifenesin and Bonding Agent B. FIG. 17 shows the microscopic image of BMPX comprising Fine Powder Guaifenesin and Bonding Agent C. FIG. 18 shows the microscopic image of BMPX comprising Fine Powder Guaifenesin and Bonding Agent D.

### Example 6 Dosage Form-Tablet Containing 400 mg of BMPX of Guaifenesin

Tablet composition were prepared according to the components and amounts shown in Table-6-A

TABLE 6-A
BMPX of Guaifenesin Tablet Formulation

| | | EX-6A | | EX-6B | | EX-6C | | EX-6D | |
|---|---|---|---|---|---|---|---|---|---|
| Com- | Func- | % | Qty/Dose | % | Qty/Dose | % | Qty/Dose | % | Qty/Dose |
| ponents | tion | w/w | (400 mg) | w/w | (400 mg) | w/w | (400 mg) | w/w | (400 mg) |
| BMPX | Active | 90.95 | 454.48 | — | — | — | — | — | — |
| of | | | | | | | | | |
| Guaifenesin | | | | | | | | | |
| (Ex-5A) | | | | | | | | | |
| BMPX | Active | — | — | 90.95 | 454.48 | — | — | — | — |
| of | | | | | | | | | |
| Guaifenesin | | | | | | | | | |
| (Ex-5B) | | | | | | | | | |
| BMPX | Active | — | — | — | — | 99.00 | 454.41 | — | — |
| of | | | | | | | | | |
| Guaifenesin | | | | | | | | | |
| (Ex-5C) | | | | | | | | | |
| BMPX | Active | — | — | — | — | — | — | 90.95 | 222.10 |
| of | | | | | | | | | |
| Guaifenesin | | | | | | | | | |
| (Ex-5D) | | | | | | | | | |
| Microcry | Diluent | 5.00 | 24.99 | 5.00 | 24.99 | — | — | 5.00 | 24.43 |
| stalline | | | | | | | | | |
| cellulose | | | | | | | | | |
| Sodium | Dis- | 3.00 | 14.99 | 3.00 | 14.99 | — | — | 3.00 | 14.66 |
| starch | integ- | | | | | | | | |
| glycolate | rant | | | | | | | | |
| Ploysor | Sur- | — | — | — | — | — | — | — | — |
| bate-80 | factant | | | | | | | | |
| Polyplas | | — | — | — | — | — | — | — | — |
| done 80 | | | | | | | | | |
| Colloidal | Glidant | 0.30 | 1.50 | 0.30 | 1.50 | — | — | 0.30 | 1.47 |
| silicon | | | | | | | | | |
| dioxide | | | | | | | | | |
| Magnesium | Lubri- | 0.75 | 3.75 | 0.75 | 3.75 | 1.00 | 4.59 | 0.75 | 3.66 |
| stearate | cant | | | | | | | | |
| | Total | 100 | 499.70 | 100 | 499.70 | 100 | 459.0 | 100 | 488.65 |

BMPX of Guaifenesin Tablet Formulation

| | | EX-6E | | EX-6F | | EX-6G | |
|---|---|---|---|---|---|---|---|
| Com- | Func- | % | Qty/Dose | % | Qty/Dose | % | Qty/Dose |
| ponents | tion | w/w | (400 mg) | w/w | (400 mg) | w/w | (400 mg) |
| BMPX | Active | 81.95 | 454.49 | — | — | — | — |
| of | | | | | | | |
| Guaifenesin | | | | | | | |
| (Ex-5A) | | | | | | | |
| BMPX | Active | — | — | 81.95 | 454.49 | — | — |
| of | | | | | | | |
| Guaifenesin | | | | | | | |
| (Ex-5B) | | | | | | | |
| BMPX | Active | — | — | — | — | 81.75 | 468.08 |
| of | | | | | | | |
| Guaifenesin | | | | | | | |
| (Ex-5C) | | | | | | | |
| BMPX | Active | — | — | — | — | — | — |
| of | | | | | | | |
| Guaifenesin | | | | | | | |
| (Ex-5D) | | | | | | | |
| Microcry | Diluent | 10.0 | 55.46 | 10.0 | 55.46 | 10.0 | 56.07 |
| stalline | | | | | | | |
| cellulose | | | | | | | |
| Sodium | Dis- | 3.00 | 16.64 | 3.00 | 16.64 | 3.00 | 17.07 |
| starch | integ- | | | | | | |
| glycolate | rant | | | | | | |
| Ploysor | Sur- | — | — | — | — | 0.20 | 1.14 |
| bate-80 | factant | | | | | | |
| Polyplas | | 4.00 | 22.18 | 4.00 | 22.18 | 4.00 | 22.76 |
| done 80 | | | | | | | |
| Colloidal | Glidant | 0.30 | 1.66 | 0.30 | 1.66 | 0.30 | 1.17 |
| silicon | | | | | | | |
| dioxide | | | | | | | |
| Magnesium | Lubri- | 0.75 | 4.16 | 0.75 | 4.16 | 0.75 | 4.27 |
| stearate | cant | | | | | | |
| | Total | 100 | 554.60 | 100 | 554.60 | 100 | 568.90 |

The above BMPX of Guaifenesin was blended with above ingredients and then compressed into tablet.

Table-6-B shows the characteristics of compressed tablets.

TABLE 6B
Characteristics of compressed tablets.

| | Punch | | Compressibility | | | | | | | |
|---|---|---|---|---|---|---|---|---|---|---|
| | shape | Tablet | | Picking | Average | | Compactability | | | |
| | Punch | Weight | | and | Weight | Weight | Tablet | Tablet | Tablet | Disintegration |
| Example | size | (mg) | Flow | sticking | (mg) | Variation | friability | hardness | thickness | time (Minutes) |
| Ex-6A | Round | 499.70 | Flow | No picking | 498.05 | Within | 0.2% | 4.1 kp | 4.71 mm | 15 mins. 7 sec |
| | Concave | | is | and | | the limits | | | | |
| | shape | | good | sticking is | | | | | | |
| | 12 mm | | | observed | | | | | | |
| Ex-6B | Round | 499.70 | Flow | No picking | 500.15 | Within | 0.3% | 3.3 kp | 4.85 mm | 18 mins. 23 sec |
| | Concave | | is | and | | the limits | | | | |
| | shape | | good | sticking is | | | | | | |
| | 12 mm | | | observed | | | | | | |
| Ex-6C | Round | 459.00 | Flow | No picking | 461.02 | Within | 0.6% | 2.8 kp | 4.56 mm | 61 min. 5 sec |
| | Concave | | is | and | | the limits | | | | |
| | shape | | good | sticking is | | | | | | |
| | 12 mm | | | observed | | | | | | |
| Ex-6D | Round | 488.65 | Flow | No picking | 488.05 | Within | 0.2% | 2 kp | 34.78 mm | 16 mins 10 sec |
| | Concave | | is | and | | the limits | | | | |
| | shape | | good | sticking is | | | | | | |
| | 12 mm | | | observed | | | | | | |
| Ex-6E | Flat | 554.60 | Flow | No picking | 551.05 | Within | 0.2% | 4 kp | 3.45 mm | 2 mins. 56 sec |
| | Round | | is | and | | the limits | | | | |
| | shape | | good | sticking is | | | | | | |
| | 13 mm | | | observed | | | | | | |
| Ex-6F | Flat | 554.60 | Flow | No picking | 549.47 | Within | 0.6% | 2.5 kp | 3.44 mm | 2 mins. 27 sec |
| | Round | | is | and | | the limits | | | | |
| | shape | | good | sticking is | | | | | | |
| | 13 mm | | | observed | | | | | | |
| Ex-6G | Flat | 568.90 | Flow | No picking | 562.95 | Within | 0.3% | 3.1 kp | 3.54 mm | 2 mins. 42 sec |
| | Round | | is | and | | the limits | | | | |
| | shape | | good | sticking is | | | | | | |
| | 13 mm | | | observed | | | | | | |

Moisture Content: Moisture content was determined for Guaifenesin Active, Guaifenesin BMPX and BMPX Guaifenesin 400 mg tablet using the Infra Red Moisture Balance. The Moisture content data are shown in Table-6-C.

TABLE 6-C
Moisture content

| Example | Moisture Content |
|---|---|
| Active | 0.05% |
| Ex-5A | 0.05% |
| Ex-5B | 0.05% |
| Ex-5C | 0.05% |
| Ex-6A | 0.05% |
| Ex-6B | 0.05% |
| Ex-6C | 0.05% |
| Ex-6E | 0.05% |
| Ex-6F | 0.05% |
| Ex-6G | 0.05% |

Result: No change in Moisture content of BMPX Guaifenesin and BMPX Guaifenesin 400 mg tablet was observed, when compared to Guaifenesin active ingredient.

### Example 7: BMPX of Cardio Vascular Drug Amlodipine Besylate

Amlodipine Besylate is a fine powder, hydrophobic, low density solid particulate material. Amlodipine Besylate BMPX were prepared according to the components and amounts shown in Table-7-A.

TABLE 7-A
BMPX of Cardio Vascular drug Amlodipine Besylate

| Components | | Ex-7A | Ex-7B |
|---|---|---|---|
| | Pharmaceutically active ingredient | Quantity (% w/w) | |
| Solid | Amlodipine Besylate -Form - white | 86.00 | 86.00 |
| particulate | powder | | |
| material | Density- 0.4(Low density) | | |
| | Morphology -Fine Powder | | |
| | Solubility- insoluble in water | | |
| | Particle size- 177 microns | | |
| | Melting point-199° C.-201° C. | | |
| | Therapeutic Category - Cardio vascular | | |
| Bonding | Polymer (A)- Polyethylene glycol 8000 | 14.00 | — |
| agent | (PEG 8000)-Form - white to off-white | | |
| | powder | | |
| | Melting point: 65° C. | | |
| | Boiling Point: 250° C. | | |
| | Density: 1.125 g/cm3 | | |
| | Emulsifier (B)- Monoglyceride made | — | 14.00 |
| | from totally hydrogenated palm oil | | |
| | (90% Mono Glyceride)-Form - beads | | |
| | Melting point: 65° C. | | |
| | Total | 100.00 | 100.00 |

Procedure for BMPX: BMPX comprising Amlodipine Besylate and bonding agent (A, B) were manufactured using the procedure similar to Example 3.

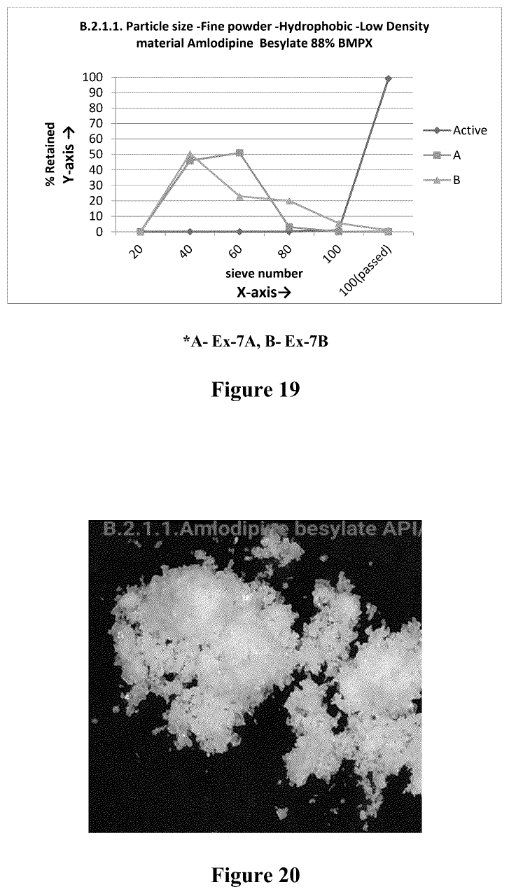

Table 7-B shows Sieve Analysis, bulk density and tapped density of the BMPX of Amlodipine Besylate. FIG. 19 shows comparison of particle size by sieve analysis for BMPX of Amlodipine Besylate with Bonding Agents A, B.

TABLE 7-B
Sieve Analysis of Active and BMPX of Amlodipine Besylate

| Sieve number | | Ex-7A | Ex-7B |
|---|---|---|---|
| (microns) | Active | Bonding Agent A | Bonding Agent B |
| 20 (850) | 0 | 0 | 0 |
| 40 (400) | 0 | 46 | 50.5 |
| 60 (250) | 0 | 51 | 23 |
| 80 (177) | 0 | 3 | 20 |
| 100 (149) | 1 | 0 | 5.5 |
| 100 passed | 99 | 0 | 1 |
| Bulk | 0.3 | 0.5 | 0.5 |
| Density(g/ml) | | | |
| Tapped | 0.45 | 0.67 | 0.55 |
| density(g/ml) | | | |

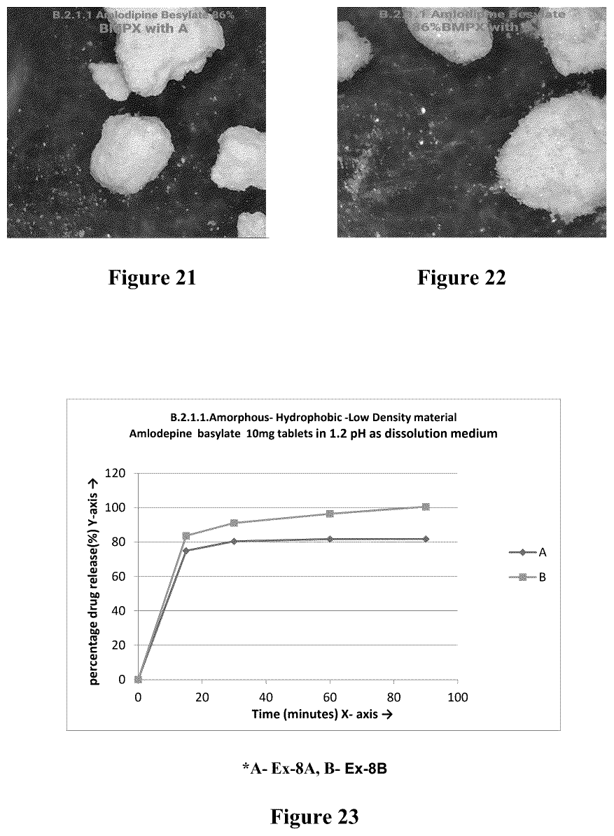

Microscopic Images: FIG. 20 shows the microscopic image of Fine Powder Amlodipine Besylate. FIG. 21 shows the microscopic image of BMPX comprising Fine Powder Amlodipine Besylate and Bonding Agent A. FIG. 22 shows the microscopic image of BMPX comprising Fine Powder Amlodipine Besylate and Bonding Agent B.

### Example 8 Dosage Form-Tablet Containing 10 mg of BMPX Amlodipine Besylate

Tablet composition was prepared according to the components and amounts shown in Table-8-A.

TABLE 8-A
BMPX Amlodipine Besylate Tablet Formulation

| | | EX-8A | | EX-8B | |
|---|---|---|---|---|---|
| | | | Qty/Dose | | Qty/Dose |
| Components | Function | Grams | (10 mg) | Grams | (10 mg) |
| BMPX Amlodipine | Active | 29.06 | 11.62 | — | — |
| Besylate (Ex-7A) | | | | | |
| BMPX of Amlodipine | Active | — | — | 29.06 | 11.62 |
| Besylate (Ex-7B) | | | | | |
| Sodium starch glycolate | Disintegrant | 3.00 | 1.20 | 3.00 | 1.20 |
| Colloidal silicon dioxide | Glidant | 0.20 | 0.08 | 0.20 | 0.08 |
| Magnesium stearate | Lubricant | 0.75 | 0.30 | 0.75 | 0.30 |
| | Total | 33.01 | 13.20 | 33.01 | 13.20 |

The BMPX of Amlodipine Besylate was blended with above ingredients and the blend was compressed into tablet.

Table-8-B shows the characteristics of compressed tablets.

TABLE 8-B
Characteristics of compressed tablets

| | Punch | | Compressibility | | | | | | | |
|---|---|---|---|---|---|---|---|---|---|---|
| | shape | Tablet | | Picking | Average | | Compactability | | | |
| | Punch | Weight | | and | Weight | Weight | Tablet | Tablet | Tablet | Disintegration |
| Example | size | (mg) | Flow | sticking | (mg) | Variation | friability | hardness | thickness | Time (Minutes) |
| Ex-8A | Round | 13.20 | Flow | No | 15.55 | Within | 0.0% | 1 kp | 1.93 mm | 5 Mins. 01 sec |
| | with four | | is | picking | | the limits | | | | |
| | multiple | | good | and | | | | | | |
| | tip 3 mm | | | sticking | | | | | | |
| | | | | is | | | | | | |
| | | | | observed | | | | | | |
| Ex-8B | Round | 13.20 | Flow | No | 14.65 | Within | 0.0% | 0.9 kp | 1.95 mm | 5 Mins. 08 sec |
| | with four | | is | picking | | the limits | | | | |
| | multiple | | good | and | | | | | | |
| | tip 3 mm | | | sticking | | | | | | |
| | | | | is | | | | | | |
| | | | | observed | | | | | | |

Moisture Content: was determined for Amlodipine Besylate Active, Amlodipine Besylate BMPX and BMPX Amlodipine 10 mg tablets by using the Infra Red Moisture Balance. The Moisture content data are shown in Table-8-C.

TABLE 8-C
Moisture content

| Example | Moisture Content |
|---|---|
| Active | 0.05% |
| Ex-7A | 0.05% |
| Ex-7B | 0.05% |
| Ex-8A | 0.05% |
| Ex-8B | 0.05% |

Result: No change in Moisture content of BMPX Amlodipine Besylate and BMPX Amlodipine Besylate 10 mg tablet was observed, when compared to Amlodipine Besylate active ingredient.

Dissolution Testing: Dissolution testing of BMPX Amlodipine Besylate tablets was carried out under the following conditions.

Dissolution Parameters: Apparatus-paddle-RPM-75; Dissolution media-1.2 pH buffer; Volume-900 ml; Detector-UV 239 nm; Temperature-37±5° C. Percentage drug release for

Amlodipine Besylate 10 mg tablets for all formulations are good.

FIG. 23 and Table 8D shows the comparison of dissolution data of BMPX Amlodipine Besylate 10 mg tablets with Bonding Agent A, B in 1.2 pH Buffer as dissolution medium.

TABLE 8-D
(Time Vs % Drug Release)

| | Ex-8A | Ex-8B |
|---|---|---|
| Time | % Drug release | % Drug release |
| 15 min | 74.83 | 83.56 |
| 30 min | 80.33 | 91.00 |
| 60 min | 81.62 | 96.31 |
| 90 min | 81.67 | 100.43 |

### Example 9: BMPX of Stimulant Drug Caffeine Anhydrous

Caffeine Anhydrous is a fine powder, hydrophobic, medium density solid particulate material Caffeine Anhydrous BMPX were prepared according to the components and amounts shown in Table-9-A.

TABLE 9-A
BMPX of Stimulant drug Caffeine Anhydrous

| Components | | Ex-9A | Ex-9B | Ex-9C |
|---|---|---|---|---|
| | Pharmaceutically active ingredient | Quantity (% w/w) | | |
| Solid | Caffeine Anhydrous-Form - white powder | 88.00 | 88.00 | 90.00 |
| particulate | Density -0.6(Medium density) | | | |
| material | Morphology -Fine Powder | | | |
| | Solubility- insoluble in water | | | |
| | Particle size- 177 microns(Not less than | | | |
| | 90% retain on 60mesh) | | | |
| | Melting point-235-237° C. | | | |
| | Therapeutic Category - Stimulant | | | |
| Bonding | Polyme- (A) - Polyethylene glycol 8000 | 12.00 | — | — |
| agent | (PEG 8000)-Form - white to off-white powder | | | |
| | Melting point: 65° C. | | | |
| | Boiling Point: 250° C. | | | |
| | Density: 1.125 g/cm3 | | | |
| | Emulsifier(B) - Mono Glyceride made | — | 12.00 | 7.00 |
| | from totally hydrogenated palm oil (90% | | | |
| | monoglyceride)- | | | |
| | Form - beads | | | |
| | Melting point: 65° C. | | | |
| | Emulsifier- Mono Glyceride made from | — | — | 3.00 |
| | totally hydrogenated palm oil (90% | | | |
| | monoglyceride) + | | | |
| | Distilled Water (C) | | | |
| | Total | 100.00 | 100.00 | 100.00 |

Procedure for BMPX: BMPX comprising Caffeine Anhydrous and bonding agent (A, B) were manufactured using the procedure similar to Example 3.

For process of BMPX with bonding Agent C we use the Procedure B similar to the Example 1.

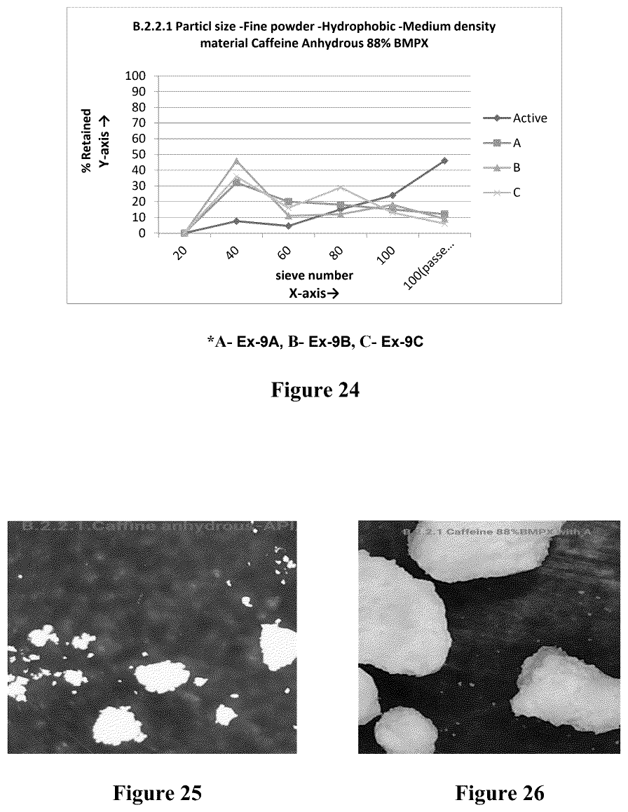

Table 9-B shows Sieve Analysis, bulk density and tapped density of the BMPX of Caffeine Anhydrous. FIG. 24 shows comparison of particle size by sieve analysis for BMPX of Caffeine Anhydrous with Bonding Agents A, B, C.

TABLE 9-B
Sieve Analysis of Active and BMPX of Caffeine Anhydrous

| | | | Ex-9A | Ex-9B | Ex-9C |
|---|---|---|---|---|---|
| Sieve number | | | Bonding | Bonding | Bonding |
| (microns) | | Active | Agent A | Agent B | Agent C |
| 20 | (850) | 0 | 0 | 0 | 0 |
| 40 | (400) | 7.5 | 32 | 46 | 36 |
| 60 | (250) | 4.5 | 20 | 11 | 16 |
| 80 | (177) | 15 | 18 | 12 | 29 |
| 100 | (149) | 24 | 15 | 18 | 13 |

| | | | | |
|---|---|---|---|---|
| 100 passed | 46 | 12 | 9 | 6 |
| Bulk | 0.4 | 0.56 | 0.62 | 0.6 |
| Density(g/ml) | | | | |
| Tapped | 0.6 | 0.71 | 0.69 | 0.65 |
| density(g/ml) | | | | |

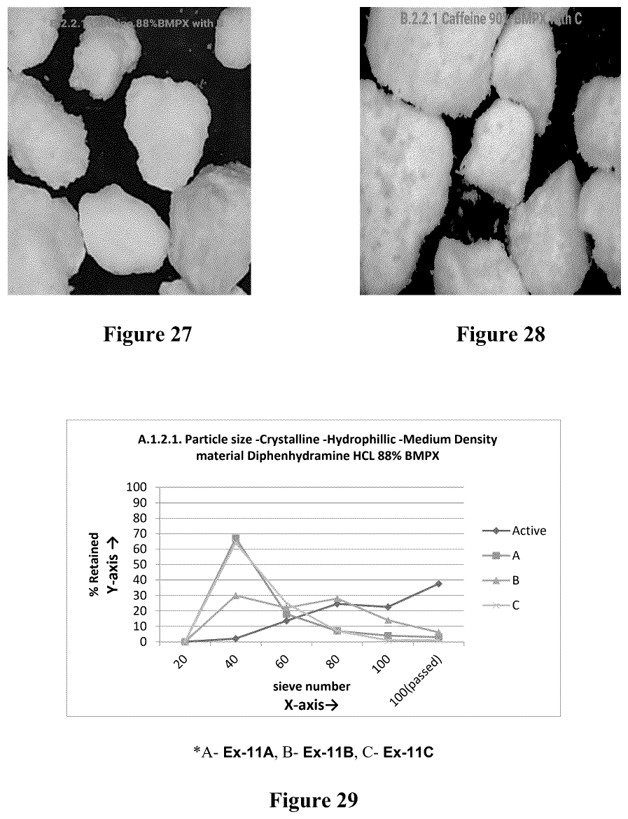

Microscopic Images: FIG. 25 shows the microscopic image of Fine Powder Caffeine Anhydrous. FIG. 26 shows the microscopic image of BMPX comprising Fine Powder Caffeine Anhydrous and Bonding Agent A. FIG. 27 shows the microscopic image of BMPX comprising Fine Powder Caffeine Anhydrous and Bonding Agent B. FIG. 28 shows the microscopic image of BMPX comprising Fine Powder Caffeine Anhydrous and Bonding Agent C.

### Example 10 Dosage Form-Tablet Containing 200 mg of BMPX Caffeine Anhydrous

Tablet composition was prepared according to the components and amounts shown in Table-10-A.

TABLE 10-A
BMPX Caffeine Anhydrous Tablet Formulation

| | | EX-10A | | EX-10B | | EX-10C | |
|---|---|---|---|---|---|---|---|
| | | | Qty/Dose | | Qty/Dose | | Qty/Dose |
| Components | Function | % w/w | (200 mg) | % w/w | (200 mg) | % w/w | (200 mg) |
| BMPX of Caffeine | Active | 99.00 | 227.2 | — | — | — | — |
| Anhydrous | | | | | | | |
| (Ex-9A) | | | | | | | |
| BMPX of | Active | — | — | 99.00 | 227.2 | — | — |
| Caffeine | | | | | | | |
| Anhydrous | | | | | | | |
| (Ex-9B) | | | | | | | |
| BMPX of | Active | — | — | — | — | 90.95 | 222.10 |
| Caffeine | | | | | | | |
| Anhydrous | | | | | | | |
| (Ex-9C) | | | | | | | |
| Magnesium | Lubricant | 1.00 | 2.29 | 1.00 | 2.29 | 0.75 | 1.8 |
| stearate | | | | | | | |
| Microcrystalline | Diluent | — | — | — | — | 5.00 | 12.2 |
| cellulose | | | | | | | |
| Sodium | Disintegrate | — | — | — | — | 3.00 | 7.3 |
| starch | | | | | | | |
| glycolate | | | | | | | |
| Colloidal | Glidant | | | | | 0.30 | 0.73 |
| silicon | | | | | | | |
| dioxide | | | | | | | |
| | Total | 100 | 229.5 | 100 | 229.5 | 100 | 244.3 |

The above BMPX of Caffeine Anhydrous were blended with above ingredients and the blend was compressed into tablet.

Table-10-B shows the characteristics of compressed tablets.

TABLE 10-B
Characteristics of compressed tablets

| | Punch | | Compressibility | | | | | | | |
|---|---|---|---|---|---|---|---|---|---|---|
| | shape | Tablet | | Picking | Average | | Compactability | | | Disintegration |
| | Punch | Weight | | and | Weight | Weight | Tablet | Tablet | Tablet | Time |
| Example | size | (mg) | Flow | sticking | (mg) | Variation | friability | hardness | thickness | (Minutes) |
| Ex-10A | Round | 229.5 | Flow | No | 228.7 | Within | 0.5% | 7 kp | 3.86 mm | 23 Mins 45 sec |
| | Concave | | is | picking | | the limits | | | | |
| | shape | | good | and | | | | | | |
| | 8 mm | | | sticking | | | | | | |
| | | | | is | | | | | | |
| | | | | observed | | | | | | |
| Ex-10B | Round | 229.5 | Flow | No | 229.2 | Within | 0.4% | 5.7 kp | 3.87 mm | 53 mins 33 sec |
| | Concave | | is | picking | | the limits | | | | |
| | shape | | good | and | | | | | | |
| | 8 mm | | | sticking | | | | | | |
| | | | | is | | | | | | |
| | | | | observed | | | | | | |
| Ex-10C | Round | 244.3 | Flow | No | 244.5 | Within | 0.4% | 7.6 kp | 3.85 mm | 17 mins 24 sec |
| | Concave | | is | picking | | the limits | | | | |
| | shape | | good | and | | | | | | |
| | 8 mm | | | sticking | | | | | | |
| | | | | is | | | | | | |
| | | | | observed | | | | | | |

*Disintegration time of Ex10B could be modified by changing the quantity of excipients.

Moisture Content: Moisture content was determined for Caffeine Anhydrous Active, Caffeine Anhydrous BMPX and BMPX Caffeine Anhydrous 200 mg tablets using the Infra-Red Moisture Balance. The Moisture content data are shown in Table-10-C.

TABLE 10-C
Moisture content

| Example | Moisture Content |
|---|---|
| Active | 0.05% |
| Ex-9A | 0.05% |
| Ex-9B | 0.05% |
| Ex-10A | 0.05% |
| Ex-10B | 0.05% |

Result: No change in Moisture content of BMPX Caffeine Anhydrous and BMPX Caffeine Anhydrous 200 mg tablet was observed, when compared to Caffeine Anhydrous active ingredients.

### Example 11: BMPX of Allergy/Sleep Drug Diphenhydramine HCL

Diphenhydramine HCl is a crystalline, hydrophilic, medium density, solid particulate material Diphenhydramine HCl BMPX were prepared according to the components and amounts shown in Table-11-A.

TABLE 11-A
BMPX of Allergy/Sleep drug Diphenhydramine HCL

| Components | | Ex-11A | Ex-11B | Ex-11C |
|---|---|---|---|---|
| | Pharmaceutically active ingredient | Quantity (% w/w) | | |
| Solid | Diphenhydramine H-I | 88.00 | 88.00 | 90.00 |
| particulate | Form - white powder | | | |
| material | Density- 0.53 (Medium density) | | | |
| | Morhology - crystalline | | | |
| | Solubility- soluble in water | | | |
| | Particle size- 149 microns | | | |
| | Melting point-331° to 338° F. | | | |
| | Therapeutic Category - Anti allergic agent | | | |
| Bonding | Polymer(A)-Polyethylene glycol 8000 | 12.00 | — | |
| agent | (PEG 80-0) | | | |
| | Form - white to off-white powder | | | |
| | Melting point: 65° C. | | | |
| | Boiling Point: 250° C. | | | |
| | Density: 1.125 g/cm3 | | | |
| | Emulsifier(B) - Monoglyceride made | — | 12.00 | 8.00 |
| | from toally hydrogenated palm oil (90% | | | |
| | monoglyceride) | | | |
| | Form - beads | | | |
| | Melting point: 65° C. | | | |
| | Emulsifier - Monoglyceride made from | | | 2.00 |
| | totally hydrogenated palm oil (90% | | | |
| | monoglyceride) + | | | |
| | Distilled Water (C) | | | |
| | Total | 100.00 | 100.00 | 100.00 |

Procedure for BMPX: BMPX comprising Diphenhydramine HCL and bonding agent (A, B) were manufactured using the procedure similar to Example 3.

For process of BMPX with bonding Agent C we use the Procedure B similar to the Example 1.

Table 11-B shows Sieve Analysis, bulk density and tapped density of the BMPX of Diphenhydramine HCl. FIG. 29 shows comparison of particle size by sieve analysis for BMPX of Diphenhydramine HCL with Bonding Agents A, B, C

TABLE 11-B
Sieve Analysis of Active and BMPX of Diphenhydramine HCl

| | | | Ex-11A | Ex-11B | Ex-11C |
|---|---|---|---|---|---|
| Sieve number | | | Bonding | Bonding | Bonding |
| (microns) | | Active | Agent A | Agent B | Agent C |
| 20 | (850) | 0 | 0 | 0 | 0 |
| 40 | (400) | 2 | 67 | 32 | 65 |
| 60 | (250) | 13.5 | 18 | 22 | 24 |
| 80 | (177) | 24.5 | 7 | 28 | 7 |
| 100 | (149) | 22.5 | 4 | 14 | 1 |

| | | | | |
|---|---|---|---|---|
| 100 passed | 37.5 | 3 | 6 | 1 |
| Bulk | 0.53 | 0.4 | 0.47 | 0.49 |
| Density(g/ml) | | | | |
| Tapped | 0.53 | 0.5 | 0.55 | 0.54 |
| density (g/ml) | | | | |

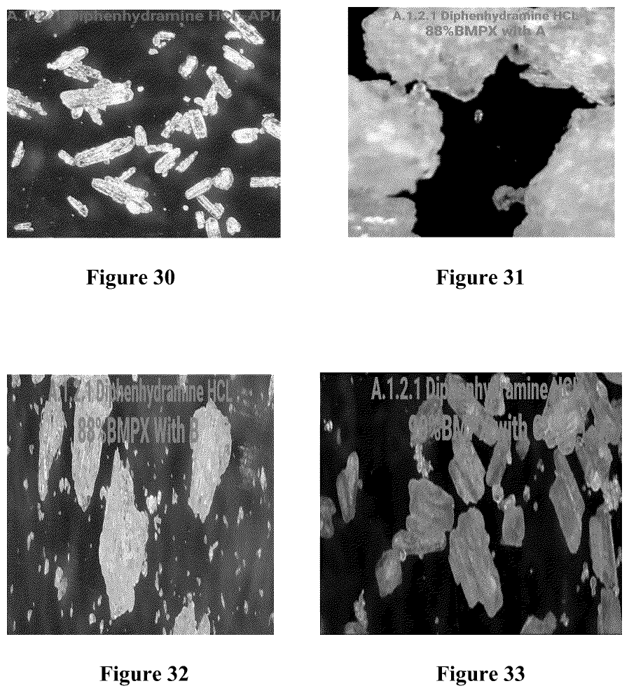

Microscopic Images: FIG. 30 shows the microscopic image of Crystalline Diphenhydramine HCL. FIG. 31 shows the microscopic image of BMPX comprising Crystalline Diphenhydramine HCL and Bonding Agent A. FIG. 32 shows the microscopic image of BMPX comprising Crystalline Diphenhydramine HCL and Bonding Agent B. FIG. 33 shows the microscopic image of BMPX comprising Crystalline Diphenhydramine HCL and Bonding Agent C.

### Example 12 Dosage Form-Tablet Containing 50 mg of BMPX Diphenhydramine HCL

Tablet composition was prepared according to the components and amounts shown in Table-12-A

TABLE 12-A
Tablet containing 50 mg of BMPX Diphenhydramine HCL

| | | EX-12A | | EX-12B | | EX-12C | |
|---|---|---|---|---|---|---|---|
| | | | Qty/Dose | | Qty/Dose | | Qty/Dose |
| Components | Function | Grams | (50 mg) | Grams | (50 mg) | Grams | (50 mg) |
| BMPX of | Active | 28.40 | 56.80 | — | — | — | — |
| Diphenhydramine | | | | | | | |
| HCL (Ex-11A) | | | | | | | |
| BMPX of | Active | — | — | 28.40 | 56.80 | — | — |
| Diphenhydramine | | | | | | | |
| HCL (Ex-11B) | | | | | | | |
| BMPX of | Active | — | — | — | — | 27.7 | 55.4 |
| Diphenhydramine | | | | | | | |
| HCL (Ex-11C) | | | | | | | |
| Sodium starch | Disintegrant | 3.00 | 6.00 | 3.00 | 6.00 | 3.00 | 6 |
| glycolate | | | | | | | |
| Calcium silicate | Glidant | 1.00 | 2.00 | 1.00 | 2.00 | 1.00 | 2.00 |
| Magnesium stearate | Lubricant | 0.75 | 1.50 | 0.75 | 1.50 | 0.75 | 1.50 |
| | Total | 33.15 | 66.30 | 33.15 | 66.30 | 32.52 | 65.04 |

The BMPX of Diphenhydramine HCL was blended with above ingredients and the blend was compressed into tablet.

Table-12-B shows the characteristics of compressed tablets.

TABLE 12-B
Characteristics of compressed tablets

| | Punch | | Compressibility | | | | | | | |
|---|---|---|---|---|---|---|---|---|---|---|
| | shape | Tablet | | Picking | Average | | Compactability | | | Disintegration |
| | Punch | Weight | | and | Weight | Weight | Tablet | Tablet | Tablet | Time |
| Example | size | (mg) | Flow | sticking | (mg) | Variation | friability | hardness | thickness | (Minutes) |
| Ex-12A | Round | 66.30 | Flow | No | 66.52 | Within the | 0.2% | 1.2 kp | 3.25 mm | 2 mins. 2 sec |
| | Concave | | is | picking | | limits | | | | |
| | shape | | good | and | | | | | | |
| | 5 mm | | | sticking | | | | | | |
| | | | | is | | | | | | |
| | | | | observed | | | | | | |
| Ex-12B | Round | 66.30 | Flow | No | 66.26 | Within the | 0.3% | 1.0 kp | 3.22 mm | 2 mins. 25 sec |
| | Concave | | is | picking | | limits | | | | |
| | shape | | good | and | | | | | | |
| | 5 mm | | | sticking | | | | | | |
| | | | | is | | | | | | |
| | | | | observed | | | | | | |
| Ex-12C | Round | 65.04 | Flow | No | 59.30 | Within the | 0.4% | 1 kp | 3.29 mm | 2 Mins. 34 sec |
| | Concave | | is | picking | | limits | | | | |
| | shape | | good | and | | | | | | |
| | 5 mm | | | sticking | | | | | | |
| | | | | is | | | | | | |
| | | | | observed | | | | | | |

Moisture Content: Moisture content was determined for Active, Diphenhydramine HCL BMPX and BMPX Diphenhydramine HCL 50 mg tablets by using the Infra Red Moisture Balance. The Moisture content data are shown in Table-12-C.

TABLE 12-C
Moisture content

| Example | Moisture Content |
|---|---|
| Active | 0.1% |
| Ex-11A | 0.1% |
| Ex-11B | 0.1% |
| Ex-12A | 0.1% |
| Ex-12B | 0.1% |

Result: No significant change in Moisture content of BMPX Diphenhydramine HCL and BMPX Diphenhydramine HCL tablet was observed, when compared to Diphenhydramine HCL active ingredient.

### Example 13: BMPX of Pain Relief Drug Ibuprofen

Ibuprofen is a fine powder, hydrophobic, medium density solid particulate material.

Ibuprofen BMPX were prepared according to the components and amounts shown in Table-13-A.

TABLE 13-A
BMPX of Pain Relief drug Ibuprofen

| Components | | Ex-13A |
|---|---|---|
| | Pharmaceutically active ingredient | Quantity (% w/w) |
| Solid | Ibuprofen | 88.00 |
| particulate | Form - white powder | |
| material | Density- 0.69(Medium density) | |
| | Morphology -Fine Powder | |
| | Solubility- in soluble in water | |
| | Particle size- 149-177 microns | |
| | Melting point -157° C. | |
| | Therapeutic Category - Pain Relief | |
| | Emulsifier(A) - Monoglyceride made | 12.00 |
| | from totally hydrogenated palm oil | |
| | (90% Mono Glyceride) | |
| | Form - beads | |
| | Melting point: 65° C. | |
| | Total | 100.00 |

Procedure for BMPX: BMPX comprising Ibuprofen and bonding agent (A) were manufactured using the procedure similar to Example 3.

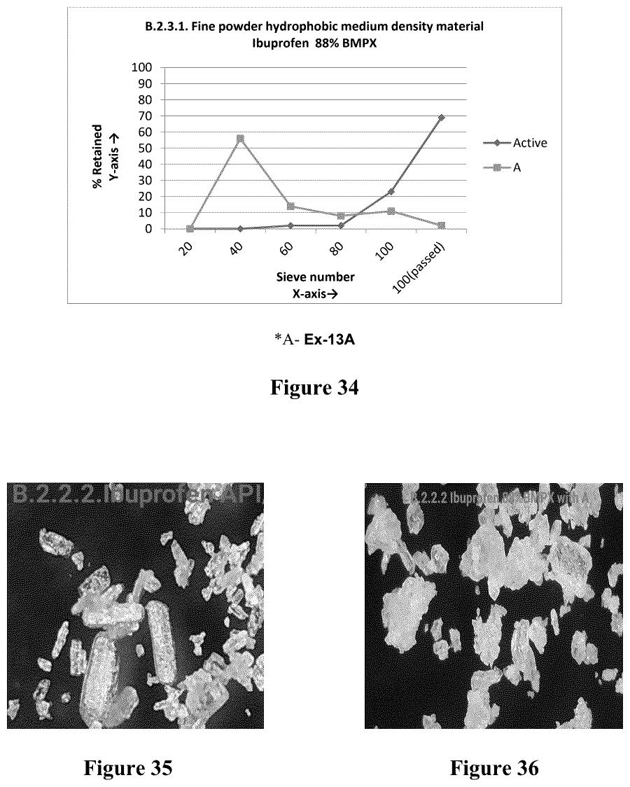

Table 13-B shows Sieve Analysis, bulk density and tapped density of the BMPX of Ibuprofen. FIG. 34 shows comparison of particle size by sieve analysis for BMPX of Ibuprofen with Bonding Agent A.

TABLE 13-B
Sieve Analysis of Active and BMPX of Ibuprofen

| | | |
|---|---|---|
| Sieve number | | Ex-13A |
| (Microns) | Active | Bonding Agent A |

| | | | |
|---|---|---|---|
| 20 | (850) | 0 | 0 |
| 40 | (400) | 0 | 56 |
| 60 | (250) | 2 | 14 |
| 80 | (177) | 2 | 8 |
| 100 | (149) | 23 | 11 |

| | | |
|---|---|---|
| 100 passed | 69 | 2 |
| Bulk Density (g/ml) | 0.45 | 0.39 |
| Tapped density (g/ml) | 0.69 | 0.46 |

Microscopic Images: FIG. 35 shows the microscopic image of Fine Powder Ibuprofen. FIG. 36 shows the microscopic image of BMPX comprising Fine Powder Ibuprofen and Bonding Agent A

### Example 14 Dosage Form-Tablet Containing 400 mg of BMPX Ibuprofen

Tablet composition was prepared according to the components and amounts shown in Table-14-A

TABLE 14-A
Tablet containing 400 mg of BMPX Ibuprofen

EX-14A

| | | | Qty/Dose |
|---|---|---|---|
| Components | Function | % w/w | (400 mg) |
| BMPX of Ibuprofen | Active | 90.95 | 454.48 |
| (Ex-13 A) | | | |
| Magnesium stearate | Lubricant | 0.75 | 3.75 |
| | Total | 100 | 499.70 |

The above BMPX of Ibuprofen were blended with Above ingredients and the blend was compressed into tablet.

Table-14-B shows the characteristics of compressed tablets

TABLE 14-B
Characteristics of compressed tablets

| | Punch | | Compressibility | | | | | | | |
|---|---|---|---|---|---|---|---|---|---|---|
| | shape | Tablet | | Picking | Average | | Compactability | | | |
| | Punch | Weight | | and | Weight | Weight | Tablet | Tablet | Tablet | Disintegration |
| Example | size | (mg) | Flow | sticking | (mg) | Variation | friability | hardness | thickness | time (Minutes) |
| Ex-14 A | Flat | 459.0 | Flow | No | 460.2 | Within | 0.3% | 2.9 kp | 4.65 mm | 90 mins. 5 sec |
| | Round | | is | picking | | the limits | | | | |
| | shape | | good | and | | | | | | |
| | 13 mm | | | sticking | | | | | | |
| | | | | is | | | | | | |
| | | | | observed | | | | | | |

*Disintegration time of Ex14A could be modified by changing the quantity of excipients.

Moisture Content: Moisture content was determined for Active, Ibuprofen BMPX and BMPX Ibuprofen 400 mg tablets by using the Infra-Red Moisture Balance. The Moisture content data are shown in Table-14-C.

TABLE 14-C
Moisture content

| Example | Moisture Content |
|---|---|
| Active | 0.1% |
| Ex-13A | 0.1% |
| Ex-14A | 0.1% |

Result: No change in Moisture content of BMPX Ibuprofen and BMPX Ibuprofen 400 mg tablets was observed, when compared to Ibuprofen active ingredient.

### Example 15: BMPX of GI Track Antacid-Oral Care Sodium Bicarbonate

Sodium Bicarbonate is a crystalline, hydrophilic, high density solid particulate material Sodium Bicarbonate BMPX were prepared according to the components and amounts shown in Table 15-A.

TABLE 15-A
BMPX of GI track Antacid-Oral Care Sodium Bicarbonate

| Components | | Ex-15A | Ex-15B | Ex-15C |
|---|---|---|---|---|
| | Pharmaceutically active ingredient | Quantity (% w/w) | | |
| Solid | Sodium Bicarbonate | 88.00 | 88.00 | 89.00 |
| particulate | Form - white powder | | | |
| material | Density -1.19 (High Density)- | | | |
| | Morphology - Crystalline | | | |
| | Solubility- soluble in water | | | |
| | Particle size- 74 microns (70.8% retain on 200#) | | | |
| | Melting point- 108° C. | | | |
| | Therapeutic Category - GI tract antacid | | | |
| | and oral care | | | |
| Bonding | Polymer (A) - Polyethylene glycol 8000 (PEG-8000) | | | |
| agent | | | | |
| | Form - white to off-white powder | 12.00 | — | |
| | Melting point: 65° C. | | | |
| | Boiling Point: 250° C. | | | |
| | Density: 1.125 g/cm3 | | | |
| | Emulsifier (B) - Monoglyceride made | — | 12.00 | 9.00 |
| | from totally hydrogenated palm oil | | | |
| | (90% monoglyc-ride) | | | |
| | Form - beads | | | |
| | Melting point: 65° C. | | | |
| | -Emulsifier - Monoglyceride made from | — | — | 2.00 |
| | totally hydrogenated palm oil | | | |
| | (90% monoglyceride) and | | | |
| | Distilled Water (C) | | | |
| | Total | 100.00 | 100.00 | 100.00 |

Procedure for BMPX: BMPX comprising Sodium Bicarbonate and bonding agent (A, B) were manufactured using the procedure similar to Example 3.

For process of BMPX with bonding Agent C we use the procedure B similar to the Example 1

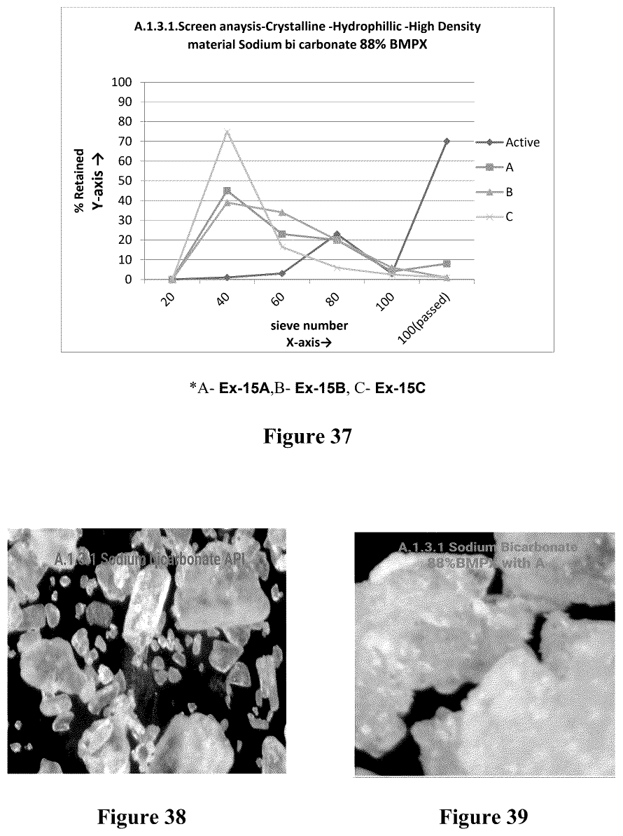

Table 15-B shows Sieve Analysis, bulk density and tapped density of the BMPX of Sodium Bicarbonate. FIG. 37 shows comparison of particle size by sieve analysis for BMPX of Sodium Bicarbonate with Bonding Agents A, B, C.

TABLE 15-B
Sieve Analysis of Active and BMPX of Sodium Bicarbonate

| | | | Ex-15A | Ex-15B | Ex-15C |
|---|---|---|---|---|---|
| Sieve number | | | Bonding | Bonding | Bonding |
| (microns) | | Active | Agent A | Agent B | Agent C |
| 20 | (850) | 0 | 0 | 0 | 0 |
| 40 | (400) | 1 | 45 | 39 | 75 |
| 60 | (250) | 3 | 23 | 34 | 16.5 |
| 80 | (177) | 23 | 20 | 20 | 6 |
| 100 | (149) | 3 | 4 | 6 | 2.5 |

| | | | | |
|---|---|---|---|---|
| 100 passed | 70 | 8 | 1 | 1 |
| Bulk | 1.04 | 1.00 | 1.69 | 0.64 |
| Density(g/ml) | | | | |
| Tapped | 1.19 | 0.92 | 0.91 | 0.86 |
| Density(g/ml) | | | | |

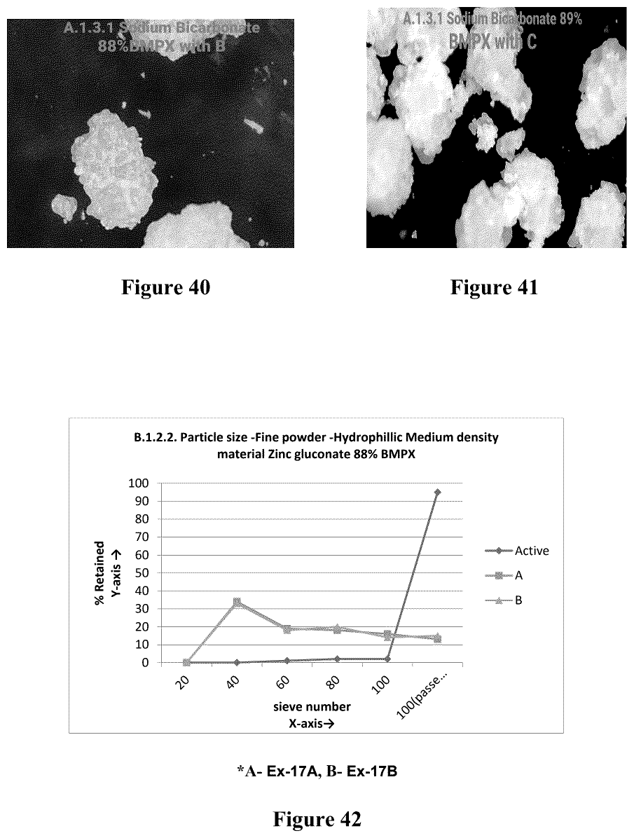

Microscopic Images: FIG. 38 shows the microscopic image of Crystalline Sodium Bicarbonate. FIG. 39 shows the microscopic image of BMPX comprising Crystalline Sodium Bicarbonate and Bonding Agent A. FIG. 40 shows the microscopic image of BMPX comprising Crystalline Sodium Bicarbonate and Bonding Agent B. FIG. 41 shows the microscopic image of BMPX comprising Crystalline Sodium Bicarbonate and Bonding Agent C.

### Example 16 Dosage Form-Tablet Containing 650 mg of Sodium Bicarbonate

Tablet composition was prepared according to the components and amounts shown in Table-16-A.

TABLE 16 A
Tablet containing 650 mg of Sodium Bicarbonate

| | | EX-16A | | EX-16B | | EX-16C | |
|---|---|---|---|---|---|---|---|
| | | % | Qty/Dose | % | Qty/Dose | % | Qty/Dose |
| Components | Function | w/w | (650 mg) | w/w | (650 mg) | w/w | (650 mg) |
| BMPX of Sodium | Active | 90.95 | 738.60 | — | — | — | — |
| Bicarbonate (Ex-15A) | | | | | | | |
| BMPX of Sodium | Active | — | — | 90.95 | 738.60 | — | — |
| Bicarbonate (Ex-15B) | | | | | | | |
| BMPX of Sodium | Active | — | — | — | — | — | — |
| Bicarbonate (Ex-15C) | | | | | | | |
| Microcrystalline cellulose | Diluent | 5.00 | 40.61 | 5.00 | 40.61 | 5.00 | 40.15 |
| Sodium starch glycolate | Disintegrant | 3.00 | 24.36 | 3.00 | 24.36 | 3.00 | 24.09 |
| Colloidal silicon dioxide | Glidant | 0.30 | 2.44 | 0.30 | 2.44 | 0.3 | 2.40 |
| Magnesium stearate | Lubricant | 0.75 | 6.09 | 0.75 | 6.09 | 0.75 | 6.02 |
| | Total | 100 | 812.10 | 100 | 812.10 | 100 | 803 |

The above BMPX of Sodium Bicarbonate was blended with above ingredients and the blend was compressed into tablet.

Table-16-B shows the characteristics of compressed tablets.

TABLE 16-B
Characteristics of compressed tablets.

| | Punch | | Compressibility | | | | | | | |
|---|---|---|---|---|---|---|---|---|---|---|
| | shape | Tablet | | Picking | Average | | Compactability | | | Disintegration |
| | Punch | Weight | | and | Weight | Weight | Tablet | Tablet | Tablet | Time |
| Example | size | (mg) | Flow | sticking | (mg) | Variation | friability | hardness | thickness | (Minutes) |
| Ex-16A | Round | 812.10 | Flow | No picking | 813 | Within | 0.2% | 9.3 kp | 4.11 mm | 14 mins. 31 sec |
| | Lozenge | | is | and | | the limits | | | | |
| | shape | | good | sticking is | | | | | | |
| | 13 mm | | | observed | | | | | | |
| Ex-16B | Round | 812.10 | Flow | No picking | 814.4 | Within | 0.2% | 8.4 kp | 4.15 mm | 70 Mins. 45 sec |
| | Lozenge | | is | and | | the limits | | | | |
| | shape | | good | sticking is | | | | | | |
| | 13 mm | | | observed | | | | | | |
| Ex-16C | Flat | 803 | Flow | No picking | 800.0 | Within | 0.3% | 4.4 kp | 4.34 mm | 22 Mins. 21 sec |
| | Round | | is | and | | the limits | | | | |
| | shape | | good | sticking is | | | | | | |
| | 13 mm | | | observed | | | | | | |

*Disintegration time of Ex16B could be modified by changing the quantity of excipients.

Moisture Content: Moisture content was determined for Active, Sodium Bicarbonate BMPX and BMPX Sodium Bicarbonate 650 mg tablets by using the Infra Red Moisture Balance. The Moisture content data are shown in Table-16-C.

TABLE 16-C
Moisture content

| Example | Moisture Content |
|---|---|
| Active | 0.1% |
| Ex-15A | 0.1% |
| Ex-15B | 0.15% |
| Ex-16A | 0.1% |
| Ex-16B | 0.1% |

Result: No change in Moisture content of BMPX Sodium Bicarbonate and BMPX Sodium Bicarbonate 650 mg tablets was observed when compared to Sodium Bicarbonate active ingredients.

### Example 17: BMPX of Pre-Cold and Supplements Zinc Gluconate

Zinc Gluconate is a fine powder, hydrophilic, medium density solid particulate material. Zinc Gluconate BMPX were prepared according to the components and amounts shown in Table-17-A.

TABLE 17-A
BMPX of Pre- cold and supplements Zinc Gluconate

| Components | | Ex-17A | Ex-17B |
|---|---|---|---|
| | Pharmaceutically active ingredient | Quantity (% w/w) | |
| Solid | Zinc Gluconate | 88.00 | 88.00 |
| particulate | Form - white powder | | |
| material | Density- 0.6(Medium density) | | |
| | Morphology -Fine Powder | | |
| | Solubility- soluble in water | | |
| | Particle size- 177 microns | | |
| | Melting point-172° C. | | |
| | Therapeutic Category - Pre- cold and | | |
| | supplements | | |
| Bonding | Polymer(A) - Polyethylene glycol | | |
| ag-nt | 8000 (-EG 8000) | | |
| | Form - white to off-white powder | 12.00 | — |
| | Melting point: 65° C. | | |
| | Boiling Point: 250° C. | | |
| | Density: 1.125 g/cm3 | | |
| | Emulsifier (B) -Monoglyceride made | — | 12.00 |
| | from totally hydrogenated palm oil | | |
| | (90% Mono Glyceride) | | |
| | Form - beads | | |
| | Melting point: 65° C. | | |
| | Total | 100.00 | 100.00 |

Procedure for BMPX: BMPX comprising Zinc Gluconate and bonding agent (A, B) were manufactured using the procedure similar to Example 3.

Table 17-B shows Sieve Analysis, bulk density and tapped density of the BMPX of Zinc Gluconate. FIG. 42 shows comparison of particle size by sieve analysis for BMPX of Zinc Gluconate with Bonding Agents A, B.

TABLE 17-B
Sieve Analysis of Active and BMPX of Zinc Gluconate

| Sieve number | | | Ex-17A | Ex-17B |
|---|---|---|---|---|
| (microns) | | Active | Bonding Agent A | Bonding Agent B |
| 20 | (850) | 0 | 0 | 0 |
| 40 | (400) | 0 | 34 | 33 |
| 60 | (250) | 1 | 19 | 18 |
| 80 | (177) | 2 | 18 | 20 |
| 100 | (149) | 2 | 16 | 14 |

| | | | |
|---|---|---|---|
| 100 passed | 95 | 13 | 15 |
| Bulk Density | 0.5 | 0.52 | 0.5 |
| (g/ml) | | | |
| Tapped density | 0.6 | 0.69 | 0.6 |
| (g/ml) | | | |

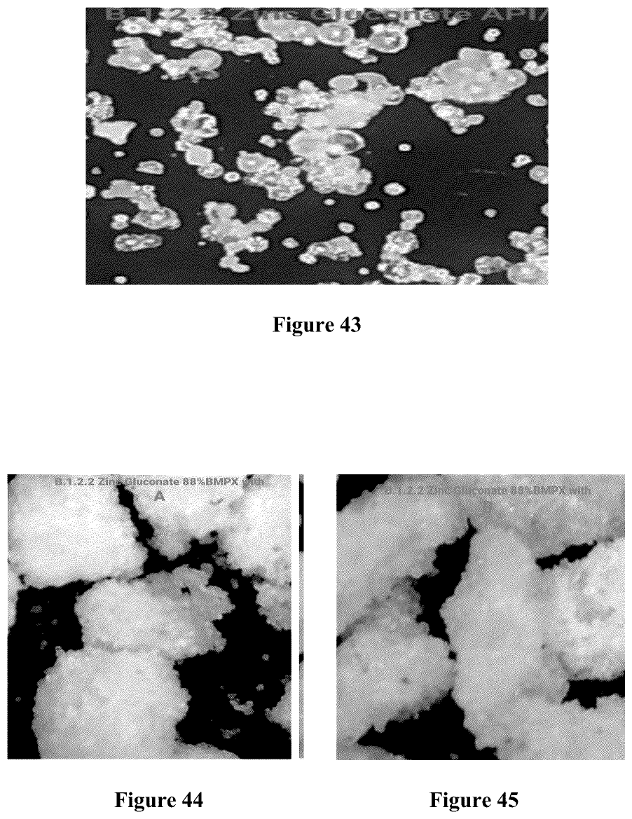

Microscopic Images: FIG. 43 shows the microscopic image of Fine Powder Zinc Gluconate. FIG. 44 shows the microscopic image of BMPX comprising Fine Powder Zinc Gluconate and Bonding Agent A. FIG. 45 shows the microscopic image of BMPX comprising Fine Powder Zinc Gluconate and Bonding Agent B.

### Example 18 Dosage Form-Tablet Containing 350 mg of BMPX Zinc Gluconate

Tablet composition was prepared according to the components and amounts shown in Table-18-A.

TABLE 18-A
Tablet containing 350 mg of BMPX Zinc Gluconate

| | | EX-18A | | EX-18B | |
|---|---|---|---|---|---|
| | | | Qty/ | | Qty/ |
| | | | Dose | | Dose |
| | | | (350 | | (350 |
| Components | Function | Grams | mg) | Grams | mg) |
| BMPX of Zinc | Active | 99.00 | 397.48 | — | — |
| Gluconate(Ex-17A) | | | | | |
| BMPX of Zinc | Active | — | — | 99.00 | 397.48 |
| Gluconate(Ex-17B) | | | | | |
| Magnesium | Lubricant | 1.00 | 4.01 | 1.00 | 4.01 |
| stearate | | | | | |
| | Total | 100 | 401.50 | 100 | 401.50 |

The above BMPX of Zinc Gluconate were blended with above ingredients and the blend was compressed into tablet.

Table-18-B shows the characteristics of compressed tablets

TABLE 18-B
Characteristics of compressed tablets

| | Punch | | Compressibility | | | | | | | | |
|---|---|---|---|---|---|---|---|---|---|---|---|
| | shape | Tablet | | Picking | Average | | Compactability | | | | Disintegration |
| | Punch | Weight | | and | Weight | Weight | Tablet | Tablet | | Tablet | Time |
| Example | size | (mg) | Flow | sticking | (mg) | Variation | friability | hardness | | thickness | (Minutes) |
| Ex-18A | Round | 401.5 | Flow | No picking | 402.2 | Within | 0.3% | 7 | kp | 3.89 mm | 12 mins. 40 sec |
| | Lozenge | | is | and | | the limits | | | | | |
| | shape | | good | sticking is | | | | | | | |
| | 10 mm | | | observed | | | | | | | |
| Ex-18B | Round | 401.5 | Flow | No picking | 400.9 | Within | 0.3% | 4.8 | kp | 3.92 mm | 44 mins. 33 sec |
| | Lozenge | | is | and | | the limits | | | | | |
| | shape | | good | sticking is | | | | | | | |
| | 10 mm | | | observed | | | | | | | |

*Disintegration time of Ex18B could be modified by changing the quantity of excipients.

Moisture Content: Moisture content was determined for Active, Zinc Gluconate BMPX and BMPX Zinc Gluconate 350 mg tablets by using the Infra Red Moisture Balance. The Moisture content data are shown in Table-18-C.

TABLE 18-C
Moisture content

| Example | Moisture Content |
|---|---|
| Active | 0.05% |
| Ex-17A | 0.05% |
| Ex-17B | 0.05% |
| Ex-18A | 0.05% |
| Ex-18B | 0.05% |

Result: No change in Moisture content of BMPX Zinc Gluconate and BMPX Zinc Gluconate 350 mg tablets was observed, when compared to Zinc Gluconate active ingredients.

### Example 19: BMPX of Prebiotic Fructo Oligosaccharide

Fructo-Oligosaccharide is a fine powder, hydrophilic, high density solid particulate material Fructo-Oligosaccharide BMPX were prepared according to the components and amounts shown in Table-19-A.

TABLE 19-A
BMPX of Prebiotic Fructo oligosaccharide

| Components | | Ex-19A | Ex-19B |
|---|---|---|---|
| | Pharmaceutically active ingredient | Quantity (% w/w) | |
| Solid | Fructo oligosaccharide | 88.00 | 88.00 |
| particulate | Form - white powder to slightly yellow | | |
| material | powder | | |
| | Density -0.75 (High Density) | | |
| | Morphology -Fine powder | | |
| | Solubility- soluble in water | | |
| | Particle size- 149 microns | | |
| | Therapeutic Category - Pre-biotics | | |
| Bonding | Polymer (A) - Polyethylene glycol 8000 | 12.00 | — |
| agent | (PEG 8000) | | |
| | Form - white to off-white powder | | |
| | Melting point: 65° C. | | |
| | Boiling Point: 250° C. | | |
| | Density: 1.125 g/cm3 | | |
| | Emulsifier (B) - Monoglyceride made | — | 12.00 |
| | from totally hydrogenated palm oil | | |
| | (90% monoglyceride) | | |
| | Form - beads | | |
| | Melting point: 65° C. | | |
| | Total | 100.00 | 100.00 |

Procedure for BMPX: BMPX comprising Fructo-Oligosaccharide and bonding agent (A, B) were manufactured using the procedure A similar to Example 1.

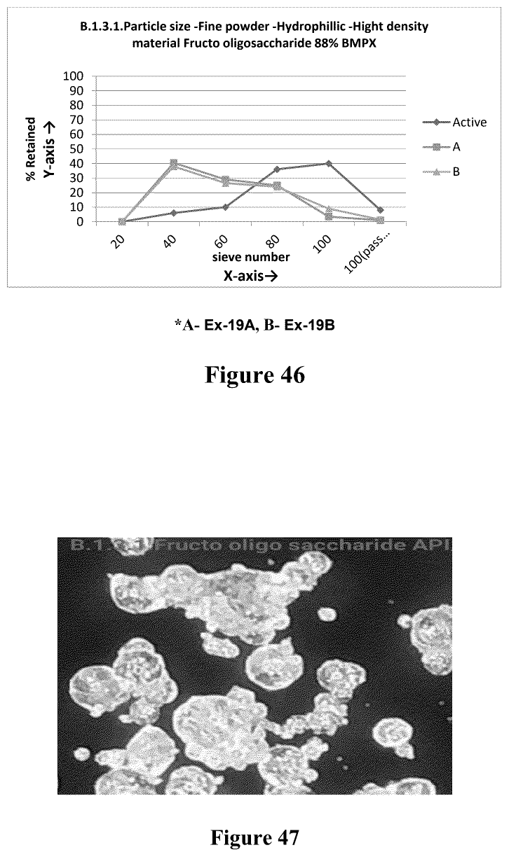

Table 19-B shows Sieve Analysis, bulk density and tapped density of the BMPX of Fructo-Oligosaccharide. FIG. 46 shows comparison of particle size by sieve analysis for BMPX of Fructo-Oligosaccharide with Bonding Agents A, B.

TABLE 19-B
Sieve Analysis of Active and BMPX of Fructo-Oligosaccharide

| Sieve number | | | Ex-19A | Ex-19B |
|---|---|---|---|---|
| (microns) | | Active | Bonding Agent A | Bonding Agent B |
| 20 | (850) | 0 | 0 | 0 |
| 40 | (400) | 6 | 40.5 | 38 |
| 60 | (250) | 10 | 29 | 26.5 |
| 80 | (177) | 36 | 25 | 24 |
| 100 | (149) | 40 | 3.5 | 9 |

| | | | |
|---|---|---|---|
| 100 passed | 8 | 1 | 1.5 |
| Bulk | 0.69 | 0.55 | 0.56 |
| Density(g/ml) | | | |
| Tapped | 0.75 | 0.46 | 0.67 |
| Density(g/ml) | | | |

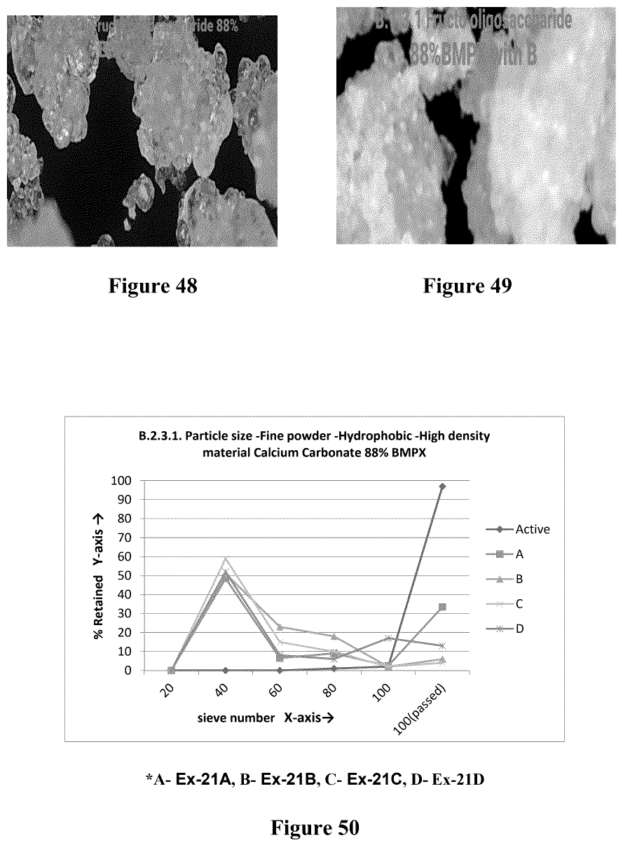

Microscopic Images: FIG. 47 shows the microscopic image of Fine Powder Fructo-Oligosaccharide. FIG. 48 shows the microscopic image of BMPX comprising Fine Powder Fructo-Oligosaccharide and Bonding Agent A. FIG. 49 shows the microscopic image of BMPX comprising Fine Powder Fructo-Oligosaccharide and Bonding Agent B.

### Example 20 Dosage Form-Tablet Containing 800 mg of BMPX Fructo-Oligosaccharide

Tablet composition was prepared according to the components and amounts shown in Table-20-A

TABLE 20-A
Tablet containing 800 mg of BMPX Fructo-Oligosaccharide

| | | EX-20A | | EX-20B | |
|---|---|---|---|---|---|
| | | | Qty/Dose | | Qty/Dose |
| Components | Function | % w/w | (800 mg) | % w/w | (800 mg) |
| BMPX of Fructo- | Active | 88.75 | 908.98 | — | — |
| Oligosaccharide | | | | | |
| (Ex-19A) | | | | | |
| BMPX of Fructo- | Active | — | — | 88.75 | 908.98 |
| Oligosaccharide | | | | | |
| (Ex-19B) | | | | | |
| Microcrystalline | Diluent | 5.00 | 51.21 | 5.00 | 51.21 |
| cellulose | | | | | |
| Sodium starch | Disin- | 3.00 | 30.73 | 3.00 | 30.73 |
| glycolate | tegrant | | | | |
| Calcium Silicate | Glidant | 1.00 | 10.24 | 1.00 | 10.24 |
| Orange Flavor | Flavor | 1.50 | 15.36 | 1.50 | 15.36 |
| Magnesium | Lubricant | 0.75 | 7.68 | 0.75 | 7.68 |
| stearate | | | | | |
| | Total | 100 | 1024.20 | 100 | 1024.20 |

The above BMPX of Fructo-Oligosaccharide were blended with above ingredients and the blend was compressed into tablet.

Table-20-B shows the characteristics of compressed tablets.

TABLE 20-B
Characteristics of compressed tablets

| | Punch | | Compressibility | | | | | | | |
|---|---|---|---|---|---|---|---|---|---|---|
| | shape | Tablet | | Picking | Average | | Compactability | | | Disintegration |
| | Punch | Weight | | and | Weight | Weight | Tablet | Tablet | Tablet | Time |
| Example | size | (mg) | Flow | sticking | (mg) | Variation | friability | hardness | thickness | (Minutes) |
| Ex-20A | Flat | 1024.20 | Flow is | No picking | 1038.4 mg | Within | 0.2% | 9.4 kp | 3.96 mm | 8 mins. 23 sec |
| | Round | | good | and | | the limits | | | | |
| | shape | | | sticking is | | | | | | |
| | 16 mm | | | observed | | | | | | |
| Ex-20B | Flat | 1024.20 | Flow is | No picking | 10372.2 | Within | 0.3% | 5.2 kp | 3.98 mm | 22 Mins. 38 sec |
| | Round | | good | and | | the limits | | | | |
| | shape | | | sticking is | | | | | | |
| | 16 mm | | | observed | | | | | | |

Moisture Content: Moisture content was determined for Active, Fructo oligosaccharide BMPX and BMPX Fructo oligosaccharide 800 mg tablets by using the Infra Red Moisture Balance. The Moisture content data are shown in Table-20-C.

TABLE 20-C
Moisture content

| Example | Moisture Content |
|---|---|
| Active | 0.5% |
| Ex-19A | 0.5% |
| Ex-19B | 0.5% |
| Ex-20A | 0.5% |
| Ex-20B | 0.5% |

Result: No change in Moisture content of Fructo oligosaccharide BMPX and BMPX Fructo oligosaccharide 800 mg tablets was observed, when compared to Active.

### Example 21: BMPX of GI-Track Antacid and Supplements Calcium Carbonate

Calcium carbonate is a fine powder, hydrophobic, high density solid particulate material Calcium carbonate BMPX were prepared according to the components and amounts shown in Table-21-A.

TABLE 21-A
BMPX of GI-track Antacid and supplements Calcium Carbonate

| Components | | Ex-21A | Ex-21B | Ex-21C | Ex-21D |
|---|---|---|---|---|---|
| | Pharmaceutically active ingredient | Quantity (% w/w) | | | |
| Solid | Calcium Carbonate | 88.00 | 90.00 | 90.00 | 89.00 |
| particulate | Form - white micro crystalline powder | | | | |
| material | Density -1.2(High Density) | | | | |
| | Morphology - Crystalline | | | | |
| | Solubility- insoluble in water | | | | |
| | Particle size- 177 microns (Not less than | | | | |
| | 90% retain on 60mesh | | | | |
| | Melting Point- 825° C. | | | | |
| | Therapeutic Category -Antacid and | | | | |
| | Calcium Supplements | | | | |
| Bonding | Polymer (A) - Polyethylene glycol 8000 | 12.00 | — | — | — |
| agent | (PEG 8000) | | | | |
| | Form - white to off-white powder | | | | |
| | Melting point: 65° C. | | | | |
| | Boiling Point: 250° C. | | | | |
| | Density: 1.125 g/cm3 | | | | |
| | Emulsifier (B) - Monoglyceride made | — | 10.00 | — | 9.00 |
| | from totally hydrogenated palm oil (90% | | | | |
| | Mono Glyceride) | | | | |
| | Form - beads | | | | |
| | Melting point: 65° C. | | | | |
| | Fat (C)- Hydrogenated soya-bean oil | — | — | 10.00 | — |
| | Form - white powder | | | | |
| | Melting point: 66.8° C. | | | | |
| | Monoglyceride made from totally | — | — | — | 2.00 |
| | hydrogenated palm oil (90% Mono | | | | |
| | Glyceride) + | | | | |
| | Distilled Water (D) | | | | |
| | Total | 100.00 | 100.00 | 100.00 | 100.00 |

Procedure for BMPX: BMPX comprising Calcium Carbonate and bonding agent (A, B, C, D) were manufactured using the procedure A & B similar to Example 1.

Table 21-B shows Sieve Analysis, bulk density and tapped density of the BMPX of Calcium Carbonate. FIG. 50 shows comparison of particle size by sieve analysis for BMPX of Calcium carbonate with Bonding Agents A, B, C, D.

TABLE 21-B
Sieve Analysis of Active and BMPX of Calcium Carbonate

| | | | Ex-21A | Ex-21B | Ex-21C | Ex-21D |
|---|---|---|---|---|---|---|
| Sieve number | | | Bonding | Bonding | Bonding | Bonding |
| (microns) | | Active | Agent A | Agent B | Agent C | Agent D |
| 20 | (850) | 0 | 0 | 0 | 0 | 0 |
| 40 | (400) | 0 | 48.5 | 60.5 | 59 | 52.5 |
| 60 | (250) | 0 | 6.5 | 19 | 15 | 8 |
| 80 | (177) | 1 | 9 | 11 | 10 | 6 |
| 100 | (149) | 2 | 2.5 | 5.5 | 2 | 17 |

| | | | | | |
|---|---|---|---|---|---|
| 100 passed | 97 | 33.5 | 4 | 4 | 13 |
| Bulk | 0.73 | 1.0 | 1.08 | 0.96 | 1.13 |
| Density(g/ml) | | | | | |
| Tapped | 1.2 | 1.25 | 1.19 | 1.00 | 1.31 |
| Density(g/ml) | | | | | |

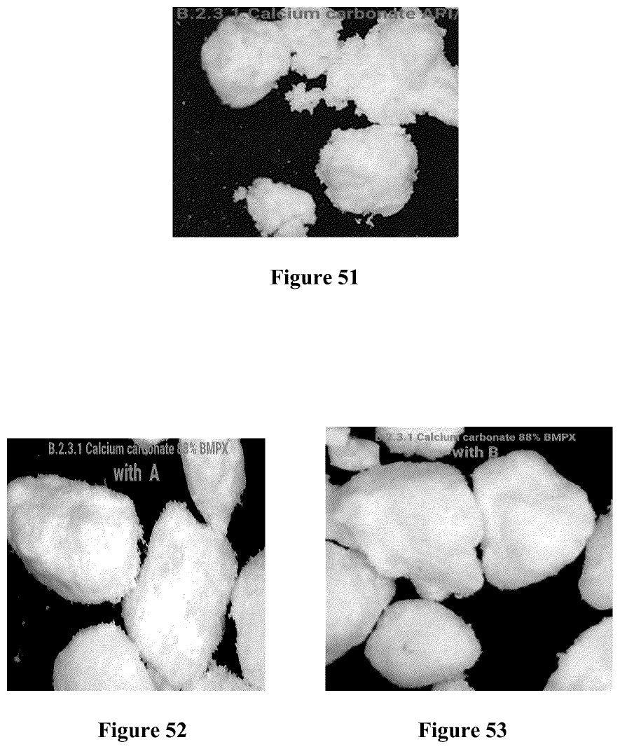

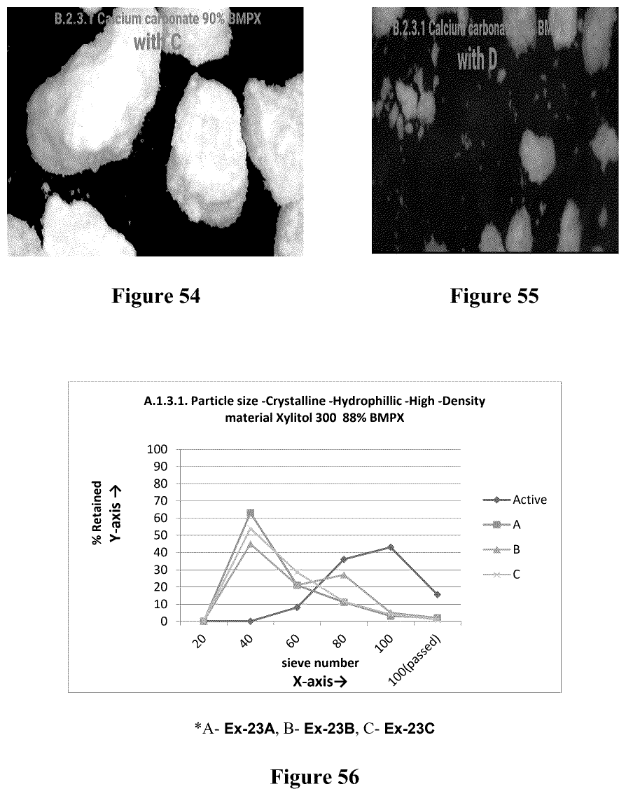

Microscopic Images: FIG. 51 shows the microscopic image of Fine powder Calcium Carbonate. FIG. 52 shows the microscopic image of BMPX comprising Fine Powder Calcium Carbonate and Bonding Agent A. FIG. 53 shows the microscopic image of BMPX comprising Fine Powder Calcium Carbonate and Bonding Agent B. FIG. 54 shows the microscopic image of BMPX comprising Fine Powder Calcium Carbonate and Bonding Agent C. FIG. 55 shows the microscopic image of BMPX comprising Fine Powder Calcium Carbonate and Bonding Agent D

### Example 22 Dosage Form-Tablet Containing 1250 mg of BMPX Calcium Carbonate

Tablet composition was prepared according to the components and amounts shown in Table-22-A.

TABLE 22-A
Tablet containing 1250 mg of BMPX Calcium carbonate

| | | EX-22A | | EX-22B | | EX-22C | | EX-22D | |
|---|---|---|---|---|---|---|---|---|---|
| | | | Qty/Dose | | Qty/Dose | | Qty/Dose | | Qty/Dose |
| Components | Function | % w/w | (1250 mg) | % w/w | (1250 mg) | % w/w | (1250 mg) | % w/w | (1250 mg) |
| BMPX of | Active | 90.96 | 1420.47 | — | — | — | — | — | — |
| Calcium | | | | | | | | | |
| Carbonate | | | | | | | | | |
| (Ex-21A) | | | | | | | | | |
| BMPX | Active | — | — | 90.75 | 1388.84 | — | — | — | — |
| Calcium | | | | | | | | | |
| Carbonate | | | | | | | | | |
| (Ex-21B) | | | | | | | | | |
| BMPX of | Active | — | — | — | — | 90.75 | 1388.84 | — | — |
| Calcium | | | | | | | | | |
| Carbonate | | | | | | | | | |
| (Ex-21C) | | | | | | | | | |
| BMPX of | Active | — | — | — | — | — | — | 90.95 | 1404.45 |
| Calcium | | | | | | | | | |
| Carbonate | | | | | | | | | |
| (Ex-21D) | | | | | | | | | |
| Micro | Diluent | 5.00 | 78.08 | 5.00 | 76.52 | 5.00 | 76.52 | 5.00 | 77.21 |
| Crystalline | | | | | | | | | |
| Cellulose | | | | | | | | | |
| Sodium | Disintegrant | 3.00 | 46.84 | 3.00 | 45.91 | 3.00 | 45.91 | 3.00 | 46.32 |
| Starch | | | | | | | | | |
| glycolate | | | | | | | | | |
| Polysorbate- | Disintegrant | — | — | 0.20 | 3.06 | 0.20 | 3.06 | 0.2 | 3.08 |
| 80 | | | | | | | | | |
| Colloidal | Glidant | 0.30 | 4.684 | 0.3 | 4.59 | 0.3 | 4.59 | 0.3 | 4.63 |
| Silicon dioxide | | | | | | | | | |
| Magnesium | Lubricant | 0.75 | 11.71 | 0.75 | 11.48 | 0.75 | 11.48 | 0.75 | 11.58 |
| Stearate | | | | | | | | | |
| | Total | 100 | 1561.70 | 100 | 1530.40 | 100 | 1530.40 | 100 | 1544.2 |

The above BMPX of Calcium carbonate was blended with above ingredients and the blend was compressed into tablet.

Table-22-B shows the characteristics of compressed tablets

TABLE 22-B
Characteristics of compressed tablets

| | Punch | | Compressibility | | | | | | | | |
|---|---|---|---|---|---|---|---|---|---|---|---|
| | shape | Tablet | | Picking | Average | | Compactability | | | | Disintegration |
| | Punch | Weight | | and | Weight | Weight | Tablet | Tablet | | Tablet | Time |
| Example | size | (mg) | Flow | sticking | (mg) | Variation | friability | hardness | | thickness | (Minutes) |
| Ex-22A | Flat | 1561.70 | Flow | No picking | 1560.4 | Within | 0.2% | 9.5 | kp | 4.10 mm | 28 Mins. 44 sec |
| | Round | | is | and | | the limits | | | | | |
| | Shape | | good | sticking is | | | | | | | |
| | 16 mm | | | observed | | | | | | | |
| Ex-22B | Flat | 1530.40 | Flow | No picking | 1532.9 | Within | 0.3% | 8.6 | kp | 3.84 mm | 40 Mins. 01 sec |
| | Round | | is | and | | the limits | | | | | |
| | Shape | | good | sticking is | | | | | | | |
| | 16 mm | | | observed | | | | | | | |
| Ex-22C | Flat | 1530.40 | Flow | No picking | 1530.05 | Within | 0.0% | 13 | kp | 3.95 mm | 79 Mins. 50 sec |
| | Round | | is | and | | the limits | | | | | |
| | Shape | | good | sticking is | | | | | | | |
| | 16 mm | | | observed | | | | | | | |
| Ex-22D | Flat | 1544.2 | Flow | No picking | 1531.7 | Within | 0.3% | 7.4 | kp | 4.03 mm | 13 Mins. 12 sec |
| | Round | | is | and | | the limits | | | | | |
| | shape | | good | sticking is | | | | | | | |
| | 16 mm | | | observed | | | | | | | |

*Disintegration time of Ex-22B & Ex-22C could be modified by changing the quantity of excipients.

Moisture Content: Moisture content was determined for Calcium Carbonate Active, Calcium Carbonate BMPX and BMPX Calcium Carbonate 1250 mg tablets by using the Infra Red Moisture Balance. The Moisture content data are shown in Table-22-C.

TABLE 22-C
Moisture content

| Example | Moisture Content |
|---|---|
| Active | 0.05% |
| Ex-21A | 0.05% |
| Ex-21B | 0.05% |
| Ex-21C | 0.05% |
| Ex-22A | 0.10% |
| Ex-22B | 0.05% |
| Ex-22C | 0.05% |

Result: No significant change of Moisture content in BMPX Calcium Carbonate and Calcium Carbonate 1250 mg tablets was observed when compared to Calcium Carbonate active ingredient.

### Example 23: BMPX of Oral Care Xylitol-300

Xylitol-300 is a crystalline, hydrophilic, high density, solid particulate material

Xylitol-300 BMPX were prepared according to the components and amounts shown in Table-23-A.

TABLE 23-A
BMPX of Oral care Xylitol-300

| Components | | Ex-23A | Ex-23B | Ex-23C |
|---|---|---|---|---|
| | Pharmaceutically active ingredient | Quantity (% w/w) | | |
| Solid | Xylitol-300 | 88.00 | 88.00 | 90.00 |
| particulate | Form - white micro crystalline powder | | | |
| material | Density -0.83 (High Density) | | | |
| | Morphology - Crystalline | | | |
| | Solubility- soluble in water | | | |
| | Particle size- 149 microns | | | |
| | Therapeutic Category -oral care | | | |
| Bonding | Polymer (A) - Polyethylene glycol 8000 | 12.00 | — | — |
| agent | (PEG 8000) | | | |
| | Form - white to off-white powder | | | |
| | Melting point: 65° C. | | | |
| | Boiling Point: 250° C. | | | |
| | Density: 1.125 g/cm3 | | | |
| | Emulsifier (B) - Monoglyceride made | — | 12.00 | 8.00 |
| | from totally hydrogenated palm oil (90% | | | |
| | Mono Glyceride) | | | |
| | Form - beads | | | |
| | Melting point: 65° C. | | | |
| | Emulsifier: Monoglyceride made from | — | — | 2.00 |
| | totally hydrogenated palm oil (90% Mono | | | |
| | Glyceride) + | | | |
| | Distilled Water (C) | | | |
| | Total | 100.00 | 100.00 | 100.00 |

Procedure for BMPX: BMPX comprising Xylitol-300 and bonding agent (A, B, C) were manufactured using the procedure A & B similar to Example 1.

Table 23-B shows Sieve Analysis, bulk density and tapped density of the BMPX of Xylitol-300. FIG. 56 shows comparison of particle size by sieve analysis for BMPX of Xylitol-300 with Bonding Agents A, B, C.

TABLE 23-B
Sieve Analysis of Active and BMPX of Xylitol-300

| | | | Ex-23A | Ex-23B | Ex-23C |
|---|---|---|---|---|---|
| Sieve number | | | Bonding | Bonding | Bonding |
| (microns) | | Active | Agent A | Agent B | Agent C |
| 20 | (850) | 0 | 0 | 0 | 0 |
| 40 | (400) | 0 | 63 | 45 | 54 |
| 60 | (250) | 7 | 21 | 21 | 28.5 |
| 80 | (177) | 35 | 11 | 27 | 11.5 |
| 100 | (149) | 42 | 3 | 5 | 4 |

| | | | | |
|---|---|---|---|---|
| 100 passed | 14.5 | 2 | 2 | 1 |
| Bulk | 0.53 | 0.5 | 0.62 | 0.53 |
| Density(g/ml) | | | | |
| Tapped | 0.83 | 0.55 | 0.71 | 0.65 |
| Density(g/ml) | | | | |

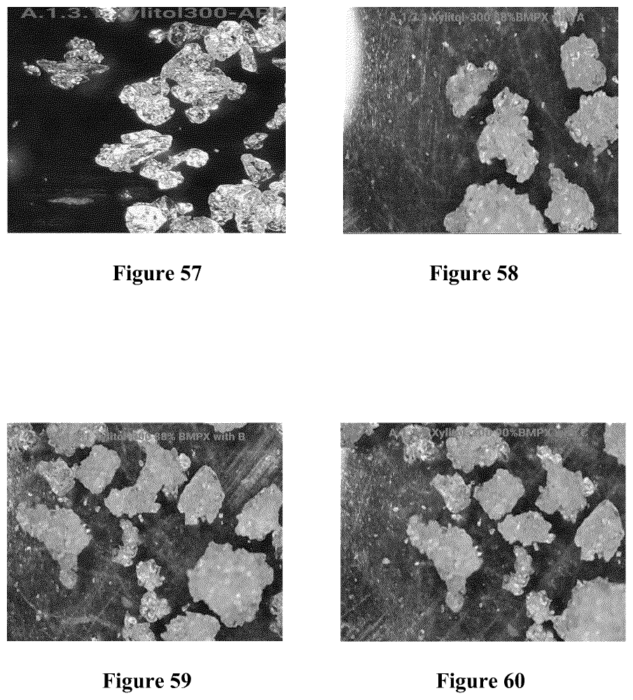

Microscopic Images: FIG. 57 shows the microscopic image of Crystalline Xylitol-300. FIG. 58 shows the microscopic image of BMPX comprising Crystalline Xylitol-300 and Bonding Agent A. FIG. 59 shows the microscopic image of BMPX comprising Crystalline Xylitol-300 and Bonding Agent B. FIG. 60 shows the microscopic image of BMPX comprising Crystalline Xylitol-300 and Bonding Agent C.

### Example 24 Dosage Form-Tablet Containing 400 mg of Xylitol-300

Tablet composition was prepared according to the components and amounts shown in Table-24-A

TABLE 24-A
Tablet containing 400 mg of Xylitol-300

| | | EX-24A | | EX-24B | | EX-24C | |
|---|---|---|---|---|---|---|---|
| | | | Qty/Dose | % | Qty/Dose | | Qty/Dose |
| Components | Function | % w/w | (400 mg) | w/w | (400 mg) | % w/w | (400 mg) |
| BMPX ofXylitol- | Active | 97.50 | 399.70 | — | — | — | — |
| 300 (Ex-23A) | | | | | | | |
| BMPX ofXylitol- | Active | — | — | 97.50 | 399.70 | — | — |
| 300 (Ex-23B) | | | | | | | |
| BMPX ofXylitol- | Active | — | — | — | — | 93.5 | 444.125 |
| 300 (Ex-23C) | | | | | | | |
| Cool mint | Flavor | 1.50 | 6.15 | 1.50 | 6.15 | — | — |
| Microcrystalline | Diluent | — | — | — | — | 5.00 | 23.75 |
| cellulose | | | | | | | |
| Calcium Silicate | Glidant | — | — | — | — | 0.5 | 2.375 |
| Magnesium | Lubricant | 1.00 | 4.10 | 1.00 | 4.10 | 1.00 | 4.75 |
| stearate | | | | | | | |
| | Total | 100 | 410.00 | 100 | 410.00 | 100 | 475 |

The BMPX of Xylitol-300 were blended with above ingredients and the blend was compressed into tablet.

Table-24-B shows the characteristics of compressed tablets.

TABLE 24-B
Characteristics of compressed tablets

| | Punch | | | Compressibility | | | | | | | | |
|---|---|---|---|---|---|---|---|---|---|---|---|---|
| | shape | Tablet | | Picking | Average | | Compactability | | | | | Disintegration |
| | Punch | Weight | | and | Weight | Weight | Tablet | Tablet | | Tablet | | Time |
| Example | size | (mg) | Flow | sticking | (mg) | Variation | friability | hardness | | thickness | | (Minutes) |
| Ex-24A | Round | 410.00 | Flow | No picking | 409.2 | Within | 0.4% | 6.1 | kp | 4.9 | mm | 4 mins. 56 sec |
| | Concave | | is | and | | the limits | | | | | | |
| | shape | | good | sticking is | | | | | | | | |
| | 10 mm | | | observed | | | | | | | | |
| Ex-24B | Round | 410.00 | Flow | No picking | 409.75 | Within | 0.4% | 3.5 | kp | 4.19 | mm | 30 Mins. 42 sec |
| | Concave | | is | and | | the limits | | | | | | |
| | shape | | good | sticking is | | | | | | | | |
| | 10 mm | | | observed | | | | | | | | |
| Ex-24C | Round | 475.00 | Flow | No picking | 474.7 | Within | 0.3% | 3.14 | kp | 4.03 | mm | 14 Mins. 41 sec |
| | Lozenge | | is | and | | the limits | | | | | | |
| | shape | | good | sticking is | | | | | | | | |
| | 11 mm | | | observed | | | | | | | | |

Moisture Content: Moisture content was determined for Active, BMPX Xylitol-300 and BMPX Xylitol-300 400 mg by using the Infra Red Moisture Balance. The Moisture content data are shown in Table-24-C.

TABLE 24-C
Moisture content

| Example | Moisture Content |
|---|---|
| Active | 0.1% |
| Ex-23A | 0.1% |
| Ex-23B | 0.1% |
| Ex-24A | 0.1% |
| Ex-24B | 0.1% |

Result: No change of Moisture content in BMPX Xylitol-300 and BMPX Xylitol-300 400 mg tablets when compared to Xylitol-300 active ingredient.

### Example 25: BMPX of SugarXylitol-90

Xylitol-90 is a fine powder, hydrophilic, high density solid particulate material

Xylitol-90 BMPX were prepared according to the components and amounts shown in Table-25-A.

TABLE 25-A
BMPX of SugarXylitol-90

| Components | | Ex-25A | Ex-25B | Ex-25C | Ex-25D |
|---|---|---|---|---|---|
| | Pharmaceutically active ingredient | Quantity (% w/w) | | | |
| Solid | Xylitol-90 | 88.00 | 88.00 | 88.00 | 90.00 |
| particulate | Form - white powder | | | | |
| material | Density -0.8(High Density) | | | | |
| | Morphology -Fine Powder | | | | |
| | Solubility- soluble in water | | | | |
| | Particle size- 90 microns | | | | |
| | Melting Point- 94.5° C. | | | | |
| | Therapeutic Category-Sugar | | | | |
| Bonding -agent | Polymer (A) - Polyethylene glycol 800- | 12.00 | — | — | — |
| | (PEG 8000) | | | | |
| | Form - white to off-white powder | | | | |
| | Melting point: 65° C. | | | | |
| | Boiling Point: 250° C. | | | | |
| | Density: 1.125 g/cm3 | | | | |
| | Emulsifier (B) - Monoglyceride made | — | 12.00 | — | 8.00 |
| | from totally hydrogenated palm oil (90% | | | | |
| | Mono Glyceride) | | | | |
| | Form - beads | | | | |
| | Melting point: 65° C. | | | | |
| | Fat (C)- Hydrogenated soya bean oil | — | — | 12.00 | — |
| | Form - white powder | | | | |
| | Melting point: 66.8° C. | | | | |
| | Monoglyceride made from totally | — | — | — | 2.00 |
| | hydrogenated palm oil (90% Mono | | | | |
| | Glyceride) + Distilled Water (D) | | | | |
| | Total | 100.00 | 100.00 | 100.00 | 100.00 |

Procedure for BMPX: BMPX comprising Xylitol-90 and bonding agent (A, B, C, D) were manufactured using the procedure A & B similar to Example 1.

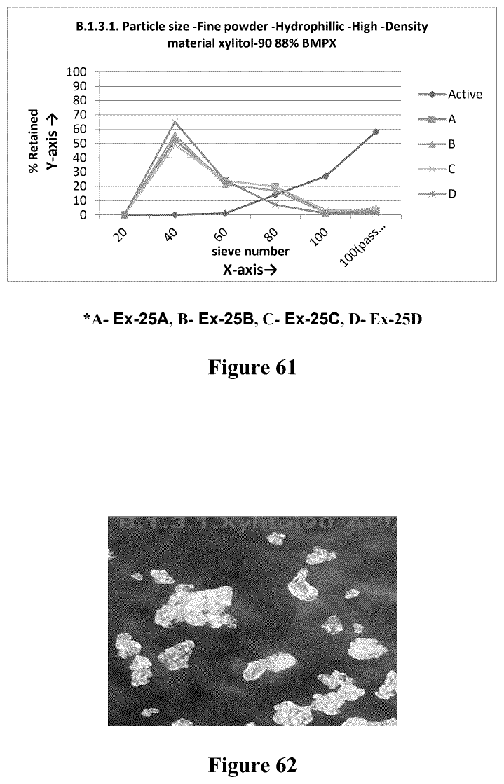

Table 25-B shows Sieve Analysis, bulk density and tapped density of the BMPX of Xylitol-90. FIG. 61 shows comparison of particle size by sieve analysis for BMPX of Xylitol-90 with Bonding Agents A, B, C, D.

TABLE 25-B
Sieve Analysis of Active and BMPX of Xylitol-90.

| | | | Ex-25A | Ex-25B | Ex-25C | Ex-25D |
|---|---|---|---|---|---|---|
| Sieve number | | | Bonding | Bonding | Bonding | Bonding |
| (microns) | | Active | Agent A | Agent B | Agent C | Agent D |
| 20 | (850) | 0 | 0 | 0 | 0 | 0 |
| 40 | (400) | 0 | 52 | 56 | 49 | 65 |
| 60 | (250) | 0 | 24 | 21 | 24 | 24 |
| 80 | (177) | 18 | 20 | 17 | 20 | 7 |
| 100 | (149) | 31.5 | 1 | 1.5 | 3 | 1 |

| | | | | | |
|---|---|---|---|---|---|
| 100 passed | 48.5 | 3 | 4.5 | 4 | 1 |
| Bulk | 0.6 | 0.62 | 0.55 | 0.5 | 0.53 |
| Density(g/ml) | | | | | |
| Tapped | 0.8 | 0.80 | 0.62 | 0.58 | 0.65 |
| Density(g/ml) | | | | | |

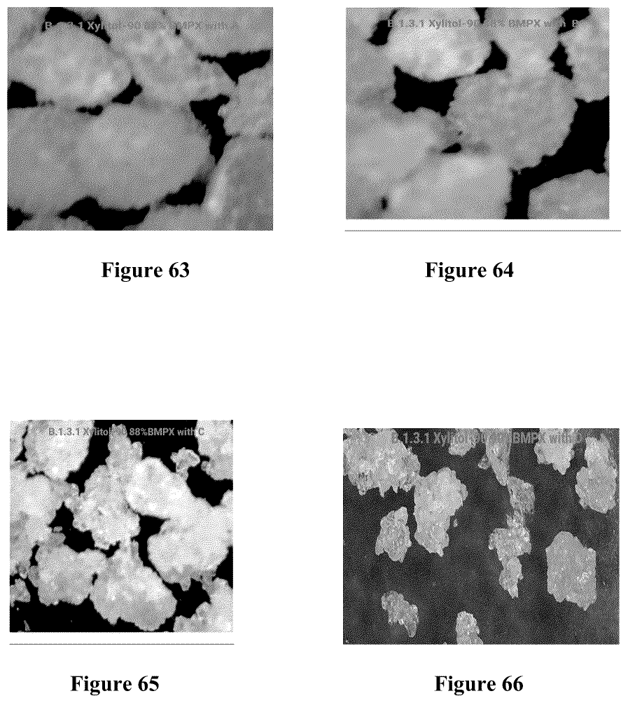

Microscopic Images: FIG. 62 shows the microscopic image of Fine Powder Xylitol-90. FIG. 63 shows the microscopic image of BMPX comprising Fine Powder Xylitol-90 and Bonding Agent A. FIG. 64 shows the microscopic image of BMPX comprising Fine Powder Xylitol-90 and Bonding Agent B. FIG. 65 shows the microscopic image of BMPX comprising Fine Powder Xylitol-90 and Bonding Agent C. FIG. 66 shows the microscopic image of BMPX comprising Fine Powder Xylitol-90 and Bonding Agent D

### Example 26 Dosage Form-Tablet Containing 400 mg of BMPX Xylitol-90

Tablet composition was prepared according to the components and amounts shown in Table-26-A

TABLE 26-A
Tablet containing 400 mg of BMPX Xylitol-90

| | | EX-26A | | EX-26B | | EX-26C | | EX-26D | |
|---|---|---|---|---|---|---|---|---|---|
| | | % | Qty/Dose | % | Qty/Dose | % | Qty/Dose | % | Qty/Dose |
| Components | Function | w/w | (400 mg) | w/w | (400 mg) | w/w | (400 mg) | w/w | (400 mg) |
| BMPX of Xylitol-90 | Active | 97.00 | 454.54 | — | — | — | — | — | — |
| (Ex-25A) | | | | | | | | | |
| BMPX of Xylitol-90 | Active | — | — | 92.00 | 454.50 | — | — | — | — |
| (Ex-25B) | | | | | | | | | |
| BMPX of Xylitol-90 | Active | — | — | — | — | 92.00 | 454.50 | — | — |
| (Ex-25° C.) | | | | | | | | | |
| BMPX of Xylitol-90 | Active | — | — | — | — | — | — | 93.5 | 444.12 |
| (Ex-25D) | | | | | | | | | |
| Micro Crystalline | Diluent | — | — | 5.00 | 24.70 | 5.00 | 24.70 | 5.00 | 23.75 |
| Cellulose | | | | | | | | | |
| Cool mint | Flavor | — | — | 1.50 | 7.41 | — | — | — | — |
| Capsil Grape | Flavor | 1.50 | 7.03 | — | — | — | — | — | — |
| Mango Sd | Flavor | — | — | — | — | 1.50 | 4.94 | — | — |
| Colloidal Silicon | | 0.50 | 7.03 | 0.50 | 2.47 | 0.50 | 2.47 | — | — |
| dioxide | | | | | | | | | |
| Calcium Silicate | Glidant | — | — | — | — | — | — | 0.5 | 2.375 |
| Magnesium | Lubricant | 1.00 | 2.34 | 1.00 | 4.97 | 1.00 | 7.41 | 1.00 | 4.75 |
| Stearate | | | | | | | | | |
| | Total | 100 | 468.60 | 100 | 494.02 | 100 | 494.02 | 100 | 475 |

The BMPX of Xylitol-90 was blended with above ingredients and the blend was compressed into tablet.

Table-26-B shows the characteristics of compressed tablets

TABLE 26-B
Characteristics of compressed tablets

| | Punch | | Compressibility | | | | | | | | | |
|---|---|---|---|---|---|---|---|---|---|---|---|---|
| | shape | Tablet | | Picking | Average | | Compactability | | | | | Disintegration |
| | Punch | Weight | | and | Weight | Weight | Tablet | Tablet | | Tablet | | Time |
| Example | size | (mg) | Flow | sticking | (mg) | Variation | friability | hardness | | thickness | | (Minutes) |
| Ex-26A | Round | 468.60 | Flow | No picking | 466.35 | Within | 0.4% | 7.3 | kp | 3.87 | mm | 4 mins. 3 sec |
| | Lozenge | | is | and | | the limits | | | | | | |
| | shape | | good | sticking is | | | | | | | | |
| | 11 mm | | | observed | | | | | | | | |
| Ex-26B | Round | 494.02 | Flow | No picking | 466.35 | Within | 0.6% | 3.2 | kp | 4.10 | mm | 17 Mins. 25 sec |
| | Lozenge | | is | and | | the limits | | | | | | |
| | shape | | good | sticking is | | | | | | | | |
| | 11 mm | | | observed | | | | | | | | |
| Ex-26C | Round | 494.02 | Flow | No picking | 498.9 | Within | 0.4% | 3.5 | kp | 4.05 | mm | 37 Mins. 14 sec |
| | Lozenge | | is | and | | the limits | | | | | | |
| | shape | | good | sticking is | | | | | | | | |
| | 11 mm | | | observed | | | | | | | | |
| Ex-26D | Round | 475.00 | Flow | No picking | 474.7 | Within | 0.4% | 3.0 | kp | 3.98 | mm | 11 Mins. 37 sec |
| | Lozenge | | is | and | | the limits | | | | | | |
| | shape | | good | sticking is | | | | | | | | |
| | 11 mm | | | observed | | | | | | | | |

Moisture Content: Moisture content was determined for Active, Xylitol-90 BMPX and BMPX Xylitol-90 400 mg tablets by using the Infra Red Moisture Balance. The Moisture content data are shown in Table-26-C.

TABLE 26-C
Moisture content

| Example | Moisture Content |
|---|---|
| Active | 0.1% |
| Ex-25A | 0.1% |
| Ex-25B | 0.1% |
| Ex-25C | 0.1% |
| Ex-26A | 0.1% |
| Ex-26B | 0.1% |
| Ex-26C | 0.1% |

Result: No change of Moisture content in BMPX Xylitol-90 and BMPX Xylitol-90 400 mg tablets was observed when compared to Xylitol-90 active ingredient.

### Example 27: BMPX of Sugar/Sugar Powder

Sugar powder is a crystalline, hydrophilic, high density solid particulate material

Sugar powder BMPX were prepared according to the components and amounts shown in Table-27-A.

TABLE 27-A
BMPX of Sugar/Sugar Powder

| | | Ex-27A | Ex-27B | Ex-27C | Ex-27D |
|---|---|---|---|---|---|
| Components | | Quantity (% w/w) | | | |
| Solid | Pharmaceutically active ingredient | 88.00 | 88.00 | 88.00 | 90.00 |
| particulate | Sugar Powder | | | | |
| material | Form - white micro crystalline powder | | | | |
| | Density -0.73 (High Density) | | | | |
| | Morphology - Crystalline | | | | |
| | Solubility- soluble in water | | | | |
| | Particle size- 149 microns | | | | |
| | Therapeutic Category -Sugar | | | | |
| Bonding agent | Polymer (A) - Polyethylene glycol | 12.00 | — | — | — |
| | 800- (PEG 8000) | | | | |
| | Form - white to off-white powder | | | | |
| | Melting point: 65° C. | | | | |
| | Boiling Point: 250° C. | | | | |
| | Density: 1.125 g/cm3 | | | | |
| | Emulsifier (B) - Monoglyceride made | — | 12.00 | — | 8.00 |
| | from totally hydrogenated palm oil | | | | |
| | (90% Mon- Glyceride) | | | | |
| | Form - beads | | | | |
| | Melting point: 65° C. | | | | |
| | Fat (C)- Hydrogenated soya bean oil | — | — | 12.00 | — |
| | Form - white powder | | | | |
| | Melting point: 66.8° C. | | | | |
| | Monoglyceride made from totally | — | — | — | 2.00 |
| | hydrogenated palm oil (90% Mono | | | | |
| | Glyceride) + | | | | |
| | Distilled Water (D) | | | | |
| | Total | 100.00 | 100.00 | 100.00 | 100.00 |

Procedure for BMPX: BMPX comprising Sugar Powder and bonding agent (A, B, C, D) were manufactured using the Procedure A& B similar to Example 1.

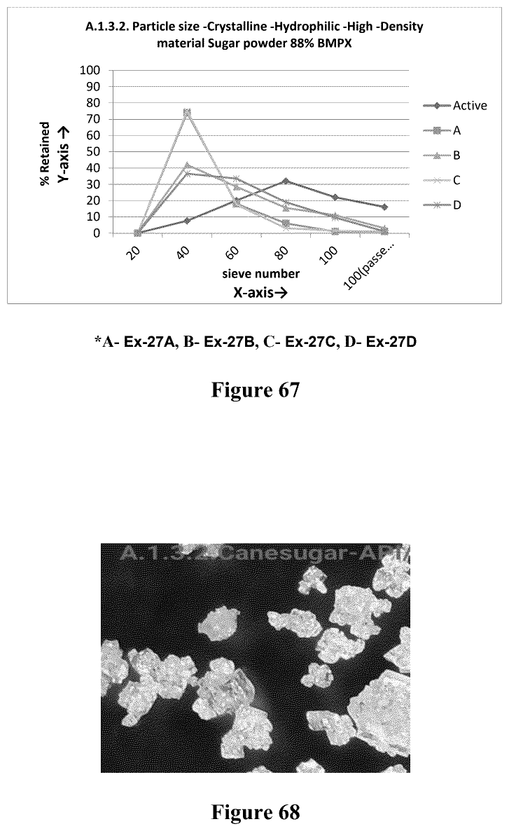

Table 27-B shows Sieve Analysis, bulk density and tapped density of the BMPX of Sugar Powder. FIG. 67 shows comparison of particle size by sieve analysis for BMPX of Sugar powder with Bonding Agents A, B, C, D.

TABLE 27-B
Sieve Analysis of Active and BMPX of Sugar Powder

| | | Ex-27A | Ex-27B | Ex-27C | Ex-27D |
|---|---|---|---|---|---|
| Sieve number | | Bonding Agent | Bonding Agent | Bonding Agent | Bonding |
| (microns) | Active | A | B | C | Agent D |
| 20(850) | 0 | 0 | 0 | 0 | 0 |
| 40 (400) | 7.5 | 74 | 42 | 75 | 36.5 |
| 60 (250) | 20 | 18 | 28.25 | 17.5 | 33.5 |
| 80 (177) | 26 | 6 | 15.5 | 5 | 19 |
| 100 (149) | 22 | 1 | 11 | 1.5 | 9.5 |
| 100 passed | 16 | 1 | 3 | 1 | 1 |
| Bulk | 0.71 | 0.48 | 0.7 | 0.47 | 0.64 |
| Density(g/ml) | | | | | |
| Tapped | 0.73 | 0.55 | 0.75 | 0.55 | 0.73 |
| Density(g/ml) | | | | | |

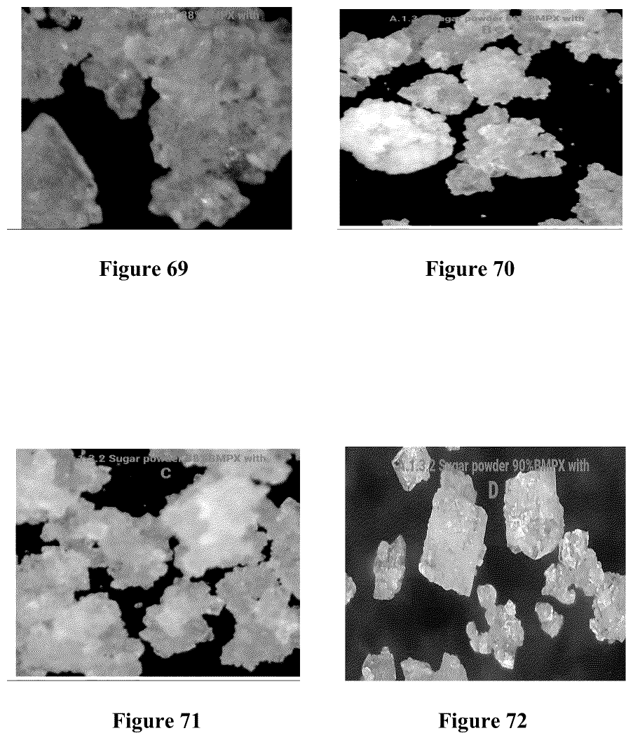

Microscopic Images: FIG. 68 shows the microscopic image of Crystalline Sugar Powder. FIG. 69 shows the microscopic image of BMPX comprising Crystalline Sugar Powder and Bonding Agent A. FIG. 70 shows the microscopic image of BMPX comprising Crystalline Sugar Powder and Bonding Agent B. FIG. 71 shows the microscopic image of BMPX comprising Crystalline Sugar Powder and Bonding Agent C. FIG. 72 shows the microscopic image of BMPX comprising Crystalline Sugar Powder and Bonding Agent D

Example 28 Dosage form-Tablet containing 400 mg of BMPX Sugar powder Tablet composition was prepared according to the components and amounts shown in Table-28-A

TABLE 28-A
Tablet containing 400 mg of BMPX Sugar powder

| | | EX-28A | | EX-28B | | EX-28C | | EX-28D | |
|---|---|---|---|---|---|---|---|---|---|
| | | % | Qty/Dose | % | Qty/Dose | % | Qty/Dose | % | Qty/Dose |
| Components | Function | w/w | (400 mg) | w/w | (400 mg) | w/w | (400 mg) | w/w | (400 mg) |
| BMPX of | Active | 97.00 | 454.54 | — | — | — | — | — | — |
| Sugar Powder | | | | | | | | | |
| (Ex-27A) | | | | | | | | | |
| BMPX of | Active | — | — | 97.00 | 454.54 | — | — | — | — |
| Sugar Powder | | | | | | | | | |
| (Ex-27B) | | | | | | | | | |
| BMPX of | Active | — | — | — | — | 97.00 | 454.54 | — | — |
| Sugar Powder | | | | | | | | | |
| (Ex-27C) | | | | | | | | | |
| BMPX of | Active | — | — | — | — | — | — | 98.50 | 444.432 |
| Sugar Powder | | | | | | | | | |
| (Ex-27D) | | | | | | | | | |
| Magnesium | Lubricant | 1.00 | 4.69 | 1.00 | 4.69 | 1.00 | 4.69 | 1.00 | 4.512 |
| stearate | | | | | | | | | |
| Calcium | Glidant | 0.50 | 2.34 | 0.50 | 2.34 | 0.50 | 2.34 | 0.5 | 2.256 |
| silicate | | | | | | | | | |
| Cool Mint | Flavor | 1.50 | 7.03 | 1.50 | 7.03 | 1.50 | 7.03 | — | — |
| | Total | 100 | 468.60 | 100 | 468.60 | 100 | 468.60 | 100 | 451.20 |

The BMPX of Sugar powder was blended with above ingredients and the blend was compressed into tablet.

Table-28-B shows the characteristics of compressed tablets

TABLE 28-B
Characteristics of compressed tablets

| | Punch | | Compressibility | | | | | | | | | |
|---|---|---|---|---|---|---|---|---|---|---|---|---|
| | shape | Tablet | | Picking | Average | | Compactability | | | | | Disintegration |
| | Punch | Weight | | and | Weight | Weight | Tablet | Tablet | | Tablet | | Time |
| Example | size | (mg) | Flow | sticking | (mg) | Variation | friability | hardness | | thickness | | (Minutes) |
| Ex-28A | Round | 468.6 | Flow | No picking | 464.85 | Within | 0.2% | 8 | kp | 3.80 | mm | 4 mins. 53 sec |
| | Lozenge | | is | and | | the limits | | | | | | |
| | shape | | good | sticking is | | | | | | | | |
| | 11 mm | | | observed | | | | | | | | |
| Ex-28B | Round | 468.6 | Flow | No picking | 466.6 | Within | 0.2% | 4.4 | kp | 3.80 | mm | 24 Mins. 35 sec |
| | Lozenge | | is | and | | the limits | | | | | | |
| | shape | | good | sticking is | | | | | | | | |
| | 11 mm | | | observed | | | | | | | | |
| Ex-28C | Round | 468.6 | Flow | No picking | 464.4 | Within | 0.2% | 5.6 | kp | 3.87 | mm | 42 Mins. 07 sec |
| | Lozenge | | is | and | | the limits | | | | | | |
| | shape | | good | sticking is | | | | | | | | |
| | 11 mm | | | observed | | | | | | | | |
| Ex-28D | Round | 451.2 | Flow | No picking | 451.2 | Within | 0.3% | 4.8 | kp | 3.58 | mm | 15 Mins. 58 sec |
| | Lozenge | | is | and | | the limits | | | | | | |
| | shape | | good | sticking is | | | | | | | | |
| | 11 mm | | | observed | | | | | | | | |

*Disintegration time of Ex28C could be modified by changing the quantity of excipients.

Moisture Content: Moisture content was determined for Active, Sugar powder BMPX and BMPX Sugar powder 400 mg tablets by using the Infra Red Moisture Balance. The Moisture content data are shown in Table-28-C.

TABLE 28-C
Moisture content

| Example | Moisture Content |
|---|---|
| Active | 0.05% |
| Ex-27A | 0.05% |
| Ex-27B | 0.1% |
| Ex-27C | 0.05% |
| Ex-28A | 0.05% |
| Ex-28B | 0.05% |
| Ex-28C | 0.05% |

Result: No significant change of Moisture content in BMPX Sugar powder and BMPX Sugar powder 400 mg tablets was observed when compared to Sugar powder active ingredient.

### Example 29: BMPX of Sugar Free Mannitol

Mannitol is a fine Powder, hydrophobic, medium density solid particulate material

Mannitol BMPX were prepared according to the components and amounts shown in Table-29-A.

TABLE 29-A
BMPX of Sugar Free Mannitol

| | | Ex-29A | Ex-29B | Ex-29C | Ex-29D |
|---|---|---|---|---|---|
| Components | | Quantity (% w/w) | | | |
| Solid particulate | Pharmaceutically active ingredient | 88.00 | 88.00 | 88.00 | 90.00 |
| material | Mannitol | | | | |
| | Form - Fine powder | | | | |
| | Density -0.6(Medium Density) | | | | |
| | Morphology -Fine Powder | | | | |
| | Solubility- insoluble in water | | | | |
| | Particle size- 40 microns | | | | |
| | Therapeutic Category -Sugar free | | | | |
| Bonding agent | Polymer (A) - Polyethylene glycol 800- (PEG | 12.00 | — | — | — |
| | 8000) | | | | |
| | Form - white to off-white powder | | | | |
| | Melting point: 65° C. | | | | |
| | Boiling Point: 250° C. | | | | |
| | Density: 1.125 g/cm3 | | | | |
| | Emulsifier (B) - Monoglyceride made from | — | 12.00 | — | 8.00 |
| | totally hydrogenated palm oil (90% Mon- | | | | |
| | Glyceride) | | | | |
| | Form - beads | | | | |
| | Melting point: 65° C. | | | | |
| | Fat (C)- Hydrogenated soya bean oil | — | — | 12.00 | — |
| | Form - white powder | | | | |
| | Melting point: 66.8° C. | | | | |
| | Monoglyceride made from totally | — | — | — | 2.00 |
| | hydrogenated palm oil (90% Mono Glyceride + | | | | |
| | Distilled Water (D) | | | | |
| | Total | 100.00 | 100.00 | 100.00 | 100.00 |

Procedure for BMPX: BMPX comprising Mannitol and bonding agent (A, B, C) were manufactured using the procedure similar to Example 3.

For process of BMPX with bonding Agent D we use the Procedure B similar to the Example 1

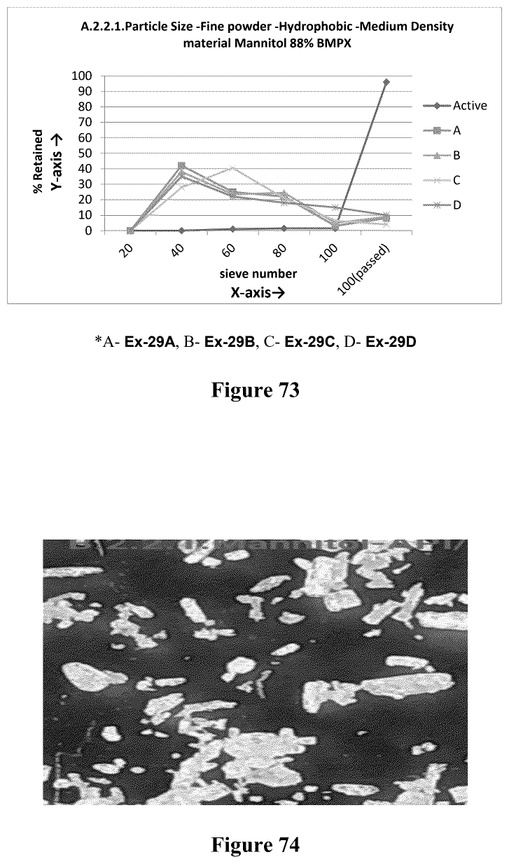

Table 29-B shows Sieve Analysis, bulk density and tapped density of the BMPX of Mannitol. FIG. 73 shows comparison of particle size by sieve analysis for BMPX of Mannitol with Bonding Agents A, B, C, D.

TABLE 29-B
Sieve Analysis of Active and BMPX of Mannitol

| | | Ex-29A | Ex-29B | Ex-29C | Ex-29D |
|---|---|---|---|---|---|
| Sieve number | | Bonding Agent | Bonding Agent | Bonding Agent | Bonding |
| (microns) | Active | A | B | C | Agent D |
| 20(850) | 0 | 0 | 0 | 0 | 0 |
| 40 (400) | 0 | 32 | 38 | 28 | 35 |
| 60 (250) | 1 | 21.5 | 20 | 40.5 | 22 |
| 80 (177) | 1.5 | 22.5 | 23.5 | 21 | 18 |
| 100 (149) | 1.5 | 15 | 12 | 6.5 | 15 |
| 100 passed | 96 | 5 | 6 | 4 | 10 |
| Bulk | 0.5 | 0.43 | 0.62 | 0.47 | 0.45 |
| Density(g/ml) | | | | | |
| Tapped | 0.69 | 0.49 | 0.64 | 0.48 | 0.53 |
| Density(g/ml) | | | | | |

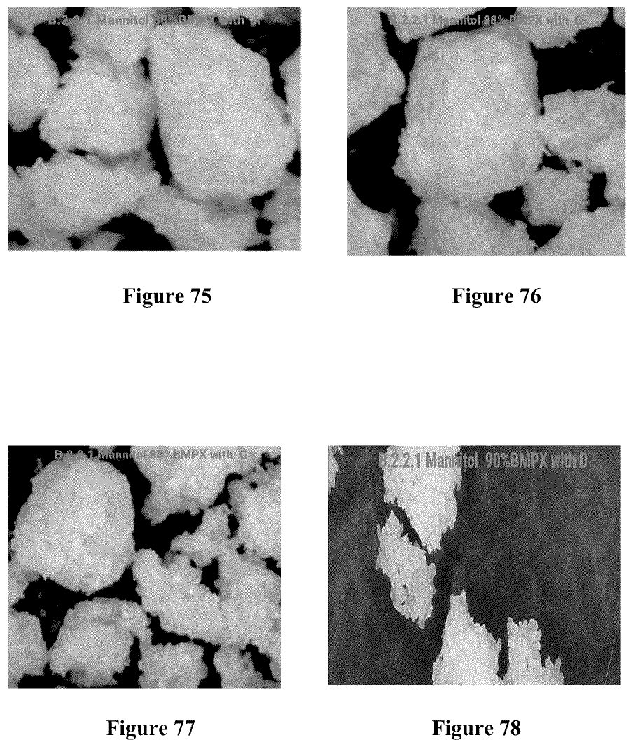

Microscopic Images: FIG. 74 shows the microscopic image of Fine Powder Mannitol. FIG. 75 shows the microscopic image of BMPX comprising Fine Powder Mannitol and Bonding Agent A. FIG. 76 shows the microscopic image of BMPX comprising Fine Powder Mannitol and Bonding Agent B. FIG. 77 shows the microscopic image of BMPX comprising Fine Powder Mannitol and Bonding Agent C. FIG. 78 shows the microscopic image of BMPX comprising Fine Powder Mannitol and Bonding Agent D.

### Example 30 Dosage Form-Tablet Containing 400 mg of BMPX Mannitol

Tablet composition was prepared according to the components and amounts shown in Table-30-A

TABLE 30-A
Tablet containing 400 mg of BMPX Mannitol

| | | EX-30A | | EX-30B | | EX-30C | | EX-30D | | EX-30E | |
|---|---|---|---|---|---|---|---|---|---|---|---|
| | | % | Qty/Dose | % | Qty/Dose | % | Qty/Dose | % | Qty/Dose | % | Qty/Dose |
| Components | Function | w/w | (400 mg) | w/w | (400 mg) | w/w | (400 mg) | w/w | (400 mg) | w/w | (400 mg) |
| BMPX of | Active | 94.25 | 454.50 | — | — | — | — | — | — | — | — |
| Mannitol | | | | | | | | | | | |
| (Ex-29A) | | | | | | | | | | | |
| BMPX of | Active | — | — | 94.25 | 454.50 | — | — | — | — | 99.00 | 454.41 |
| Mannitol | | | | | | | | | | | |
| (Ex-29B) | | | | | | | | | | | |
| BMPX of | Active | — | — | — | — | 94.25 | 470.50 | — | — | — | — |
| Mannitol | | | | | | | | | | | |
| (Ex-29C) | | | | | | | | | | | |
| BMPX of | Active | — | — | — | — | — | — | 94.25 | 444.39 | — | — |
| Mannitol | | | | | | | | | | | |
| (Ex-29D) | | | | | | | | | | | |
| Micro | Diluent | 5.00 | 24.11 | 5.00 | 24.11 | 5.00 | 24.96 | 5.00 | 23.58 | — | — |
| Crystalline | | | | | | | | | | | |
| Cellulose | | | | | | | | | | | |
| Magnesium | Lubricant | 0.75 | 3.62 | 0.75 | 3.62 | 0.75 | 3.14 | 0.75 | 3.54 | 1.00 | 4.59 |
| Stearate | | | | | | | | | | | |
| | Total | 100 | 482.22 | 100 | 482.22 | 100 | 499.2 | 100 | 471.50 | 100 | 459.0 |

The BMPX of Mannitol was blended with above ingredients and the blend was compressed into tablet.

Table-30-B shows the characteristics of compressed tablets.

TABLE 30-B
Characteristics of compressed tablets

| | Punch | | Compressibility | | | | | | | | | |
|---|---|---|---|---|---|---|---|---|---|---|---|---|
| | shape | Tablet | | Picking | Average | | Compactability | | | | | Disintegration |
| | Punch | Weight | | and | Weight | Weight | Tablet | Tablet | | Tablet | | Time |
| Example | size | (mg) | Flow | sticking | (mg) | Variation | friability | hardness | | thickness | | (Minutes) |
| Ex-30A | Round | 482.2 | Flow | No picking | 482.35 | Within | 0.2% | 8.9 | kp | 4.09 | mm | 8 Mins. 16 sec |
| | Lozenge | | is | and | | the limits | | | | | | |
| | shape | | good | sticking is | | | | | | | | |
| | 11 mm | | | observed | | | | | | | | |
| Ex-30B | Round | 482.2 | Flow | No picking | 488.85 | Within | 0.2% | 5.4 | kp | 4.10 | mm | 19 Mins. 45 sec |
| | Lozenge | | is | and | | the limits | | | | | | |
| | shape | | good | sticking is | | | | | | | | |
| | 11 mm | | | observed | | | | | | | | |
| Ex-30C | Round | 499.2 | Flow | No picking | 481.8 | Within | 0.4% | 5.8 | kp | 4.21 | mm | 114 Mins. 07 sec |
| | Lozenge | | is | and | | the limits | | | | | | |
| | shape | | good | sticking is | | | | | | | | |
| | 11 mm | | | observed | | | | | | | | |
| Ex-30D | Round | 471.50 | Flow | No picking | 466.8 | Within | 0.4% | 3.9 | kp | 3.99 | mm | 15 Mins. 45 sec |
| | Lozenge | | is | and | | the limits | | | | | | |
| | shape | | good | sticking is | | | | | | | | |
| | 11 mm | | | observed | | | | | | | | |
| Ex-30E | Round | 459.0 | Flow | No picking | 459.5 | Within | 0.5% | 5.4 | kp | 3.94 | mm | 25 Mins. 39 sec |
| | Lozenge | | is | and | | the limits | | | | | | |
| | shape | | good | sticking is | | | | | | | | |
| | 11 mm | | | observed | | | | | | | | |

*Disintegration time of Ex30C could be modified by changing the quantity of excipients.

Moisture Content: Moisture content was determined for Active, Mannitol BMPX and BMPX Mannitol 400 mg tablets by using the Infra Red Moisture Balance. The Moisture content data are shown in Table-30-C.

TABLE 30-C
Moisture content

| Example | Moisture Content |
|---|---|
| Active | 0.1% |
| Ex-29A | 0.05% |
| Ex-29B | 0.1% |
| Ex-29C | 0.05% |
| Ex-30A | 0.05% |
| Ex-30B | 0.05% |
| Ex-30C | 0.05% |
| Ex-30E | 0.05% |

Result: No significant change in Moisture content of BMPX Mannitol and BMPX Mannitol 400 mg tablets was observed when compared to Mannitol active ingredient.

### Example 31: BMPX of Calcium Supplement Dibasic Calcium Phosphate Anhydrous

Dibasic Calcium phosphate Anhydrous is a fine powder, hydrophobic, high Density solid particulate material

Dibasic Calcium Phosphate Anhydrous BMPX were prepared according to the components and amounts shown in Table-31-A.

TABLE 31-A
BMPX of Calcium Supplement Dibasic Calcium Phosphate Anhydrous

| | | Ex-31A |
|---|---|---|
| | | Quantity |
| Components | | (% w/w) |
| Solid particulate | Pharmaceutically active ingredient | 88.00 |
| material | Dibasic Calcium Phosphate Anhydrous | |
| | Form - white powder | |
| | Density- 0.86(Medium density) | |
| | Morphology -Fine Powder | |
| | Solubility- in soluble in water | |
| | Particle size- 149 microns | |
| | Melting point -109° C. | |
| | Therapeutic Category - Calcium Supplement | |
| | Emulsifier(A) - Monoglyceride made from totally | 12.00 |
| | hydrogenated palm oil (90% Mono Glyceride) | |
| | Form - beads | |
| | Melting point: 65° C. | |
| | Total | 100.00 |

Procedure for BMPX: BMPX comprising Dibasic Calcium Phosphate Anhydrous and bonding agent (B) were manufactured using the procedure similar to Example 3.

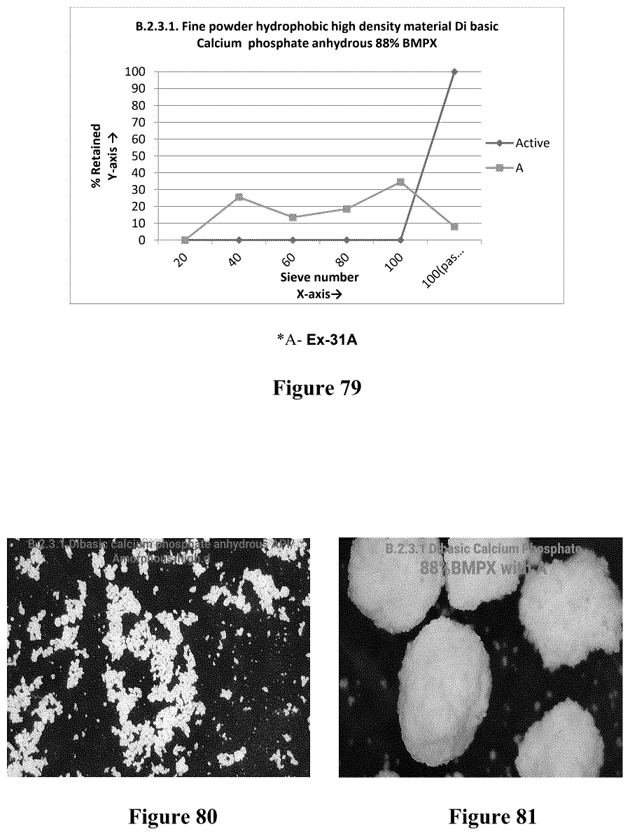

Table 31-B shows Sieve Analysis, bulk density and tapped density of the BMPX of Dibasic Calcium Phosphate Anhydrous. FIG. 79 shows comparison of particle size by sieve analysis for BMPX of Dibasic Calcium Phosphate Anhydrous with Bonding Agents A.

TABLE 31-B
Sieve Analysis of Active and BMPX of
Dibasic Calcium Phosphate Anhydrous

| Sieve number | | Ex-31A |
|---|---|---|
| (Microns) | Active | Bonding Agent A |
| 20(850) | 0 | 0 |
| 40 (400) | 0 | 25.5 |
| 60 (250) | 0 | 13.5 |
| 80 (177) | 0 | 18.5 |
| 100 (149) | 20 | 34.5 |
| 100 passed | 100 | 8 |
| Bulk Density (g/ml) | 1.0 | 0.65 |
| Tapped density (g/ml) | 1.1 | 0.75 |

Microscopic Images: FIG. 80 shows the microscopic image of Fine Powder Dibasic Calcium phosphate Anhydrous. FIG. 81 shows the microscopic image of BMPX comprising Fine Powder Dibasic Calcium phosphate anhydrous and Bonding Agent A.

### Example 32 Dosage Form-Tablet Containing 400 mg of BMPX Dibasic Calcium Phosphate Anhydrous

Tablet composition was prepared according to the components and amounts shown in Table-32-A

TABLE 32-A
Tablet containing 400 mg of BMPX Dibasic
Calcium phosphate Anhydrous

| | | EX-32A | |
|---|---|---|---|
| | | | Qty/Dose |
| Components | Function | % w/w | (400 mg) |
| BMPX of Dibasic Calcium | Active | 99.00 | 454.41 |
| Phosphate Anhydrous (Ex-31A) | | | |
| Magnesium stearate | Lubricant | 1.00 | 4.59 |
| | Total | 100 | 459.0 |

The above BMPX of Dibasic calcium phosphate were blended with Above ingredients and the blend was compressed into tablet.

Table-32-B shows the characteristics of compressed tablets.

TABLE 32-B
Characteristics of compressed tablets

| | Punch | | Compressibility | | | | | | | |
|---|---|---|---|---|---|---|---|---|---|---|
| | shape | Tablet | | Picking | Average | | Compactability | | | Disintegration |
| | Punch | Weight | | and | Weight | Weight | Tablet | Tablet | Tablet | time |
| Example | size | (mg) | Flow | sticking | (mg) | Variation | friability | hardness | thickness | (Minutes)s |
| Ex-32A | Round | 459.0 | Flow | No picking | 458.9 | Within | 0.1% | 10 kp | 3.68 mm | 120 mins. 10 sec |
| | Lozenge | | is | and | | the limits | | | | |
| | shape | | good | sticking is | | | | | | |
| | 9 mm | | | observed | | | | | | |

*Disintegration time of Ex32A could be modified by changing the quantity of excipients.

Moisture Content: Moisture content was determined for Active, Dibasic Calcium Phosphate Anhydrous BMPX and BMPX Dibasic Calcium Phosphate Anhydrous 400 mg tablets by using the Infra Red Moisture Balance. The Moisture content data are shown in Table-32-C.

TABLE 32-C
Moisture content

| Example | Moisture Content |
|---|---|
| Active | 0.1% |
| Ex-31A | 0.1% |
| Ex-32A | 0.1% |

Result: No change in Moisture content of BMPX Dibasic Calcium Phosphate Anhydrous and BMPX Dibasic Calcium Phosphate Anhydrous 400 mg tablets was observed, when compared to Dibasic Calcium Phosphate Anhydrous active ingredient.

### Example 33: BMPX of Sweetener/Diluent Lactose Anhydrous

Lactose Anhydrous is a fine powder, hydrophobic, Medium Density solid particulate material

Lactose Anhydrous BMPX were prepared according to the components and amounts shown in Table-33-A.

TABLE 33-A
BMPX of Sweetener/Diluent Lactose Anhydrous

| Components | | |
|---|---|---|
| Solid particulate | Pharmaceutically active | Ex-33A |
| material | ingredient | Quantity (% w/w) |
| | Lactose Anhydrous | 88.00 |
| | Form - white powder | |
| | Density- 0.75(Medium density) | |
| | Morphology -Fine Powder | |
| | Solubility- in soluble in water | |
| | Particle size- 149 microns | |
| | Melting point -202.8° C. | |
| | Therapeutic Category - | |
| | Sweetener | |
| | Emulsifier(A) - Monoglyceride | 12.00 |
| | made from totally hydrogenated | |
| | palm oil (90% Mon- Glyceride) | |
| | Form - beads | |
| | Melting point: 65° C. | |
| | Total | 100.00 |

Procedure for BMPX: BMPX comprising Lactose Anhydrous and bonding agent (A) were manufactured using the process similar to Example 3.

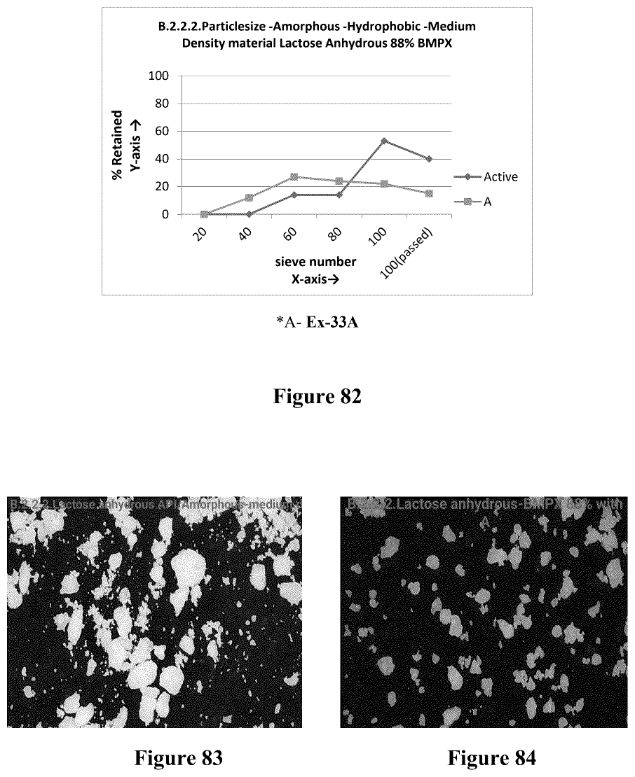

Table 33-B shows Sieve Analysis, bulk density and tapped density of the BMPX of Lactose Anhydrous. FIG. 82 shows comparison of particle size by sieve analysis for BMPX of Lactose Anhydrous with Bonding Agents A.

TABLE 33-B
Sieve Analysis of Active and BMPX of Lactose Anhydrous

| Sieve number | | Ex-33A |
|---|---|---|
| (Microns) | Active | Bonding Agent A |
| 20(850) | 0 | 0 |
| 40 (400) | 0 | 12 |
| 60 (250) | 14 | 27 |
| 80 (177) | 17 | 24 |
| 100 (149) | 53 | 22 |
| 100 passed | 40 | 25 |
| Bulk Density (g/ml) | 0.55 | 0.5 |
| Tapped density (g/ml) | 0.73 | 0.58 |

Microscopic Images: FIG. 83 shows the microscopic image of Fine Powder Lactose Anhydrous. FIG. 84 shows the microscopic image of BMPX comprising Fine Powder Lactose anhydrous and Bonding Agent A.

### Example 34 Dosage Form-Tablet Containing 500 mg of BMPX Lactose Anhydrous

Tablet composition was prepared according to the components and amounts shown in Table-34-A

TABLE 34-A
Tablet containing 500 mg of BMPX Lactose Anhydrous

| | | EX-34A | | EX-34B | |
|---|---|---|---|---|---|
| | | | Qty/Dose | | Qty/Dose |
| Components | Function | % w/w | (500 mg) | % w/w | (500 mg) |
| BMPX of Lactose | Active | 98.80 | 568.10 | 88.25 | 568.15 |
| Anhydrous (Ex-33A) | | | | | |
| Magnesium stearate | Lubricant | 1.00 | 5.75 | 0.75 | 4.83 |
| Colloidal silicon Dioxide | Glidant | 0.20 | 1.15 | 3.00 | 19.31 |
| Micro crystalline Cellulose | Diluent | — | — | 5.00 | 32.19 |
| Sodium Starch glycolate | Disintegrant | — | — | 3.00 | 19.31 |
| | Total | 100 | 575.00 | 100 | 643.80 |

The above BMPX of Lactose Anhydrous were blended with Above ingredients and the blend was compressed into tablet.

Table-34-B shows the characteristics of compressed tablets.

TABLE 34-B
Characteristics of compressed tablets.

| | Punch | | Compressibility | | | | | | | |
|---|---|---|---|---|---|---|---|---|---|---|
| | shape | Tablet | | Picking | Average | | Compactability | | | Disintegration |
| | Punch | Weight | | and | Weight | Weight | Tablet | Tablet | Tablet | time |
| Example | size | (mg) | Flow | sticking | (mg) | Variation | friability | hardness | thickness | (Minutes)s |
| Ex-32A | Round | 459.0 | Flow | No picking | 458.9 | Within | 0.1% | 10 kp | 3.68 mm | 120 mins. 10 sec |
| | Lozenge | | is | and | | the limits | | | | |
| | shape | | good | sticking is | | | | | | |
| | 9 mm | | | observed | | | | | | |

Moisture Content: Moisture content was determined for Active, Lactose Anhydrous BMPX and BMPX Lactose Anhydrous 500 mg tablets by using the Infra Red Moisture Balance. The Moisture content data are shown in Table-34-C.

TABLE 34-C
Moisture content

| Example | Moisture Content |
|---|---|
| Active | 0.2% |
| Ex-33A | 0.2% |
| Ex-34A | 0.2% |
| Ex-34B | 0.2% |

Result: No change in Moisture content of BMPX Lactose Anhydrous and BMPX Lactose Anhydrous 500 mg tablets was observed, when compared to Lactose Anhydrous active ingredient.

### Example Embodiments

The following examples pertain to specific technology embodiments and point out specific features, elements, or steps that may be used or otherwise combined in achieving such embodiments.

In one example there is provided compositions, processes and applications of high percentage solid particulate materials comprising (a) high percentage solid particulate material by weight, based on a total weight of the bonded microparticulates, selected from the group of pharmaceutically active ingredient, a bioactive agent, oral care agent, a dietary supplement, a pharmaceutical excipient, a food ingredient, an agrochemical, and an animal food having different forms as amorphous, crystalline or combination thereof, having different solubilities and different particle densities; (b) low percentage of Bonding agents by weight, based on a total weight of the bonded microparticulates, selected from the group of polymers, emulsifier, fat and combinations thereof; and (c) to form bonded microparticles, which can further be used to manufacture different dosage forms.

In one example of compositions, processes and applications of high percentage solid particulate materials, the solid particulate material with different solubilities include hydrophilic material, hydrophobic material and lipophilic material.

In one example of compositions, processes and applications of high percentage solid particulate materials, the solid particulate material with different particle density includes low density material, medium density material and high-density material.

In one example of compositions, processes and applications of high percentage solid particulate materials, the high percentage solid particulate material comprises of more than about 75% of solid particulate material by weight based on a total weight of the bonded microparticulates.

In one example of compositions, processes and applications of high percentage solid particulate materials, the high percentage solid particulate material comprises of more than about 80% of solid particulate material by weight based on a total weight of the bonded microparticulates.

In one example of compositions, process and applications of high percentage solid particulate materials, the low percentage bonding agent comprise of not more than about 25% of bonding agent by weight based on a total weight of the bonded microparticulates.

In one example of compositions, processes and applications of high percentage solid particulate materials, the low percentage bonding agent comprise of not more than about 20% of bonding agent by weight based on a total weight of the bonded microparticulates.

In one example of compositions, processes and applications of high percentage solid particulate materials, the pharmaceutically active ingredient is selected from the group consisting of an antidiabetic drug, a therapeutic drug for diabetic complications, a lipid lowering agent, an antihypertensive drug, an antiobesity drug, an anti-clotting agent, an anticoagulant drug, an opioid for pain management, an analgesic, an anti-inflammatory agent, an antihistamine, a steroid drug, a bronchodilator, an anticholinergic agent, an antibiotic drug, an anti-fungal drug, an antiviral drug, an anti-emetic, a leukotriene receptor antagonist, a sympathomimetic drug, an acetylcholinesterase inhibitor, an immunomodulator, a phosphodiesterase inhibitor, an antidepressant, a serotonin agonist, a serotonin antagonist, an adrenergic agonist, an adrenergic antagonist, an adrenergic neuron blocker, a benzodiazepine, an anticonvulsant, a calcium channel blocker, an antiarrhythmic, a potassium channel modulator, a diuretic, a smoking cessation drug, a bisphosphonate, a dopamine agonist, a nucleic-acid medicine, an antipsychotic, prebiotic, a central nervous system stimulant, an expectorant, an antacid, a zinc compound used in the treatment of colds, and a combination thereof.

In one example of compositions, processes and applications of high percentage solid particulate materials, the dietary supplements, foods, active ingredient is selected from the group consisting of vitamins, minerals, herbs, amino acids, fatty acids, probiotics, dietary fiber materials, calcium and other mineral supplements, carbohydrates, glycoconjugates, enzymes, metabolites, and combinations thereof.

In one example of compositions, processes and applications of high percentage solid particulate materials, the excipient is selected from the group consisting of sugars, celluloses, polyols, mineral salts, mannitol, xylitol, sorbitol, glucose, sucrose, dextrose, lactose, microcrystalline cellulose, calcium phosphate, dicalcium phosphate, fructose, calcium sulfate, calcium carbonate, magnesium salts, and mixtures thereof.

In one example of compositions, processes and applications of high percentage solid particulate materials, the oral care agents are selected from one or more of fluoride ion sources, pH modifiers, like Sodium bicarbonate or sodium carbonates and or potassium bicarbonate or related salts, anticalculus or anti-tartar agents, antimicrobial agents, anti-dry mouth agents, buffers, abrasives such a silica, alkali metal bicarbonate salts, thickening materials, humectants, water, surfactants, titanium dioxide, flavorants, sweetening agents, coolants and other sensate, xylitol, and coloring agents.

In one example of compositions, processes and applications of high percentage solid particulate materials, the oral care agent is selected from one or more of Xylitol, Sorbitol, Maltitol, Erythritol, Alluolse, Mannnitol or polyols or zinc salts which are non-carcinogenic and tooth friendly.

In one example of compositions, processes and applications of high percentage solid particulate materials, the bonding agent is an agent that softens at temperature of from about 30° C. to about 100° C.

In one example of compositions, processes and applications of high percentage solid particulate materials, the bonding agent is an agent softens at a temperature of about 60° C. or less.

In one example of compositions, processes and applications of high percentage solid particulate materials, the bonding agent is one or more polymers selected from the group consisting of homopolymers and copolymers of N-vinyl lactams, e.g., homopolymers and copolymers of N-vinyl pyrrolidone (e.g., polyvinylpyrrolidone), copolymers of N-vinyl pyrrolidone and vinyl acetate or vinyl propionate; cellulose esters and cellulose ethers (e.g., methylcellulose and ethylcellulose) hydroxyalkylcelluloses (e.g., hydroxypropylcellulose), hydroxyalkylalkylcelluloses (e.g., hydroxypropylmethylcellulose), cellulose phthalates (e.g., cellulose acetate phthalate and hydroxylpropylmethylcellulose phthalate) and cellulose succinates (e.g., hydroxypropylmethylcellulose succinate or hydroxypropylmethylcellulose acetate succinate); high molecular polyalkylene oxides such as polyethylene oxide and polypropylene oxide and copolymers of ethylene oxide and propylene oxide; polyacrylates and polymethacrylates (e.g., methacrylic acid/ethyl acrylate copolymers, methacrylic acid/methyl methacrylate copolymers, butyl methacrylate/2-dimethylaminoethyl methacrylate copolymers, poly(hydroxyalkyl acrylates), poly(hydroxyalkyl methacrylates)); polyacrylamides; vinyl acetate polymers such as copolymers of vinyl acetate and crotonic acid, partially hydrolyzed polyvinyl acetate; and oligo- and polysaccharides, such as carrageenans, galactomannans and xanthan gum, Polyethylene glycol 2000, Polyethylene glycol 3000, Polyethylene glycol 6000, Polyethylene glycol 8000, Polyethylene glycol 10000, Polyethylene glycol 20000 and polyethylene glycol palmitostearate (e.g., Stearate 6000 WL 1644®), Polyvinyl alcohol and acetate, Ethyl Vinayl acetate.

In one example of compositions, process and applications of high percentage solid particulate materials, the polymer is selected from the group consisting of Poloxamer 188, Polyethylene glycol 2000, Polyethylene glycol 3000, Polyethylene glycol 6000, Polyethylene glycol 8000, Polyethylene glycol 10000, Polyethylene glycol 20000, and polyethylene glycol palmitostearate.

In one example of compositions, processes and applications of high percentage solid particulate materials, the bonding agent is one or more emulsifiers selected from polyethoxylated fatty acids such as PEG-8 laurate, PEG-8 oleate, PEG-8 stearate, PEG-9 oleate, PEG-10 laurate, PEG-10 oleate, PEG-12 laurate, PEG-12 oleate, PEG-15 oleate, PEG-20 laurate and PEG-20 oleate; PEG-fatty acid diesters such as PEG-20 dilaurate, PEG-dioleate, PEG-20 distearate, PEG-32 dilaurate and PEG-32 dioleate; PEG-fatty acid mono- and di-ester mixtures; polyethylene glycol glycerol fatty acid esters such as PEG-20 glyceryl laurate, PEG-30 glyceryl laurate, PEG-40 glyceryl laurate, PEG-20 glyceryl oleate, and PEG-glyceryl oleate; alcohol-oil transesterification products such as PEG-35 castor oil (Incrocas-35), PEG-40 hydrogenated castor oil (Cremophor® RH40), polyoxyl 35 castor oil (Cremophor EL), PEG-25 trioleate (TAGAT® TO), PEG-60 corn glycerides (Crovol M70), PEG-60 almond oil (Crovol A70), PEG-40 palm kernel oil (Crovol PK70), PEG-50 castor oil (Emalex C-50), PEG-50 hydrogenated castor oil (Emalex HC-50), PEG-8 caprylic/capric glycerides (Labrasol®), and PEG-6 caprylic/capric glycerides (Softigen® 767); transesterification products of oils and alcohols; polyglycerized fatty acids such as polyglyceryl oleate (Plurol® Oleique), polyglyceryl-2 dioleate (Nikkol DGDO), and polyglyceryl-10 trioleate. Preferred emulsifier includes polyglyceryl-10 laurate (Nikkol Decaglyn 1-L), polyglyceryl-10 oleate (Nikkol Decaglyn 1-0), and polyglyceryl-10 mono, dioleate (Caprol® PEG 860); propylene glycol fatty acid esters such as propylene glycol monolaurate (Lauroglycol FCC), propylene glycol ricinoleate (Propymuls), propylene glycol monooleate (Myverol® P-06), propylene glycol dicaprylate/dicaprate (Captex® 200), and propylene glycol dioctanoate (Captex 800); mixtures of propylene glycol esters and glycerol esters such as a mixture of oleic acid esters of propylene glycol and glycerol (Arlacel 186); mono- and diglycerides such as glyceryl monooleate (Peceol), glyceryl ricinoleate, glyceryl laurate, glyceryl dilaurate (Capmul® GDL), glyceryl dioleate (Capmul GDO), glyceryl mono/dioleate (Capmul GMO-K), glyceryl caprylate/caprate (Capmul MCM), caprylic acid mono/diglycerides (Imwitor® 988), and mono- and diacetylated monoglycerides (Myvacet® 9-45); sterol and sterol derivatives such as PEG-24 cholesterol ether (Solulan® C-24); polyethylene glycol sorbitan fatty acid esters such as PEG-20 sorbitan monolaurate (Tween® 20), PEG-20 sorbitan monopalmitate (Tween 40), PEG-20 sorbitan monostearate (Tween 60), and PEG-20 sorbitan monooleate (polysorbate 80 or Tween 80); polyethylene glycol alkyl ethers such as PEG-3 oleyl ether (Volpo 3) and PEG-4 lauryl ether (Brij 30); sugar esters such as sucrose monopalmitate and sucrose monolaurate; polyethylene glycol alkyl phenols; polyoxyethylene-polyoxypropylene block copolymers such as Synperonic® PE series (ICI); Pluronic® series (BASF), Emkalyx, Lutrol (BASF), Supronic, Monolan, Pluracare®, and Plurodac; sorbitan fatty acid esters such as sorbitan monolaurate (Arlacel® 20), sorbitan monopalmitate (Span-40), sorbitan monooleate (Span-80), sorbitan monostearate, and sorbitan tristearate; lower alcohol fatty acid esters such as hydrophobic surfactants include ethyl oleate (Crodamol EO), isopropyl myristate (Crodamol IPM), and isopropyl palmitate (Crodamol IPP), PEG-400 succinate, PEG 3350, tocopherol polyethyleneglycol (200-8000 MW) succinate, tocopherol polyethylene glycol 400 succinate, tocopherol polyethyleneglycol 1000 succinate (Vitamin E-TPGS, Eastman Chemical Co.), glycerol monolinoleate (Maisine®), propylene glycol monocaprylate (Capryol® 90); caprylocaproyl macrogol-8 glycerides (Labrosol®), glycerol dibehenate (Compritol® 888), glycerol distearate (Precirol®), lauroyl macrogol-32 glycerides (Gelucire® 44/14), and stearoyl macrogol-32 glycerides (Gelucire 50/13).

In one example of compositions, processes and applications of high percentage solid particulate materials, the emulsifier is selected from the group consisting of PEG-20 castor oil, PEG-23 castor oil, PEG-30 castor oil, PEG-40 castor oil, PEG-56 castor oil, PEG-60 castor oil, PEG-100 castor oil, PEG-200 castor oil, PEG-20 hydrogenated castor oil, PEG-25 hydrogenated castor oil, PEG-30 hydrogenated castor oil, PEG-40 hydrogenated castor oil, PEG-45 hydrogenated castor oil, PEG-60 hydrogenated castor oil, PEG-80 hydrogenated castor oil, PEG-100 hydrogenated castor oil, PEG-20 almond glycerides, PEG-25 trioleate, PEG-40 hydrogenated palm oil, PEG-60 corn glycerides, PEG-60 almond glycerides, PEG-8 caprylic/capric glycerides, PEG-6 caprylic/capric glycerides, lauroyl macrogol-32 glyceride, and stearoyl macrogol-32 glyceride.

In one example of compositions, processes and applications of high percentage solid particulate materials, the bonding agent is one or more fats selected from the group fats, triglycerides, hydrogenated natural oil or non-hydrogenated fat-fractionated Palm oils or others similar. In some embodiments, the hydrogenated natural oil is selected from the group consisting of hydrogenated canola oil, hydrogenated rapeseed oil, hydrogenated coconut oil, hydrogenated corn oil, hydrogenated cottonseed oil, hydrogenated olive oil, hydrogenated palm oil, hydrogenated peanut oil, hydrogenated safflower oil, hydrogenated sesame oil, hydrogenated soybean oil, hydrogenated sunflower oil, hydrogenated linseed oil, hydrogenated palm kernel oil, hydrogenated tung oil, hydrogenated jatropha oil, hydrogenated mustard oil, hydrogenated camelina oil, hydrogenated pennycress oil, hydrogenated castor oil, hydrogenated derivatives of these oils, and mixtures thereof.

In one example of compositions, processes and applications of high percentage solid particulate materials, the process of preparing bonded microparticulates comprises a) softening a bonding agent at a temperature between room temperature and the melting point of the bonding agent; b) contacting the solid particulate material with the softened bonding agent to form a mixture comprising the solid particulate material and the bonding agent; c) cooling the mixture to a predetermined temperature to produce bonded microparticulates.

In one example of compositions, processes and applications of high percentage solid particulate materials, the process of preparing bonded microparticulate comprises a) softening a bonding agent at a temperature between room temperature and the melting point of the bonding agent; b) adding water to softened bonding agent and mixing; c) contacting the solid particulate material with the softened bonding agent of step b) to form a mixture comprising the solid particulate material and the bonding agent; and d) cooling the mixture to a predetermined temperature to produce bonded microparticulates.

In one example of compositions, processes and applications of high percentage solid particulate materials, the process does not involve drying and/or milling step.

In one example of compositions, process and applications of high percentage solid particulate materials the bonded microparticulates are compressed into a tablet.

In one example of compositions, processes and applications of high percentage solid particulate materials, bonded microparticulates, prior to compressing are blended with at least one pharmaceutically acceptable excipient.

In one example of compositions, processes and applications of high percentage solid particulate materials, the pharmaceutically acceptable excipient is one or more ingredients selected from diluents, drug release-controlling excipients, binders, disintegrants, lubricants, glidants, surfactants, stabilizers, preservatives, alkalizing agents, anti-adherents, sweeteners, flavoring agents, coloring agents, or mixtures thereof.

In one example of compositions, processes and applications of high percentage solid particulate materials, the dosage form can be tablets, capsules, pills, lozenges, caplets, dry suspensions, rapid-melt tablets, chew tablets, rapid-melt beads, dry injectable compositions, or topical skin care compositions.

In one example of compositions, processes and applications of high percentage solid particulate materials, the solid particulate material is selected from Metformin, Niacin, Guaifenesin, Amlodipine Besylate, Caffeine Anhydrous, Diphenhydramine, Sodium Bicarbonate, Zinc Gluconate, Fructo oligosaccharide, Calcium Carbonate, Xylitol, Sugar/Sugar Powder, Sugar Free Mannitol, Ibuprofen, Dibasic Calcium Phosphate and lactose.

1. A bonded microparticulate (BMPX) composition comprising a combination of a solid particulate material and a solid bonding agent, wherein the bonding agent is present in an amount of no more than about 25 w/w % of the composition.

2. The BMPX composition of claim 1, wherein:
the amount of bonding agent is about 20 w/w % or less; or
the amount of solid particulate material is more than about 75% w/w; or
a combination thereof.

3-6. (canceled)

7. The BMPX composition of claim 1, wherein the bonding agent includes one or more of a polymer, an emulsifier, a fat, or a combination thereof.

8. The BMPX composition of claim 7, wherein the polymer is a single polymer or a combination of polymers.

9. The BMPX composition of claim 1, wherein solid particulate material has a crystalline form, a quasi-crystalline form, or an amorphous form or combination thereof.

10. The BMPX composition of claim 1, wherein the solid particulate material is one or more of a pharmaceutically active ingredient, a bioactive agent, an oral care agent, a dietary supplement, a pharmaceutical excipient, a food ingredient, an agrochemical, an animal food, or a combination thereof.

11. The BMPX composition of claim 10, wherein the solid particulate material is a pharmaceutically active agent selected from the group consisting of:
an antidiabetic drug, a therapeutic drug for diabetic complications, a lipid lowering agent, an antihypertensive drug, an antiobesity drug, an anti-clotting agent, an anticoagulant drug, an opioid for pain management, an analgesic, an anti-inflammatory agent, an antihistamine, a steroid drug, a bronchodilator, an anticholinergic agent, an antibiotic drug, an anti-fungal drug, an antiviral drug, an anti-emetic, a leukotriene receptor antagonist, a sympathomimetic drug, an acetylcholinesterase inhibitor, an immunomodulator, a phosphodiesterase inhibitor, an antidepressant, a serotonin agonist, a serotonin antagonist, an adrenergic agonist, an adrenergic antagonist, an adrenergic neuron blocker, a benzodiazepine, an anticonvulsant, a calcium channel blocker, an antiarrhythmic, a potassium channel modulator, a diuretic, a smoking cessation drug, a bisphosphonate, a dopamine agonist, a nucleic-acid medicine, an antipsychotic, prebiotic, a central nervous system stimulant, an expectorant, an antacid, a zinc compound used in the treatment of colds, and a combination thereof.

12. The BMPX composition of claim 11, wherein the solid particulate material comprises at least one of: Metformin, Niacin, Guaifenesin, Amlodipine Besylate, Caffeine Anhydrous, Diphenhydramine, Sodium Bicarbonate, Zinc Gluconate, Fructo oligosaccharide, Calcium Carbonate, Xylitol, Sugar/Sugar Powder, Sugar Free Mannitol, Ibuprofen, Dibasic Calcium Phosphate and lactose.

13. The BMPX composition of claim 1, wherein the bonding agent is an agent that softens at a temperature of from about 30° C. to about 100° C.

14. (canceled)

15. The BMPX composition of claim 1, wherein the bonding agent is one or more polymers selected from the group consisting of: homopolymers and copolymers of N-vinyl lactams, including homopolymers and copolymers of N-vinyl pyrrolidone including polyvinylpyrrolidone, copolymers of N-vinyl pyrrolidone and vinyl acetate or vinyl propionate; cellulose esters and cellulose ethers including methylcellulose and ethylcellulose, hydroxyalkylcelluloses including hydroxypropylcellulose, hydroxyalkylalkylcelluloses including hydroxypropylmethylcellulose), cellulose phthalates including cellulose acetate phthalate and hydroxylpropylmethylcellulose phthalate and cellulose succinates including hydroxypropylmethylcellulose succinate or hydroxypropylmethylcellulose acetate succinate; high molecular polyalkylene oxides including polyethylene oxide and polypropylene oxide and copolymers of ethylene oxide and propylene oxide; polyacrylates and polymethacrylates including methacrylic acid/ethyl acrylate copolymers, methacrylic acid/methyl methacrylate copolymers, butyl methacrylate/2-dimethylaminoethyl methacrylate copolymers, poly(hydroxyalkyl acrylates), poly(hydroxyalkyl methacrylates); polyacrylamides; vinyl acetate polymers including copolymers of vinyl acetate and crotonic acid, partially hydrolyzed polyvinyl acetate; and oligo- and polysaccharides, including carrageenans, galactomannans and xanthan gum, Polyethylene glycol 2000, Polyethylene glycol 3000, Polyethylene glycol 6000, Polyethylene glycol 8000, Polyethylene glycol 10000, Polyethylene glycol 20000 and polyethylene glycol palmitostearate, Polyvinyl alcohol and acetate, Ethyl Vinayl acetate. and combinations thereof.

16. The BMPX composition of claim 1, wherein bonding agent is a polymer selected from the group consisting of: Poloxamer 188, Polyethylene glycol 2000, Polyethylene glycol 3000, Polyethylene glycol 6000, Polyethylene glycol 8000, Polyethylene glycol 10000, Polyethylene glycol 20000, and polyethylene glycol palmitostearate.

17. The BMPX composition of claim 1, wherein the bonding agent is one or more emulsifiers selected from polyethoxylated fatty acids including PEG-8 laurate, PEG-8 oleate, PEG-8 stearate, PEG-9 oleate, PEG-10 laurate, PEG-10 oleate, PEG-12 laurate, PEG-12 oleate, PEG-15 oleate, PEG-20 laurate and PEG-20 oleate; PEG-fatty acid diesters including PEG-20 dilaurate, PEG-20 dioleate, PEG-20 distearate, PEG-32 dilaurate and PEG-32 dioleate; PEG-fatty acid mono- and di-ester mixtures; polyethylene glycol glycerol fatty acid esters including PEG-20 glyceryl laurate, PEG-30 glyceryl laurate, PEG-40 glyceryl laurate, PEG-20 glyceryl oleate, and PEG-30 glyceryl oleate; alcohol-oil transesterification products including PEG-35 castor oil (Incrocas-35), PEG-40 hydrogenated castor oil (Cremophor® RH40), polyoxyl 35 castor oil (Cremophor EL), PEG-25 trioleate (TAGAT® TO), PEG-60 corn glycerides (Crovol M70), PEG-60 almond oil (Crovol A70), PEG-40 palm kernel oil (Crovol PK70), PEG-50 castor oil (Emalex C-50), PEG-50 hydrogenated castor oil (Emalex HC-50), PEG-8 caprylic/capric glycerides (Labrasol®), and PEG-6 caprylic/capric glycerides (Softigen® 767); transesterification products of oils and alcohols; polyglycerized fatty acids including polyglyceryl oleate (Plurol® Oleique), polyglyceryl-2 dioleate (Nikkol DGDO), and polyglyceryl-10 trioleate, polyglyceryl-10 laurate (Nikkol Decaglyn 1-L), polyglyceryl-10 oleate (Nikkol Decaglyn 1-0), and polyglyceryl-10 mono, dioleate (Caprol® PEG 860); propylene glycol fatty acid esters including propylene glycol monolaurate (Lauroglycol FCC), propylene glycol ricinoleate (Propymuls), propylene glycol monooleate (Myverol® P-06), propylene glycol dicaprylate/dicaprate (Captex® 200), and propylene glycol dioctanoate (Captex 800); mixtures of propylene glycol esters and glycerol esters including a mixture of oleic acid esters of propylene glycol and glycerol (Arlacel 186); mono- and diglycerides including glyceryl monooleate (Peceol), glyceryl ricinoleate, glyceryl laurate, glyceryl dilaurate (Capmul® GDL), glyceryl dioleate (Capmul GDO), glyceryl mono/dioleate (Capmul GMO-K), glyceryl caprylate/caprate (Capmul MCM), caprylic acid mono/diglycerides (Imwitor® 988), and mono- and diacetylated monoglycerides (Myvacet® 9-45); sterol and sterol derivatives including PEG-24 cholesterol ether (Solulan® C-24); polyethylene glycol sorbitan fatty acid esters including PEG-20 sorbitan monolaurate (Tween® 20), PEG-20 sorbitan monopalmitate (Tween 40), PEG-sorbitan monostearate (Tween 60), and PEG-20 sorbitan monooleate (polysorbate 80 or Tween 80); polyethylene glycol alkyl ethers including PEG-3 oleyl ether (Volpo 3) and PEG-4 lauryl ether (Brij 30); sugar esters including sucrose monopalmitate and sucrose monolaurate; polyethylene glycol alkyl phenols; polyoxyethylene-polyoxypropylene block copolymers including Synperonic® PE series (ICI); Pluronic® series (BASF), Emkalyx, Lutrol (BASF), Supronic, Monolan, Pluracare®, and Plurodac; sorbitan fatty acid esters including sorbitan monolaurate (Arlacel® 20), sorbitan monopalmitate (Span-40), sorbitan monooleate (Span-80), sorbitan monostearate, and sorbitan tristearate; lower alcohol fatty acid esters including hydrophobic surfactants include ethyl oleate (Crodamol EO), isopropyl myristate (Crodamol IPM), and isopropyl palmitate (Crodamol IPP), PEG-400 succinate, PEG 3350, tocopherol polyethyleneglycol (200-8000 MW) succinate, tocopherol polyethylene glycol 400 succinate, tocopherol polyethyleneglycol 1000 succinate (Vitamin E-TPGS, Eastman Chemical Co.), glycerol monolinoleate (Maisine®), propylene glycol monocaprylate (Capryol® 90); caprylocaproyl macrogol-8 glycerides (Labrosol®), glycerol dibehenate (Compritol® 888), glycerol distearate (Precirol®), lauroyl macrogol-32 glycerides (Gelucire® 44/14), and stearoyl macrogol-32 glycerides (Gelucire 50/13).

18. (canceled)

19. The BMPX composition of claim 1, wherein, the bonding agent is one or more fats selected from the group fats, triglycerides, hydrogenated natural oil or non-hydrogenated fat—fractionated Palm oils or others similar. In some embodiments, the hydrogenated natural oil is selected from the group consisting of hydrogenated canola oil, hydrogenated rapeseed oil, hydrogenated coconut oil, hydrogenated corn oil, hydrogenated cottonseed oil, hydrogenated olive oil, hydrogenated palm oil, hydrogenated peanut oil, hydrogenated safflower oil, hydrogenated sesame oil, hydrogenated soybean oil, hydrogenated sunflower oil, hydrogenated linseed oil, hydrogenated palm kernel oil, hydrogenated tung oil, hydrogenated jatropha oil, hydrogenated mustard oil, hydrogenated camelina oil, hydrogenated pennycress oil, hydrogenated castor oil, hydrogenated derivatives of these oils, and mixtures thereof.

20. (canceled)

21. The BMPX composition of claim 1, wherein the composition consists essentially of the solid particulate material and the solid bonding agent.

22. The BMPX composition of claim 1, wherein the composition is substantially free of organic solvents and organic solvent residuals.

23. (canceled)

24. The BMPX composition of claim 1, further comprising one or more excipients selected from the group consisting of: a binder, a disintegrant, a lubricant, a glidant, a surfactant, a stabilizer, a preservative, an alkalizing agent, an anti-adherent, a sweetener, a flavoring agent, a coloring agent, or a mixture thereof.

25. (canceled)

26. The composition of claim 1 comprising a dosage form selected from the group consisting essentially of: a tablet, a capsule, a lozenge, a caplet, a dry suspension, a rapid-melt tablet, a chew tablet, rapid-melt beads, a dry injectable formulation, an inhalable formulation, a transmucosal formulation, or a topical formulation.

27. A bonded microparticulate (BMPX) composition comprising a combination of a solid particulate material and a bonding agent, wherein the bonding agent holds particles of the solid particulate material together without regard to a property of the solid particulate material.

28. The BMPX composition of claim 27, wherein the property is density, hydrophilicity, hydrophobicity, crystalline form, amorphous form, or a combination thereof.

29-32. (canceled)

33. A method of making bonded microparticulates (BMPX) composition comprising:
softening a bonding agent at a temperature between room temperature and a melting point of the bonding agent;
contacting a solid particulate material with the softened bonding agent to form a mixture comprising the solid particulate material and the bonding agent; and
cooling the mixture to a predetermined temperature to produce bonded microparticulates.

34. The method of claim 33, further comprising mixing an amount of water with the softened bonding agent prior to contacting the solid particulate material with the softened bonding agent.

35. The method of claim 33, wherein the method excludes subsequent processing steps to arrive at the BMPX composition.

36. The method of claim 35, wherein the subsequent processing steps include drying or milling.

37-42. (canceled)
